(19) United States
(12) Patent Application Publication
(10) Pub. No.: US 2024/0165165 A1
(43) Pub. Date: May 23, 2024

(54) PHARMACEUTICAL COMPOSITIONS AND THEIR METHODS OF USE

(71) Applicant: The Trustees of Indiana University, Bloomington, IN (US)

(72) Inventor: Hiroki Yokota, Bloomington, IN (US)

(21) Appl. No.: 18/262,798

(22) PCT Filed: Jan. 25, 2022
(86) PCT No.: PCT/US2022/013661

§ 371 Date: Dec. 21, 2023

Related U.S. Application Data
(60) Provisional application No. 63/141,658, filed on Jan. 26, 2021.

(51) Int. Cl. A61K 35/32; C12N 5/077

(52) U.S. Cl. CPC A61K 35/32; C12N 5/0654; C12N 2500/90

(57) ABSTRACT

A pharmaceutical composition includes a cell-free conditioned medium (CM) or extract or concentrate thereof obtained from a mammalian cell culture medium comprising a cultured substantially homogenous non-cancerous mammalian cell population. At least a portion of the non-cancerous mammalian cell population is contacted by at least one small molecule cell growth signaling pathway activator before being cultured in the cell culture medium.

### CROSS-REFERENCE TO RELATED APPLICATIONS

This application claims priority to U.S. Provisional Patent Application No. 63/141,658, which was filed Jan. 26, 2021, the entire content of which is incorporated by reference herein.

### STATEMENT ON FEDERALLY SPONSORED RESEARCH OR DEVELOPMENT

This invention was made with government support under AR052144 and CA238555 awarded by National Institutes of Health. The Government has certain rights in the invention.

### FIELD

Embodiments disclosed herein are directed to conditioned media compositions and to their use to treat cancers.

### BACKGROUND

Cancer is characterized by proliferation of abnormal cells. Many cancer treatments include painful surgeries and chemotherapies with undesirable side effects. An ongoing, urgent need exists for new therapeutic interventions for cancer. The presently disclosed subject matter addresses this need.

### SUMMARY

In one aspect, the disclosure relates to pharmaceutical composition of a cell-free conditioned medium (CM) or extract or concentrate thereof obtained from a mammalian cell culture medium containing a cultured substantially homogenous non-cancerous mammalian cell population where a portion of the non-cancerous mammalian cell population is contacted by a small molecule cell growth signaling pathway activator before being cultured in the cell culture medium.

In another aspect, the disclosure relates to a kit comprising: a) a pharmaceutical composition according to the preceding aspect; b) a container; c) a label; and d) instructions that provide methods for administering the composition to a subject in need thereof.

In another aspect, the disclosure relates to a method to treat a cancer in a subject in need thereof by administering to the subject in need thereof a therapeutically effective amount of a cell-free conditioned medium (CM) or extract or concentrate thereof obtained from a mammalian cell culture medium containing a cultured substantially homogenous non-cancerous mammalian cell population where a portion of the non-cancerous mammalian cell population is contacted by a small molecule cell growth signaling pathway activator before being cultured in the cell culture medium.

In another aspect, the disclosure relates to a process to produce a conditioned medium (CM) by contacting non-cancerous mammalian cells by a small molecule cell growth signaling pathway activator to generate pre-treated non-cancerous mammalian cells; culturing the pre-treated non-cancerous mammalian cells in a mammalian cell culture medium for a period of time sufficient to condition the medium; removing the pre-treated non-cancerous mammalian cells from the culture medium; and, collecting the conditioned medium.

In another aspect, the disclosure relates to a method to identify an anti-tumor property in a conditioned medium (CM) by contacting non-cancerous mammalian cells by a small molecule cell growth signaling pathway activator to generate pre-treated non-cancerous mammalian cells; culturing the pre-treated non-cancerous mammalian cells in a mammalian cell culture medium to condition the medium; removing the pre-treated non-cancerous mammalian cells from the culture medium; collecting the conditioned medium; and, assaying the collected conditioned medium for an anti-tumor property.

This summary is not intended to identify key or essential features of the claimed subject matter, nor is it intended to be used as an aid in limiting the scope of the claimed subject matter. Furthermore, it is envisioned that alternative embodiments may combine features of two or more of the above-summarized embodiments. Further embodiments, forms, features, and aspects of the present application shall become apparent from the description and figures provided herewith.

### BRIEF DESCRIPTION OF THE DRAWINGS

The concepts described herein are illustrative by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 illustrates osteocyte-driven inhibition of tumorigenic behaviors of EO771 mammary tumor cells. Of note, CN=control (plain medium), CM=conditioned medium, A5=differentiated MLO-A5 osteocytes, and Y4=MLO-Y4 osteocytes, Scl=sclerostin, and β-cat=β-catenin. The single and double asterisks indicate p<0.05 and p<0.01, respectively. (a) Elevated levels of Sclerostin (Scl), Lrp5, and DMP1 in differentiated MLO-A5 osteocytes compared to MLO-Y4 osteocytes. (b&c) Lower levels of Scl and Lrp5 in A5 CM than Y4 CM. (d-f) Significant reduction in EdU-based proliferation, scratch-based migration, and TRANSWELL® invasion of EO771 mammary tumor cells by Y4 CM and A5 CM, compared to the control (plain medium). Scale bar: 200 (g) Reduction in Scl, Lrp5, β-catenin, MMP9, Runx2, TGFβ, Snail with an increase in cleaved caspase 3 in EO771 cells in response to Y4 CM and A5 CM.

FIG. 2 illustrates osteocyte-driven tumor-suppressing capabilities in vitro, ex vivo, and in vivo. CN=control (plain medium), and CM=conditioned medium. The single and double asterisks indicate p<0.05 and p<0.01, respectively (a) Reduction in scratch-based migration of 4T1.2 mammary tumor cells by A5 CM. Scale bar: 200 (b) Significant reduction in the cellular invasion of 2 sources of primary human breast cancer cells after 48 h treatment by A5 osteocyte-derived CM. Scale bar: 200 (c) Significant shrinkage of ex vivo breast cancer tissue fragments by A5 osteocyte-derived CM. On the red image at 0 h, the green image at 48 h is overlaid. Scale bar: 200 μm. (d) Significant reduction in the number of green-fluorescently-labeled EO771 mammary tumor cells (yellow arrows) in the lung in C57BL/6 mice by the co-injection with A5 osteocyte-derived CM in the assay. (e) Schematic diagram of a vinculin FRET biosensor: a tension sensitive sensor consists of the head and tail domains of vinculin with the elastic FRET module. In response to the strong (weak) force, the sensor exhibits a long (short) donor lifetime. (f) A5 CM-driven significant reduction in FRET lifetime of a vinculin biosensor in EO771 and MDA-MB-231 cells. The observed reduction in FRET lifetime indicates A5 CM's inhibitory role in molecular force and cellular migration. Scale bar: 20 μm.

FIG. 3 illustrates enhancement of tumor-suppressing capability by the overexpression of Lrp5 in A5 osteocytes. CN=control, CM=conditioned medium, pL5=Lrp5 plasmids, and shL5=Lrp5 shRNA. The single and double asterisks indicate p<0.05 and p<0.01, respectively. (a) Alteration in the Lrp5 level in A5 osteocytes by the overexpression and silencing of Lrp5. (b) Alterations in the levels of Sclerostin and Lrp5 in A5 CM in response to the overexpression and silencing of Lrp5 in A5 osteocytes. (c&d) Enhanced reduction in the EdU-based proliferation and TRANSWELL® invasion of EO771 cells in response to Lrp5-overexpressing A5 CM. Scale bar: 200 μm. (e) Significant shrinkage of ex vivo breast cancer tissue fragments by Lrp5-overexpressing A5 CM. On the red image at 0 h, the green image at 48 h is overlaid. Scale bar: 200 (f-h) Suppression of the anti-tumor action of A5 CM by silencing Lrp5 in A5 osteocytes in the EdU-based proliferation, TRANSWELL® invasion, and tumor spheroid assays using EO771 mammary tumor cells. Scale bar: 200 μm.

FIG. 4 illustrates the effects of the co-injection of A5 osteocytes to the tibia in C57BL/6 mice. pl=placebo, shL5=Lrp5 shRNA, pL5=Lrp5 plasmids, WT=wildtype, KO=Lrp5 conditional knockout, and CM=conditioned medium. The single and double asterisks indicate p<0.05 and p<0.01, respectively. (a) Representative X-ray and MR images of the tibia in the normal control (healthy group), placebo, A5 co-injected, and Lrp5 shRNA-treated A5 groups. The yellow arrows in MR images indicate a tumor-induced lesion in the tibia. MR image-based area ratio of the tumor-induced lesion is shown. Scale bar=1 mm. (b) Representative μCT images of the proximal tibia in the placebo, A5 co-injected, and Lrp5 shRNA-treated A5 groups. μCT-based parameters are shown, in which BV/TV=bone volume normalized by total volume, Tb.n=trabecular number, Tb.s=trabecular separation, and BMD=bone mineral density. Scale bar=500 μm. (c) Representative μCT images of the proximal tibia, showing the severe bone loss caused by tumor invasion in Lrp5 KO mice and its rescue by Lrp5-overexpressing osteocyte-derived CM. Scale bar=500 μm. (d) H&E stained proximal tibia of Lrp5 KO. Lrp5-overexpressing osteocyte-derived CM significantly reduced tumor growth. The green-shaded area showed the tumor-invaded region. Of note, tumor cells typically appeared hyperchromatic with an increased nucleoplasmic ratio. Scale bar=200 μm.

FIG. 5 illustrates regulation of tumor-promoting and tumor-suppressing genes by A5 osteoblasts. Of note, CN=control (plain medium), CM=conditioned medium, pL5=Lrp5 plasmids, pβ-cat=β-catenin plasmids, siβ=I3-catenin siRNA, and siT=Trail siRNA. (a) Comparison of 111 cytokine expression in A5 CM and Lrp5-overexpressing A5 CM. The levels of CXCL1 and CXCL5 were decreased in Lrp5-overexpressing CM. (b) Expression of CXCL1, CXCL5, WISP1, OPN, and M-CSF in A5 osteocytes with and without Lrp5 overexpression. (c) Elevation of CXCL1, CXCL5, WISP1, OPN, and M-CSF by TGFβ. (d) Proteins enriched in A5 osteocyte-derived CM. (e) Expression of TGFβ, TPM4, ANXA1, ANXA6, LIMA1, p53, and DSP in A5 osteocytes with and without Lrp5 overexpression. (f&g) Significant reduction in cellular proliferation and migration of EO771 cells after 48 h by β-catenin overexpressing osteocyte-derived CM. Scale bar: 200 μm. (h) Elevation of β-catenin and Trail by the overexpression of Lrp5 and β-catenin in A5 osteocytes and their conditioned medium. (i) Expression of Lrp5, MMP9, Runx2, TGFβ, Snail, cleaved caspase 3, and Trail in EO771 cells in response to β-catenin-overexpressing osteocyte-derived CM. (j) Suppression of the inhibitory effects of Lrp5-overexpressing osteocyte-derived CM by silencing of β-catenin. (k) Suppression of the inhibitory effects of β-catenin-overexpressing osteocyte-derived CM by silencing of Trail.

FIG. 6 illustrates effects of β-catenin-linked Wnt signaling in A5 osteocytes on tumor progression. Of note, CN=control (plain medium), CM=conditioned medium, pl=placebo, pL5=Lrp5 plasmids, and pβ-cat=β-catenin plasmids. The single and double asterisks indicate p<0.05 and p<0.01, respectively. (a-c) Significant reduction of mammary tumors and bone degradation in the tibia in C57BL/6 mice by the systemic administration of β-catenin-overexpressing osteocyte-derived CM. (d-e) Significant reduction in the number of TRAP-stained mature osteoclasts, as well as NFATc1 and Cathepsin K in RANKL-stimulated osteoclasts by Lrp5- and β-catenin-overexpressing osteocyte-derived CM. Scale bar: 200 μm. (f) Schematic illustration of the regulatory mechanism in tumor-osteocyte interactions. In this diagram, Lrp5 and β-catenin in osteocytes inhibit tumor progression by decreasing tumor-promoting genes (TGFβ, CXCL1, and CXCL5), and increasing tumor-suppressing genes (p53, ANXA6, and TPM4) and the apoptosis-inducing gene (Trail). In tumor cells, tumorigenic genes (Lrp5, Runx2, MMP9, and Snail) are downregulated. The overexpression of Lrp5 and β-catenin also inhibited osteoclastogenesis by downregulating NFATc1 and cathepsin K.

FIG. 7 illustrates effects of MLO-Y4 pre-osteocyte (Y4)-derived CM and fibroblast-derived CM on cellular behaviors of EO771 and 4T1.2 mammary tumor cells. Of note, CN=control (plain medium), CM=conditioned medium, pL5=Lrp5 plasmids, siL5=Lrp5 siRNA, and Fib=fibroblasts. The single and double asterisks indicate p<0.05 and p<0.01, respectively. Scale bar: 200 μm. (a) Significant reduction of cellular migration by Y4 CM and Lrp5-overexpressing Y4 CM in EO771 and 4T1.2 cells. (b) Effects of Y4 CM with and without Lrp5 siRNA treatment in EdU-based proliferation and migration of EO771 cells. (c&d) Promotion of the proliferation and migration by fibroblast-derived CM with and without Lrp5 overexpression in EO771 and 4T1.2 cells. Of note, fibroblasts were employed as a negative control in this study. (e) Growth promotion of ex vivo breast cancer tissue fragments by fibroblast-derived CM with and without Lrp5 overexpression.

FIG. 8 illustrates effects of primary human osteocyte (huO)-derived conditioned medium on MDA-MB-231 breast cancer cells, and PC-3 human prostate cancer cells. Of note, huO=human osteocytes, CN=control (plain medium), CM=conditioned medium, shL5=Lrp5 shRNA, and pL5=Lrp5 plasmids. The single and double asterisks indicate p<0.05 and p<0.01, respectively. Scale bar: 200 (a) Reduction in cellular proliferation, invasion, and migration of MDA-MB-231 cells by human osteocyte-derived CM with and without Lrp5 overexpression. (b&c) Inhibitory effect of human osteocyte-derived CM on EdU-based cellular proliferation and invasion in PC-3 prostate cancer cells.

FIG. 9 illustrates shrinkage of mammary tumor in C57BL/6 and NOD/Scid mice by the co-injection of A5 osteocytes, and the effects of Lrp6 silencing in EO771 tumor cells and A5 osteocytes on mammary tumors in C57BL/6 mice. Of note, pL5=Lrp5 plasmids, shL5=Lrp5 shRNA, shCN=control shRNA, shL5=Lrp5 shRNA, and slL6=Lrp6 shRNA. The single and double asterisks indicate p<0.05 and p<0.01, respectively. Scale bar: 1 cm. (a) Schematic illustration of C57BL/6 female, showing the inoculation of EO771 mammary tumor cells with and without the co-injection of A5 osteocytes to the mammary fat pad. The results showed a significant reduction in tumor progression by the co-injection of A5 osteocytes (N=10). (b-d) Effects of osteocyte inoculation with and without Lrp5 shRNA or Lrp5 plasmids on the tumor size and weight in NOD/Scid mice (N=10). The pink ribbon indicates no detectable tumor. The histological section shows GFP-labeled osteocytes in the mammary tumor. (e&f) No significant effect of Lrp6 silencing in EO771 cells (N=10). (g&h) Differential effects of the silencing of Lrp5 and Lrp6 in A5 osteocytes. Lrp5 silencing suppressed the anti-tumor capability with A5 osteocytes, but Lrp6 silencing did not significantly alter it (N=10).

FIG. 10 illustrates protection of tumor-induced osteolysis in the tibia in C57BL/6 mice by the co-injection of A5 osteocytes, and by Lrp5 silencing in EO771 tumor cells. Of note, shCN=control shRNA, shL5=Lrp5 shRNA, CN=normal control, WT=wildtype, and KO=knockout. The single and double asterisks indicate p<0.05 and p<0.01, respectively. (a) H&E-stained images of the proximal tibia for the placebo, A5 osteocyte-injected group, and Lrp5 shRNA-treated osteocyte group. The relative tumor area in the proximal tibia is presented (N=8). Scale bar: 100 μm. (b) Effects by silencing of Lrp5 in EO771 on the tumor size and weight (N=8). Scale bar: 500 mm. (c) Reduction in tibial osteolysis by silencing of Lrp5 in EO771 tumor cells (N=8). Of note, BV/TV=bone volume normalized by total volume, BMD=bone mineral density, Tb.n=trabecular number, and Tb.s=trabecular separation. Scale bar: 500 (d) Reduction in BV/TV in the tibia caused by Lrp5 deletion in the osteocytes (N=8). Scale bar: 500 (e) Tumor-induced decrease of BV/TV in the tibia was more severe than the decrease caused by Lrp5 deletion.

FIG. 11 illustrates effects of Lrp5 plasmids and BML284, an activator of Wnt signaling on tumor progression. Of note, CN=control (plain medium), CM=conditioned medium, pL5=Lrp5 plasmids, siβ or siβ-c=β-catenin siRNA, and BML=BML284. The double asterisk indicates p<0.01. (a) Relative intensity levels of tumor-promoting cytokines/chemokines in A5 osteocyte-derived CM with and without Lrp5 overexpression. (b) Elevation of apoptosis-linked genes (CYCS, HIF1α, and APT1) in EO771 cells of β-catenin-overexpressing osteocyte-derived CM. (c) Suppression of the inhibitory effect of Lrp5-overexpressing A5 CM by RNA interference with β-catenin siRNA on EdU-based cellular proliferation and invasion in EO771 breast cancer cells. Scale bar: 200 (d) Levels of Sclerostin and Lrp5 in A5 CM with and without β-catenin-overexpression and BML284 treatment. (e) Shrinkage of ex vivo breast cancer tissue fragments by BML284-treated, osteocyte-derived CM. Scale bar: 200 (f) Downregulation of Lrp5, MMP9, Runx2, Snail, and TGFβ in EO771 tumor cells in response to BML284-treated, osteocyte-derived CM. (g) Reduction in the number of green-fluorescently-labeled EO771 mammary tumor cells in the lung by BML284 co-injection in the extravasation assay. (h) Reduction in mammary tumors in CL57BL/6 mice by systemic administration of BML284-treated, osteocyte-derived CM. Scale bar: 1 cm.

FIG. 12 illustrates gene regulation in β-catenin-overexpressing A5 osteocytes and EO771 mammary tumor cells, and effects of β-catenin-overexpressing in A5 osteocytes on tibia tumor progression. Of note, NC=non-specific control vector, pβ-cat=β-catenin plasmids, CN=control (plain medium), CM=conditioned medium, and siT=Trail siRNA. (a) Expression of tumor-suppressing genes (TPM4, ANXA1, ANXA6, LIMA1, p53, and DSP) in β-catenin overexpressing osteocytes. (b) Expression of CXCL1, CXCL5, WISP1, OPN, and M-CSF in EO771 cells in response to β-catenin overexpressing osteocyte-derived CM. (c) Expression of tumor-promoting genes (Lrp5, MMP9, Runx2, and Snail) in EO771 cells in response to TGFβ and CXCL5. (d) Expression of Lrp5, MMP9, Runx2, TGFβ, Snail, and cleaved caspase 3 in EO771 cells in response to TPM4, ANXA6, and Trial. (e) Trail expression in A5 osteocytes in response to β-catenin plasmids with and without Trail siRNA. (f) Inhibitory effect of A5 CM with and without β-catenin-overexpression on cellular proliferation and invasion in EO771 breast cancer cells. Of note, RNA interference with Trail siRNA partially suppressed the inhibitory effect with the overexpression of β-catenin. Scale bar: 200 μm. (g) Reduction of bone degradation in the tibia in C57BL/6 mice by the systemic administration of β-catenin-overexpressing osteocyte-derived CM. Scale bar: 1 mm.

FIG. 13 illustrates involvement of Runx2 in the anti-tumor capability of A5 osteocytes. Of note, CN=control (plain medium), CM=conditioned medium, shCN=control shRNA, and shR2=Runx2 shRNA. The single and double asterisks indicate p<0.05 and p<0.01, respectively. (a) Reduction in Runx2 and MMP9 in 2 sources of primary human breast cancer cells by A5 osteocyte CM. (b) Reduction in FRET lifetime (weakening molecular force) in EO771 tumor cells by Runx2 silencing in A5 osteocytes. Scale bar: 20 μm. (c) Shrinkage of the mammary tumor in C57BL/6 mice by silencing Runx2 in EO771 tumor cells. Scale bar: 1 cm.

FIG. 14 illustrates effects of the culture condition and vinculin on the tumor-suppressing capability of mesenchymal stem cells (“MSCs”). CN=control, adh=adherent culture, susp=suspension culture, and siVinc=vinculin siRNA. The single and double asterisks indicate p<0.05 and 0.01, respectively. (A) Reduction in MTT-based viability of 4T1.2 mammary tumor cells by MSC CM in suspension culture. (B) Decrease in the TRANSWELL®-based invasion of 4T1.2 cells by MSC CM in suspension culture. (C&D) Inhibition of MTT-based viability and TRANSWELL®-based invasion of MDA-MB-231 breast cancer cells by MSC CM in suspension culture. (E&F) Reduction in the level of vinculin in suspension culture and by RNA interference with vinculin siRNA. (G&H) Reduction in MTT-based viability and scratch-based migration of 4T1.2 cells by vinculin-silenced MSC CM.

FIG. 15 illustrates tumor-suppressing effects of Lrp5-overexpressing MSC CM in EO771 mammary tumor cells. CN=control, Lrp5=Lrp5 plasmids, and siL5=Lrp5 siRNA. The double asterisk indicates p<0.01. (A-C) Overexpression of Lrp5 in MSCs and the inhibition of scratch-based cellular migration, EdU-based proliferation, and TRANSWELL® invasion by Lrp5-overexpressing MSC CM. (D&E) Silencing of Lrp5 in MSCs and the increase in EdU-based proliferation and TRANSWELL® invasion by Lrp5-silenced MSC CM. (F) Comparison of 111 cytokines and chemokines in MSC CMs with and without Lrp5 overexpression. Compared to MSC CM, Lrp5-overexpressing MSC CM elevated the levels of six proteins (CXCL2-CXC motif chemokine ligand 2; GM-CSF—granulocyte-macrophage colony-stimulating factor; IL6-interleukin 6; LIF—leukemia inhibitory factor; DLK1-delta like non-canonical Notch ligand 1; and MRP—multidrug resistance protein) and reduced the level of one protein (IL27-interleukin 27).

FIG. 16 illustrates tumor-suppressing effects of β-catenin-overexpressing MSC CM in EO771 mammary tumor cells, C57BL/6 mice, and BALB/c mice. CN=control, Lrp5=Lrp5 plasmids, siL5=Lrp5 siRNA, β-cat=β-catenin plasmids, siβ=β-catenin siRNA, and pl=placebo. The single and double asterisks indicate p<0.05 and 0.01, respectively. (A-C) Reduction in EdU-based proliferation and TRANSWELL®-based invasion by β-catenin-overexpressing MSC CM, and their increase by β-catenin-silenced MSC CM. (D) Downregulation of Lrp5, Runx2, MMP9, and Snail in EO771 cells by Lrp5- and β-catenin-overexpressing MSC CM, and their upregulation by Lrp5- and β-catenin-silenced MSC CM. (E) Downregulation of CXCL2 and LIF, and upregulation of p53 and Trail in Lrp5- and β-catenin-overexpressing MSC CM. The responses are reversed by Lrp5- and β-catenin-silenced MSC CM. (F) Reduction in the weight of mammary tumors by the co-injection of Lrp5-overexpressing MSCs in C57BL/6 mice. (G) Reduction in the weight of mammary tumors by the daily intravenous administration of β-catenin-overexpressing MSC CM in BALB/c mice. (H) Protection of the tumor-invaded proximal tibia by the daily intravenous administration of β-catenin-overexpressing MSC CM. BV/TV=bone volume ratio, BMD=bone mineral density, Tb.N=trabecular number, and Tb. Sp=trabecular separation.

FIG. 17 illustrates tumor-suppressing effects of Snail-overexpressing MSC CM in EO771 cells and C57BL/6 mice. CN=control, Snail=Snail plasmids, and siSnail=Snail siRNA. The single and double asterisks indicate p<0.05 and 0.01, respectively. (A-C) Overexpression of Snail, and inhibition of EdU-based proliferation and TRANSWELL®-based invasion by Snail-overexpressing MSC CM. (D-F) Silencing of Snail in MSCs and the increase in EdU-based proliferation and TRANSWELL® invasion by Snail-silenced MSC CM. (G) Expression of tumorigenic genes (Lrp5, MMP9, Runx2, TGFβ, and Snail) in EO771 cells in response to Snail-overexpressing and Snail-silenced MSC CMs. (H) Reduction in the weight of mammary tumors by the daily intravenous administration of Snail-overexpressing MSC CM. (I) Reduction in bone loss in the proximal tibia by the intravenous administration of Snail-overexpressing MSC CM. Of note, BV/TV=bone volume ratio, BMD=bone mineral density, Tb.N=trabecular number, and Tb. Sp=trabecular separation. (J) Scattered distribution of EO771 tumor cells (highlighted in green) in the proximal tibia and the reduced size of tumor clusters by Snail-overexpressing MSC CM. (K) Shrinkage of ex vivo breast cancer tissue fragments by Snail-overexpressing MSC CM.

FIG. 18 illustrates induction of tumor-suppressing capability by the overexpression of Akt in MSCs. CN=control, Akt pl=Akt plasmids, and siAkt=Akt siRNA. The single and double asterisks indicate p<0.05 and 0.01, respectively. (A&B) Significant reduction in EdU-based proliferation of 4T1.2 cells by Akt-overexpressing MSC CM. (C&D) Increase in EdU-based proliferation of 4T1.2 cells by Akt-silenced MSC CM. (E&F) Significant decrease in TRANSWELL®-based invasion of 4T1.2 cells by Akt-overexpressing MSC CM, and the opposite response by Akt-silenced MSC CM. (G) Shrinkage of ex vivo breast cancer tissues by Akt-overexpressing and YS49-treated MSC CMs. (H) Cell competition assay for 4T1.2 tumor spheroids in response to MSC spheroids and MSC CMs, Reduction in three-dimensional 4T1.2 tumor spheroids in response to Akt-overexpressing MSCs.

FIG. 19 illustrates suppression of tumor growth in the mammary fat pad and proximal tibia in BALB/c mice by the administration of MSC CM in suspension culture, Akt-overexpressing, and Y549-treated MSC CMs. Of note, pl=placebo (inoculation of 4T1.2 cells without treatment), susp=suspension culture. The single and double asterisks indicate p<0.05 and 0.01, respectively. (A&B) Reduction in the weight of mammary tumors by the daily intravenous administration of MSC CM in suspension culture, and Akt-overexpressing and Y549-treated MSC CMs. (B&C) Protection of the tumor-invaded proximal tibia by the daily intravenous administration of MSC CMs. BV/TV=bone volume ratio, BMD=bone mineral density, Tb.N=trabecular number, and Tb. Sp=trabecular separation. (D) Significant reduction in 4T1.2 tumor cells (highlighted in green) in the proximal tibia and the average tumor cluster size by the daily intravenous administration of MSC CM in suspension culture, and Akt-overexpressing and Y549-treated MSC CMs.

FIG. 20 illustrates mass spectrometry-based proteomics analysis and the tumor-suppressing effect of Hsp90ab1. Of note, siH or siH90ab1=Hsp90ab1 siRNA, CN=control, and CM=conditioned medium. The single and double asterisks indicate p<0.05 and 0.01, respectively. (A) List of the selected proteins, enriched in Akt-overexpressing and Y549-treated MSC CM. (B) Significant reduction in MTT-based viability of 4T1.2 cells by the incubation with 23 recombinant proteins, including Hsp90ab1, Calr, and Ppib. (C) Upregulation of Hsp90ab1, Calr, and Ppib in MSC CMs by the overexpression of Akt, Lrp5, β-catenin, and Snail, as well as the administration of YS49 (D) Significant reduction in scratch-based migration of 4T1.2 cells by Hsp90ab1. (E) Downregulation of Lrp5, Runx2, MMP9, and Snail in 4T1.2 cells by Hsp90ab1. (F&G) Silencing of Hsp90ab1 in MSCs and the increase in MTT-based proliferation and scratch-based migration by Hsp90ab1-silenced MSC CM. (H) Increase in the levels of tumorigenic genes (Lrp5, MMP9, Runx2, TGFβ, and Snail) in 4T1.2 cells in response to Hsp90ab1-silenced MSC CM. (I) Upregulation of CXCL2 and LIF, and downregulation of p53 and Trail in Hsp90ab1-silenced MSC CMs.

FIG. 21 illustrates effects of Calr and Ppib on 4T1.2 mammary tumor cells. The double asterisk indicates p<0.01. CN=control, and CM=conditioned medium. (A&B) Reduction in TRANSWELL® invasion and EdU-based proliferation by recombinant Calr and Ppib proteins. (C) Reduction in Lrp5, MMP9, Runx2, and Snail by recombinant Calr and Ppib proteins. (D) Overexpression of Calr and Ppib in MSCs. (E&F) Reduction in TRANSWELL® invasion and EdU-based proliferation of 4T1.2 cells in response to Calr- and Ppib-overexpressing MSC CM. (G) Downregulation of Lrp5, MMP9, Runx2, and Snail in 4T1.2 cells in response to Calr- and Ppib-overexpressing MSC CM. (H) Upregulation of p53 in Calr- and Ppib-overexpressing MSC CM. (I) Elevation of phosphorylated eIF2a in 4T1.2 cells in response to Calr and Ppib recombinant proteins.

FIG. 22 illustrates MTT-based tumor selectivity of the inhibition of the growth of tumor cells by Lrp5 CM, Akt C M, Calr, and Ppib. The tumor selectivity was defined as the ratio of (reduction in MTT-based viability of tumor cells) to (reduction in MTT-based viability of non-tumor cells). The double asterisk indicates p<0.01. (A) Comparison of MTT-based viability of tumor cells (4T1.2, EO771, and MDA-MB-231 cells) and non-tumor cells (MSCs, and MLO-A5 osteocytes). (B&C) Summary of the tumor selectivity.

FIG. 23 illustrates inhibition of the development of osteoclasts and the regulatory mechanism with MSC CM. The double asterisk indicates p<0.01. (A&B) Significant reduction in TRAP-positive multi-nucleated osteoclasts (>3 nuclei) by MSC CMs with the overexpression of Akt, Lrp5, β-catenin, and Snail. RAW264.7 pre-osteoclasts were stimulated their differentiation by RANKL. (C) Significant downregulation of NFATc1 and cathepsin K in RANKL-stimulated pre-osteoclasts by MSC CMs with the overexpression of Akt, Lrp5, β-catenin, and Snail. (D) Regulatory mechanism by MSC CM for the suppression of tumor progression and osteoclast development.

FIG. 24 illustrates effects of MSC CM in suspension culture and RNA interference with vinculin siRNA on the MTT-based viability of EO771 mammary tumor cells. The double asterisk indicates p<0.01.

FIG. 25 illustrates effects of Lrp5-overexpressing MSC CM on the behaviors of TRAMP prostate tumor cells. The double asterisk indicates p<0.01. (A&B) Reduction in EdU-based proliferation and TRANSWELL®-based invasion of TRAMP cells in response to Lrp5-overexpressing MSC CM. (C) Reduced expression levels of Lrp5, Runx2, MMP9, and Snail in TRAMP cells in response to Lrp5-overexpressing MSC CM.

FIG. 26 illustrates effect of Snail-overexpressing MSC CM on EO771 mammary tumor cells and TRAMP prostate tumor cells. Snail-MSC CM=Snail-overexpressing MSC CM, and siSnail=Snail siRNA. The double asterisk indicates p<0.01. (A&B) Reduction in scratch-based migration and three-dimensional EO771 tumor spheroid size in response to Snail-overexpressing MSC CM. (C) Downregulation of CXCL2 and LIF, and upregulation of p53 and Trail by Snail-overexpressing MSC CM. The responses were reversed by Snail-silenced MSC CM. (D) Reduction in the TRANSWELL®-based invasion of TRAMP cells in response to Snail-overexpressing MSC CM. (E) Reduced expression levels of Lrp5, Runx2, MMP9, and Snail in TRAMP prostate tumor cells in response to Snail-overexpressing MSC CM.

FIG. 27 illustrates effects of Akt-overexpressing/silenced MSC CMs and YS49 on 4T1.2, EO771, and MDA-MB-231 cancer cells. Of note, susp=suspension culture, Akt pl=Akt plasmids, siAkt=Akt siRNA. The single and double asterisks indicate p<0.05 and 0.01, respectively. (A-C) Elevation of phosphorylated Akt by YS49, and a significant reduction in EdU-based proliferation and TRANSWELL®-based invasion of 4T1.2 cells by Y549-treated MSC CM. (D) Altered expression levels of Lrp5, MMP9, Runx2, TGFβ, and Snail in response to Akt-overexpressing/silenced or Y549-treated MSC CMs. (E) Reduction in MTT-based viability of EO771 mammary tumor cells by MSC CM in suspension culture and Akt-overexpressing MSC CM. (F) Reduced expression levels of Lrp5, MMP9, Runx2, Snail, and TGFβ in EO771 cells in response to Akt-overexpressing MSC CM. (G&H) Reduction in MTT-based viability and TRANSWELL®-based invasion of MDA-MB-231 breast cancer cells by MSC CM in suspension culture and Akt-overexpressing MSC CM.

FIG. 28 illustrates cell competition assay for 4T1.2 tumor spheroids in response to MSC spheroids and MSC CMs, in which Lrp5, β-catenin or Snail was overexpressed. (A) Reduction in three-dimensional 4T1.2 tumor spheroids in response to Lrp5-, I3-cat-, and Snail-overexpressing MSCs. (B) Reduction in three-dimensional 4T1.2 tumor spheroids in response to Lrp5-, β-catenin, and Snail-overexpressing MSC CMs.

FIG. 29 illustrates effects of Akt, YS49, Hsp90ab1, Calr, Ppib, and Hsp90aa1. CN=control, and C=conditioned medium. (A) Downregulation of CXCL2 and LIF and upregulation of p53 and Trail in Akt-overexpressing and Y549-treated MSC CMs. (B) Reduction of Hsp90ab1, Calr, and Ppib in MSC CM by the silencing of Akt, Lrp5, β-catenin, and Snail. (C) Undetectable change of Lrp5, Runx2, MMP9, and Snail in 4T1.2 cells in response to H90aa1 recombinant proteins. (D) Reduction in the MTT-based cell viability of 4T1.2 cells in response to HSF1 recombinant proteins.

FIG. 30 illustrates Hsp90ab1 as an extracellular tumor suppressor in 4T1.2 mammary tumor cells. The single and double asterisks indicate p<0.05 and 0.01, respectively. CN=control, CM=conditioned medium, Akt=Akt overexpression, 17-AGG=Hsp90 inhibitor (Tanespimycin), and heat=heat shock at 42° C. for 1 h. (A) Regulation of Hsp90ab1, CXCL2, LIF, p53, and Trail by the inhibition of Hsp90. (B&C) Suppression of MSC CM-driven reduction in the EdU-based proliferation and scratch-based migration by the inhibition of Hsp90. (D) Suppression of MSC CM-driven reduction in Lrp5, MMP9, Runx2, and Snail by the inhibition of Hsp90. (E-H) Enhancement of the tumor-suppressing action of MSC CM by heat shock at 42° C. for 1 h.

FIG. 31 illustrates suppression of the cellular proliferation, invasion, and migration in EO771 tumor cells by osteocyte-derived CM with and without Lrp5 overexpression. Of note, CN=control and CM=conditioned medium. The double asterisk indicates p<0.01. The error bar indicates a standard deviation. (A) Representative images of MLO-A5 osteocytes (hematoxylin stained). (B) Transfection of Lrp5 plasmids in A5 osteocytes. (C-E) Reduction in MTT-based cellular viability, scratch-based migration, and TRANSWELL® invasion by osteocyte-derived CM. (F) Shrinkage of tumor spheroids in response to osteocyte-derived CM.

FIG. 32 illustrates suppression of the growth of mammary tumors and tumor-driven osteolysis by osteocytes and their conditioned medium. The double asterisk indicates p<0.01. The error bar indicates a standard deviation. pl=placebo, and CM=conditioned medium. (A) Inhibition of the growth of mammary tumors by the co-injection of osteocytes and Lrp5-overexpressing osteocytes. (B) Reduction in the size of mammary tumors by the administration of b-catenin-overexpressing osteocyte-derived CM. (C) Prevention of bone loss in the proximal tibia by the co-injection of osteocytes and Lrp5-overexpressing osteocytes. The red arrowheads indicate the destructed area by tumor cells. (D) Protection of bone by the administration of b-catenin-overexpressing osteocyte-derived CM. The red arrowhead indicates the destructed area by tumor cells.

FIG. 33 illustrates suppression of the growth of brain tumors by the co-injection of Lrp5-overexpressed osteocytes in C57BL/6 mice (N=8 per group). Of note, CN=control. The single and double asterisks indicate p<0.05 and p<0.01, respectively. The error bar indicates a standard deviation. (A&B) Activity score and body weight for 2 weeks. The red arrowheads indicate osteocyte injections. (C) Representative images of the whole brain with the brain damage score. (D) Histological images and the comparison of tumor areas in the coronal section.

FIG. 34 illustrates anti-tumor effects of IL1ra overexpression in osteocytes. Of note, CN=control, and CM=conditioned medium. The single and double asterisks indicate p<0.05 and p<0.01, respectively. The error bar indicates a standard deviation. (A) Overexpression of IL1ra in osteocytes by the transfection of IL1ra plasmids. (B-D) Reduction in MTT-based cellular viability, scratch-based migration, and TRANSWELL® invasion in EO771 cells by IL1ra-overexpressing CM. (E) Reduction in the size and weight of mammary tumors in C57BL/6 mice (N=8-12 per group) by the co-injection of IL1ra-overexpressing osteocytes. (F) Suppressive effects of osteocytes, overexpressed with IL-1ra, and/or Lrp5 in C57BL/6 mice. Whole brains in the three groups (placebo, injection of IL1ra-overexpressing osteocytes, and injection of IL1ra/Lrp5-overexpressing osteocytes; N=8 per group). (G) Histological analysis, showing that the osteocyte injection significantly suppressed tumor growth, and the overexpression of IL1ra and Lrp5 further reduced tumor progression.

FIG. 35 illustrates anti-tumor effect of osteocyte-derived CM with β-catenin overexpression on brain tumors. Of note, β-cat=β-catenin, CN=control, and CM=conditioned medium. The double asterisks indicate p<0.01. The error bar indicates a standard deviation. (A-C) Reduction in MTT-based cell viability, scratch-based migration, and TRANSWELL® invasion in EO771 cells by β-catenin overexpressing CM. (D&E) Activity score and body weight for 2 weeks (N=8 per group). (F) Whole brains in the two groups (placebo and the injection of β-catenin overexpressing CM) with the brain damage score. (G) Histological analysis, showing that osteocyte-derived CM with the overexpression of β-catenin significantly suppressed tumor growth.

FIG. 36 illustrates transport analysis of conditioned medium through the skull and the alterations in gene expression. Of note, FP=fluphenazine, β-cat=β-catenin, CN=control, and CM=conditioned medium. The single and double asterisks indicate p<0.05 and p<0.01, respectively. The error bar indicates a standard deviation. (A) Coronal sections of the skull. The red mark on the skull image indicates the injection site of EO771 tumor cells. (B) Temporal changes in the diffusion-linked ratio. (C) Transport analysis with MTT-based viability, in which EO771 cells were cultured on the inside of the skull. FP and β-catenin-overexpressing CM in the outside of the skull decreased the viability of tumor cells on the inside of the skull. Of note, 10 μM of FP (an antagonist of dopamine D2 receptor) in the inside of the skull was employed as a positive control for tumor suppression. (D) Changes in the size of human mammary tumor fragments. In the control groups, the transport of FP and CM was blocked by the coverage of the skull with Aluminum foil. The administration of FP and CM in the outside of the skull shrunk the mammary tumor fragments in the inside of the skull. Of note, 10 μM of FP in the inside of the skull was employed as a positive control for tumor suppression. (E) Reduction in IL1β, Runx2, MMP9, TGFβ, and Snail in EO771 cells by osteocyte-derived CM with and without the overexpression of Lrp5, IL1ra, and β-catenin. (F) Elevation in p53, TRAIL, LIMA1, and DSP, as well as the reduction in CXCL1 and CXCL5 in osteocyte-derived CM by the overexpression of β-catenin.

FIG. 37 illustrates anti-tumor actions of osteocyte-derived conditioned medium and the tumor-suppressing role of histone H4 in EO771 mammary tumor cells. CN=control, CM=conditioned medium, H4=histone H4, and siH4=histone H4 siRNA. The single and double asterisks indicate p<0.05 and 0.01, respectively. The error bar indicates a standard deviation. (A) Eight tumor-suppressing protein candidates, predicted by whole-genome proteomics. (B) Effects of the predicted candidates on the MTT-based viability of EO771 mammary tumor cells. (C) Elevation of histone H4 in osteocyte-derived CM by the overexpression of Lrp5, IL1ra, and (3-catenin. (D) Reduction in MTT-based viability by histone H4 and its suppression by histone H4 siRNA. (E&F) Reduction in the scratch-based migration and EdU-based proliferation by histone H4. (G&H) Suppression of the reduced migration and proliferation by histone H4 siRNA. (I) Reduction in IL1β, Runx2, MMP9, TGFβ, and Snail by histone H4, and the suspension of their reduction by histone H4 siRNA. (J) Reduction in p53, TRAIL, LIMA1, and DSP, as well as the elevation of CXCL1 and CXCL5 in osteocyte-derived CM by histone H4 siRNA.

FIG. 38 illustrates characterization of CMs derived from astrocytes and mesenchymal stem cells, and the regulatory mechanism of osteocyte-driven tumor suppression. The double asterisk indicates p<0.01. The error bar indicates a standard deviation. CN=control, and CM=conditioned medium. (A-C) Scratch-based migration and MTT-based viability of EO771 cells in response to astrocyte-derived CM with and without Lrp5 overexpression. (D) Elevation in MMP9, Runx2, and Snail in EO771 cells by astrocyte-derived CM. (E) MTT-based viability of EO771 cells in response to MSC-derived CM with and without Lrp5 overexpression. (F) Reduction in MMP9, Runx2, and Snail in EO771 cells by MSC-derived CM. (G) Schematic illustration of the regulatory mechanism of the anti-tumor action of osteocytes. Osteocytes have the potential to suppress the expression of IL1β, MMP9, Runx2, TGFβ, and Snail in tumor cells. The overexpression of Lrp5, β-catenin, and IL1ra strengthens their anti-tumor capability. In particular, the overexpression of β-catenin elevated extracellular histone H4 that served as a tumor suppressor. The elevated histone H4 downregulated the tumor promotors (CXCL1 and CXCL5) and upregulated the tumor suppressors and apoptosis inducers (LIMA1, DSP, TRAIL, c-cas3, and p53).

FIG. 39 illustrates anti-tumor effects of IL1ra overexpression in osteocytes. Of note, CN=control, and CM=conditioned medium. The double asterisks indicate p<0.01. (A-C) Reduction in the size of tumor spheroids, migration in the scratch assay, and TRANSWELL® invasion in response to osteocyte-derived CM with and without the overexpression of IL1ra.

FIG. 40 illustrates suppressive effect of osteocytes, overexpressed with IL1ra, and/or Lrp5 in C57BL/6 mice (N=8 per group). (A&B) Activity score and body weight for 2 weeks. The red arrowheads indicate the days osteocytes were injected.

FIG. 41 illustrates cellular behavior of EO771 mammary tumor cells in response to β-catenin-overexpressing osteocyte-derived condition medium. Of note, CN=control, CM=conditioned medium, and β-cat=β-catenin. (A) Scratch-based migration assay. (B) TRANSWELL® invasion assay.

FIG. 42 illustrates effects of β-catenin-overexpressing osteocyte-derived condition medium and histone H4 on MTT-based viability of EO771 mammary tumor cells, MLO-A5 osteocytes, MC3T3 osteoblasts, and MSCs. (A) MTT viability of EO771 cells. (B) MTT viability of non-tumor cells such as MLO-A5 osteocytes, MC3T3 osteoblasts, and MSCs. (C) Tumor selectivity of β-catenin-overexpressing osteocyte-derived condition medium and histone H4. The tumor selectivity is defined as (MTT reduction in tumor cells) divided by (MTT reduction in non-tumor cells). The tumor selectivity is above 1 in all cases, indicating that the inhibitory effects of these agents are selective to tumor cells.

FIG. 43 illustrates inhibitory effects of Lrp5-Oy CM on TRAMP-C2ras prostate tumor cells. CN=control, CM=conditioned medium, Oy CM=osteocyte-derived CM, and Lrp5=Lrp5 overexpression. The data are presented as mean±S.D. (n=3). The double asterisk indicates p<0.01. (A-C) Reduction in MTT-based viability and EdU-based proliferation by Lrp5-Oy CM. (D) Inhibition of TRANSWELL® invasion by Lrp5-Oy CM. (E) Reduction in the growth of 3D tumor spheroids by Lrp5-Oy CM.

FIG. 44 illustrates inhibitory effects of Lrp5-Oy CM on PC-3 prostate cancer cells, and ex vivo human prostate cancer tissues. CN=control, CM=conditioned medium, Oy CM=osteocyte-derived CM, Lrp5=Lrp5 overexpression, and pl=placebo. The data are presented as mean±S.D. (n=3). The single, double, and triple asterisks indicate p<0.05, 0.01 and 0.001, respectively. (A&B) Inhibition of MTT-based viability, EdU-based proliferation of PC-3 prostate cancer cells by Lrp5-Oy CM. (C) Reduction in TRANSWELL® invasion of PC-3 prostate cancer cells by Lrp5-Oy CM. (D) Reduction in the growth of PC-3 prostate cancer spheroids by Lrp5-Oy CM. (E) Inhibition of the growth of prostate cancer tissue fragments by Lrp5-Oy CM.

FIG. 45 illustrates inhibitory effects of Lrp5-Oy CM on tumor migration and invasion. CN=control, Oy CM=osteocyte-derived CM, and Lrp5=Lrp5 overexpression. The double and triple asterisks indicate p<0.01 and 0.001, respectively. (A&B) Inhibition of scratch-based migration by Lrp5-Oy CM in TRAMP cells and PC-3 cells. (C) Increase in FRET efficiency in the vinculin biosensor by Lrp5-Oy CM. The observed increase in FRET efficiency implies a decrease in vinculin-mediated molecular force at focal adhesions. (D) Tumor-invaded lungs in the extravasation assay. The yellow arrows in the placebo group indicate fluorescently labeled TRAMP tumor cells. The co-injection of Lrp5-Oy CM reduced tumor invasion to the lungs.

FIG. 46 illustrates protection of the TRAMP tumor cell-invaded proximal tibia by the co-injection of Lrp5-overexpressing osteocytes. CN=control (no tumor injection), pl=placebo, Oy=osteocytes, and Lrp5-Oy=Lrp5-overexpressing Oy. The single and double asterisks indicate p<0.05 and 0.01, respectively. (A&B) TRAMP prostate cancer cell-invaded tibia and fibula of C57BL/6 male mice. (C&D) EO771 mammary tumor cell-invaded tibia and fibula of C57BL/6 female mice. (E) Protection of trabecular bone in the proximal tibia by the co-injection of Oy and Lrp5-Oy. BMD=bone mineral density, BV/TV=bone volume ratio, Tb.N=trabecular number, and Tb. Sp=trabecular separation. (F) Reduction in the tumor-invaded area in the proximal tibia by the co-injection of Oy and Lrp5-Oy. The green dotted area indicates the tumor-invaded region.

FIG. 47 illustrates severe tumor-induced bone loss in Lrp5 conditional knockout mice and the rescue by the injection of Lrp5-overexpressing osteocytes. WT=wildtype, KO=conditional knockout of Lrp5 in osteocytes, Oy=osteocytes, and Lrp5 CM=Lrp5-overexpressing Oy CM. The single and double asterisks indicate p<0.05 and 0.01, respectively. (A) Presence of Dmp1-Cre and floxed Lrp5 in KO mice. Cx43 (connexin 43) was used as a control. (B) Mean age of the mice used in this study. (C&D) Micro-CT images of trabecular bone in the proximal tibia and bone parameters. BMD=bone mineral density, BV/TV=bone volume ratio, Tb.N=trabecular number, and Tb.Sp=trabecular separation. (E) H&E-stained histological images of the proximal tibia. The green dotted area indicates the tumor-invaded region.

FIG. 48 illustrates effects of Lrp5-Oy CM on tumor-suppressing and promoting proteins in TRAMP cells. CM=conditioned medium, Oy=osteocytes, Lrp5=Lrp5 overexpression, shL5=Lrp5 silencing, and pp53=p53 overexpression, (A&B) Elevation of p53, ANXA6, LIMA1, and TRAIL in Lrp5-overexpressing Oy and in Lrp5-Oy CM. (C) Effects of Lrp5-overexpressing and Lrp5-silenced Oy CM on MMP9, Snail, and TGFβ in TRAMP cells. (D&E) Effects of p53 overexpression on p53 and LIMA1 in Oy, and effects of p53-overexpressing Oy CM on MMP9, Snail, and TGFβ in TRAMP cells. (F) Responses of MMP9, Snail, TGFβ, and cleaved caspase 3 (c-cas3) to recombinant TRAIL proteins in TRAMP cells. (G&H) Inhibition of TRANSWELL® invasion of TRAMP cells by Lrp5-overexpressing TRAMP CM. (I) Downregulation of MMP9, Snail, and TGFβ in TRAMP cells in response to Lrp5-overexpressing TRAMP CM.

FIG. 49 illustrates inhibitory effects of Lrp5-Oy CM on osteoclast development and tumor progression. CM=conditioned medium, Oy=osteocytes, Lrp5=Lrp5 overexpression, shL5=Lrp5 silencing, pp53=p53 overexpression, and siTrail=TRAIL silencing. (A) Inhibition of RANKL-driven osteoclast development by Lrp5-Oy CM, and the downregulation of NFATc1 and cathepsin K. The data are presented as mean±S.D. (n=3). (B) Downregulation of NFATc1 and cathepsin K by p53-overexpressing Oy CM. (C) Downregulation of NFATc1 and cathepsin K by recombinant TRAIL proteins, and the partial suppression of Oy CM-driven downregulation by silencing TRAIL. (D) The proposed mechanism of the action of Lrp5 Oy to inhibit tumor progression and osteoclast development.

FIG. 50 illustrates inhibitory effects of Lrp5-overexpressing osteocyte-derived CM on EO771 mammary tumor cells, and MDA-MB-231 breast cancer cells. CN=control, CM=conditioned medium, Oy CM=osteocyte-derived CM, and Lrp5=Lrp5 overexpression. The data are presented as mean±S.D. (n=3). The double asterisks indicate p<0.01, respectively. (A&B) Inhibition of MTT-based viability and the spheroid growth of EO771 mammary tumor cells by Lrp5-Oy CM. (C) Inhibition of scratch-based migration of MDA-MB-231 breast cancer cells by Lrp5-Oy CM.

FIG. 51 illustrates reduction in the weight of mammary tumors by the co-injection of osteocytes (Oy) and Lrp5-overexpressing osteocytes (Lrp5-Oy) to C57BL/6 female mice. The double asterisks indicate p<0.01, respectively.

FIG. 52 illustrates TRAMP-C2ras-invaded tibia samples of C57BL/6 male mice in 4 groups: normal control without tumor inoculation (normal CN), placebo, osteocyte co-injected (Oy), and Lrp5-overexpressing osteocyte co-injected (Lrp5).

FIG. 53 illustrates responses of MDA-MB-231 (MDA), EO771, PC-3, and TRAMP cancer cells to Oy CM with the overexpression of Lrp5 and p53, and the silencing of Lrp5 and TRAIL. CM=conditioned medium, Oy=osteocytes, Oy CM=osteocyte-derived CM, Lrp5=Lrp5 overexpression, shL5=Lrp5 silencing, pp53=p53 overexpression, siTrail=TRAIL silencing, and NC=negative control. (A) Effects of Oy CM, Lrp5-Oy CM, and shL5-Oy CM on MMP9, Snail, and TGFβ in PC-3, MDA-MB-231, and EO771 cells. (B) Effects of Oy CM, and p53-Oy CM on MMP9, Snail, and TGI3 in PC-3, MDA-MB-231, and EO771 cells. (C) Downregulation of p53 and LIMA1 in Oy by TRAIL siRNA, and upregulation of MMP9, Snail, TGFβ, with a decrease in cleaved caspase 3 (c-cas 3) by TRAIL siRNA-treated Oy CM in TRAMP cells and EO771 cells. (D) Tumor-promoting responses by the overexpression of Lrp5 in TRAMP cells. The elevation of EdU-based proliferation, and the upregulation of MMP9, Snail, and TGFβ.

### DETAILED DESCRIPTION

Although the concepts of the present disclosure are susceptible to various modifications and alternative forms, specific embodiments have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should be further appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures unless indicated to the contrary. Additionally, the inclusion of a structural or method feature in a particular figure is not meant to imply that such feature is required in all embodiments and, in some embodiments, may not be included or may be combined with other features.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

The singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

Certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Definitions

“Bone mineral density” refers to the inorganic mineral content in bone.

“Bone volume ratio” refers to the ratio of the segmented bone volume to the total volume.

“Cell growth signaling pathway activator” refers to any substance that enhances or promotes or activates non-cancerous or cancerous mammalian cell growth and/or cell proliferation and/or cell migration activity. Mammalian cell growth signaling pathways include, but are not limited to, highly conserved pathways such as the Wnt signaling pathway, the PI3K signaling pathway, the Fibroblast Growth Factor (FGF) signaling pathway, and the Notch signaling pathway. Cell growth signaling pathway activators useful in the embodiments include small molecules, proteins, fusion proteins, and/or nucleic acids. In embodiments, cell growth signaling pathways include, but are not limited to the Wnt signaling pathway, the OCT3/4 signaling pathway, the PI3K signaling pathway, the Ras-ERK signaling pathway, the Fibroblast Growth Factor (FGF) signaling pathway, the Notch signaling pathway, the c-Myc signaling pathway, and the Epithelial Mesenchymal Transition (EMT) signaling pathway.

“Cell growth signaling pathway inhibitors” refers to any substance that diminishes or inhibits or inactivates non-cancerous or cancerous mammalian cell growth and/or cell proliferation activity. Non-cancerous cells are not treated with cell growth signaling pathway inhibitors in embodiments.

“Wnt signaling pathway” denotes a signaling pathway that may be divided in two pathways: the canonical Wnt/beta catenin signaling pathway and the “Wnt/PCP signaling pathway. Canonical Wnt/beta catenin signaling pathway” or Wnt/PCP signaling pathway denotes a network of proteins and other bioactive molecules (lipids, ions, sugars . . . ) best known for their roles in embryogenesis and cancer, but also involved in normal physiological processes in adult animals. The canonical Wnt/beta catenin signaling pathway is characterized by a Wnt dependant inhibition of glycogen synthase kinase 3f3 (GSK-3(3), leading to a subsequent stabilization of β-catenin, which then translocates to the nucleus to act as a transcription factor. The Wnt/PCP signaling pathway does not involve GSK-30 or β-catenin, and comprises several signaling branches including Calcium dependant signaling, Planar Cell Polarity (PCP) molecules, small GTPases and C-Jun N-terminal kinases (JNK) signaling. These pathways are well known to those skilled in the art.

“Wnt signaling pathway activator” refers to a substance that enhances or promotes or activates a Wnt signaling activity. For example, for the canonical Wnt/β-catenin signaling pathway, this activity can be measured by Wnt reporter activity using established multimers of LEF/TCF binding sites reporters, and/or inhibition of GSK-3β, and/or activation of canonical Wnt target genes such as T, Tbx6, Msgn1, or Axin2. An activation of a Wnt signaling activity may therefore be assessed as being an increase of a Wnt of Msgn1 reporter activity as identified above. The increase may be of at least 1%, 5% 10%, 15%, 20%, 25%, 30%, 35%, 40%, 45%, 50%, 55%, 60%, 65%, 70%, 75%, 80%, 85%, 90%, 95%, 100% or more. Wnt signaling pathway activators are known to those skilled in the art. Small molecule Wnt signaling pathway activators include, but are not limited to, BML-284, CHIR99021, and Wnt pathway activator 1.

“PI3K/Akt signaling pathway activator” refers to a substance that enhances or promotes or activates a PI3K/Akt signaling activity. Small molecule PI3K/Akt signaling pathway activators include, but are not limited to YS-49 and SC79.

“Notch signaling pathway activator” refers to a substance that enhances or promotes or activates a Notch signaling activity. Small molecule Notch signaling pathway activators include, but are not limited to, resveratrol. Small molecule FGF signaling pathway activators, small molecule OCT3/4 signaling pathway activators, small molecule c-Myc signaling pathway activators, small molecule Ras-ERK signaling pathway activators, and small molecule EMT signaling pathway activators are known to those skilled in the art.

Small molecule Oct4 signaling pathway activators include, but are not limited to Oct4 activating compound 2 (OAC2).

“Cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. “Cancer” refers to all types of cancer or neoplasm or malignant tumors found in humans, including, but not limited to: leukemias, lymphomas, melanomas, carcinomas and sarcomas. “Cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell includes not only a primary cancer cell, but also cancer stem cells, as well as cancer progenitor cells or any cell derived from a cancer cell ancestor. This includes metastasized cancer cells, and in vitro cultures and cell lines derived from cancer cells. In certain embodiments, the cancer is a blood tumor (i.e., a non-solid tumor). In some embodiments, the cancer is lymphoid neoplasm diffuse large B-cell lymphoma, cholangiocarcinoma, uterine carcinosarcoma, kidney chromophobe, uveal melanoma, mesothelioma, adrenocortical carcinoma, thymoma, acute myeloid leukemia, testicular germ cell tumor, rectum adenocarcinoma, pancreatic adenocarcinoma, phenochromocytoma and paraganglioma, esophageal carcinoma, sarcoma, kidney renal papillary cell carcinoma, cervical squamous cell carcinoma and endocervical adenocarcinoma, kidney renal clear cell carcinoma, liver hepatocellular carcinoma, glioblastoma multiforme, bladder urothelial carcinoma, colon adenocarcinoma, stomach adenocarcinoma, ovarian serous cystadenocarcinoma, skin cutaneous melanoma, prostate adenocarcinoma, thyroid carcinoma, lung squamous cell carcinoma, head and neck squamous cell carcinoma, brain lower grade glioma, uterine corpus endometrial carcinoma, lung adenocarcinoma, or breast invasive carcinoma. A “solid tumor” is a tumor that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. The tumor does not need to have measurable dimensions.

Most cancers fall within three broad histological classifications: carcinomas, which are the predominant cancers and are cancers of epithelial cells or cells covering the external or internal surfaces of organs, glands, or other body structures (e.g., skin, uterus, lung, breast, prostate, stomach, bowel), and which tend to metastasize; sarcomas, which are derived from connective or supportive tissue (e.g., bone, cartilage, tendons, ligaments, fat, muscle); and hematologic tumors, which are derived from bone marrow and lymphatic tissue. Carcinomas may be adenocarcinomas (which generally develop in organs or glands capable of secretion, such as breast, lung, colon, prostate or bladder) or may be squamous cell carcinomas (which originate in the squamous epithelium and generally develop in most areas of the body). Sarcomas may be osteosarcomas or osteogenic sarcomas (bone), chondrosarcomas (cartilage), leiomyosarcomas (smooth muscle), rhabdomyosarcomas (skeletal muscle), mesothelial sarcomas or mesotheliomas (membranous lining of body cavities), fibrosarcomas (fibrous tissue), angiosarcomas or hemangioendotheliomas (blood vessels), liposarcomas (adipose tissue), gliomas or astrocytomas (neurogenic connective tissue found in the brain), myxosarcomas (primitive embryonic connective tissue), or mesenchymous or mixed mesodermal tumors (mixed connective tissue types). Hematologic tumors may be myelomas, which originate in the plasma cells of bone marrow; leukemias which may be “liquid cancers” and are cancers of the bone marrow and may be myelogenous or granulocytic leukemia (myeloid and granulocytic white blood cells), lymphatic, lymphocytic, or lymphoblastic leukemias (lymphoid and lymphocytic blood cells) or polycythemia vera or erythremia (various blood cell products, but with red cells predominating); or lymphomas, which may be solid tumors and which develop in the glands or nodes of the lymphatic system, and which may be Hodgkin or Non-Hodgkin lymphomas. In addition, mixed type cancers, such as adenosquamous carcinomas, mixed mesodermal tumors, carcinosarcomas, or teratocarcinomas also exist.

Cancers may also be named based on the organ in which they originate i.e., the “primary site,” for example, cancer of the breast, brain, lung, liver, skin, prostate, testicle, bladder, colon and rectum, cervix, uterus, etc. This naming persists even if the cancer metastasizes to another part of the body that is different from the primary site. In accordance with embodiments, treatment is directed to the site of the cancer, not type of cancer, so that a cancer of any type that is situated in the lung, for example, would be treated on the basis of this localization in the lung.

“Triple negative breast cancer (TNBC)” refers to any breast cancer that does not express the genes for estrogen receptor (ER), progesterone receptor (PR) and Her2/neu. The term includes primary epithelial TNBCs, as well as TNBC that involved with other tumors. The cancer can include a triple negative carcinoma of the breast, ductal carcinoma of the breast, lobular carcinoma of the breast, undifferentiated carcinoma of the breast, cystosarcoma phyllodes of the breast, angiosarcoma of the breast, and primary lymphoma of the breast. TNBC can also include any stage of triple negative breast cancer, and can include breast neoplasms having histologic and ultrastructual heterogeneity (e.g., mixed cell types).

“Cell” refers to the basic structural and functional unit of a living organism. In higher organisms, e.g., animals, cells having similar structure and function generally aggregate into “tissues” that perform particular functions. Thus, a tissue includes a collection of similar cells and surrounding intercellular substances, e.g., epithelial tissue, connective tissue, muscle, nerve. An “organ” is a fully differentiated structural and functional unit in a higher organism that may be composed of different types of tissues and is specialized for some particular function, e.g., kidney, heart, brain, liver, etc. Accordingly, by “specific organ, tissue, or cell” is meant herein to include any particular organ, and to include the cells and tissues found in that organ.

“Chemotherapeutic agent” refers to a drug used for the treatment of cancer. Chemotherapeutic agents include, but are not limited to, small molecules, hormones and hormone analogs, and biologics (e.g., antibodies, peptide drugs, nucleic acid drugs). In certain embodiments, chemotherapy does not include hormones and hormone analogs.

“Cancer that is resistant to one or more chemotherapeutic agents” is a cancer that does not respond, or ceases to respond to treatment with a chemotherapeutic regimen, i.e., does not achieve at least stable disease (i.e., stable disease, partial response, or complete response) in the target lesion either during or after completion of the chemotherapeutic regimen. Resistance to one or more chemotherapeutic agents results in, e.g., tumor growth, increased tumor burden, and/or tumor metastasis.

“Conditioned medium” refers to a liquid nutrient medium that has been in contact with and exposed to cultured mammalian cells, where the mammalian cells produce peptides and proteins that enter the media, thus bestowing upon the media a therapeutic activity.

“Disease-free survival” refers to living free of the cancer being monitored. For example, if differential gene expression is used to diagnose or monitor breast cancer, disease-free survival would mean free from detectable breast cancer. In some embodiments, the conditioned medium is selectively toxic towards cancer cells. In other embodiments, the conditioned medium affects cancer cells and healthy cells.

“Epithelial-mesenchymal transition (EMT)” relates to biologic processes that allows a normal or cancer cell to undergo multiple biochemical changes enabling it to assume a mesenchymal cell phenotype, e.g., enhanced migratory capacity, invasiveness, elevated resistance to apoptosis, and greatly increased production of ECM components. Classes of molecules that change in expression, distribution, and/or function during EMT, and that are causally involved, include growth factors (e.g., transforming growth factor-β (TGF-β), wnts), EGF, HGF, transcription factors (e.g., Snail, SMAD, LEF, and nuclear (3-catenin), molecules of the cell-to-cell adhesion axis (cadherins, catenins), cytoskeletal modulators (Rho family), and extracellular proteases (matrix metalloproteinases, plasminogen activators).

“Event-free survival” refers to living without the occurrence of a particular group of defined events (for example progression of cancer) after a particular action (e.g., treatment).

“Mammalian cell culture medium” and “culture medium” (or simply “medium”) refer to a nutrient solution used for growing mammalian cells that typically provides at least one component from one or more of the following categories: (1) salts (e.g., sodium, potassium, magnesium, calcium, etc.) contributing to the osmolality of the medium; (2) an energy source, usually in the form of a carbohydrate such as glucose; (3) all essential amino acids, and usually the basic set of twenty amino acids; (4) vitamins and/or other organic compounds required at low concentrations; and (5) trace elements, where trace elements are defined as inorganic compounds that are typically required at very low concentrations, usually in the micromolar range. The nutrient solution may optionally be supplemented with one or more of the components from any of the following categories: (a) animal serum; (b) hormones and other growth factors such as, for example, insulin, transferrin, and epidermal growth factor; and (c) hydrolysates of plant, yeast, and/or tissues, including protein hydrolysates thereof. Selection of the most appropriate culture medium is within the skill of those in the art.

“Fusion molecule” and “fusion protein” refer interchangeably to a biologically active polypeptide and an effector molecule covalently linked (i.e., fused) by recombinant, chemical or other suitable method. If desired, the fusion molecule can be fused at one or several sites through a peptide linker sequence. Alternatively, the peptide linker may be used to assist in construction of the fusion molecule. In embodiments, fusion molecules are fusion proteins. Generally fusion molecules also can be comprised of conjugate molecules.

“Increased” and grammatical equivalents (including “higher,” “bigger,” etc.) when in reference to the expression of any characteristic in a first subject relative to a second subject, mean that the quantity and/or magnitude of the characteristic in the first subject is greater than in the second subject by any amount that is recognized as clinically relevant by any medically trained personnel. In one embodiment, the quantity and/or magnitude of the characteristic in the first subject is at least 10% greater than, at least 25% greater than, at least 50% greater than, at least 75% greater than, and/or at least 90% greater than the quantity and/or magnitude of the characteristic in the second subject. In embodiments, either the first or second subject may be treated and the other of the first or second subject may be untreated. In embodiments, the first subject is untreated and the second subject is treated.

“Likelihood of reappearance” refers to the probability of tumor reappearance or metastasis in a subject subsequent to diagnosis of cancer.

“Likelihood of recovery” refers to the probability of disappearance of tumor or lack of tumor reappearance resulting in the recovery of the subject subsequent to diagnosis of cancer.

“Metastasis” is well known to one of skill in the art and refers to the growth of a cancerous tumor in an organ or body part, which is not directly connected to the organ of the original cancerous tumor.

“Nucleic acids” and “nucleic acid sequences” refer to oligonucleotide, nucleotide, polynucleotide, or any fragment of any of these; and include DNA or RNA (e.g., mRNA, rRNA, tRNA, iRNA) of genomic or synthetic origin which may be single-stranded or double-stranded; and can be a sense or antisense strand, or a peptide nucleic acid (PNA), or any DNA-like or RNA-like material, natural or synthetic in origin, including, e.g., iRNA, ribonucleoproteins (e.g., e.g., double stranded iRNAs, e.g., iRNPs), nucleic acids, i.e., oligonucleotides, containing known analogues of natural nucleotides.

“Non-cancerous cells” refers to normal cells. Normal cells can be readily distinguished from primary cancer cells and metastatic cancer cells by well-established techniques, particularly histological examination. In embodiments, the non-cancerous cells are protein-secreting cells. Examples of protein-secreting cells include, but are not limited to chondrocytes, adipose derived mesenchymal stem cells, macrophages, pituitary cells, thyroid cells, and pancreatic cells, e.g., pancreatic islet cells. In embodiments, non-cancerous mammalian cells are derived from the same organ or tissue of the same subject as the subject and organ or site to be treated.

“Selective toxicity” is the propensity of an anti-tumor agent or conditioned medium to affect tumor cells in preference to other healthy cells. In some embodiments, the pharmaceutical compositions and conditioned media are selectively toxic towards tumor cells. In other embodiments, the pharmaceutical compositions and conditioned media are not selectively toxic towards tumor cells.

“Overall survival” refers to the fate of a subject after diagnosis, despite the possibility that the cause of death in a subject is not directly due to the effects of the cancer.

“Pharmaceutically acceptable” and “pharmacologically acceptable” refer to compounds and compositions that do not produce adverse, allergic, or other untoward reactions when administered to an animal or a human.

In embodiments, the compositions may be administered to patients in a pharmaceutical composition comprising the conditioned medium (CM) along with a pharmaceutically acceptable carrier. The carrier may be any solvent, diluent, liquid or solid vehicle that is pharmaceutically acceptable and typically used in formulating compositions. Guidance concerning the making of pharmaceutical formulations can be obtained from standard works in the art (see, e.g., Remington's Pharmaceutical Sciences, 16th edition, E. W. Martin, Easton, Pa. (1980)). In addition, pharmaceutical compositions may contain any of the excipients that are commonly used in the art. Examples of carriers or excipients that may be present include, but are not limited to, sugars (e.g., lactose, glucose and sucrose); starches, such as corn starch or potato starch; cellulose and its derivatives (e.g., sodium carboxymethyl cellulose, ethyl cellulose, or cellulose acetate); malt; gelatin; oils (e.g., peanut oil, cottonseed oil, safflower oil, sesame oil, olive oil, corn oil, or soybean oil); glycols; buffering agents; saline; Ringer's solution; alcohols; lubricants; coloring agents; dispersing agents; preservatives; or antioxidants.

“Pharmaceutically acceptable salt” refers to pharmaceutically acceptable organic or inorganic salts of a compound described herein. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Polypeptide” and “protein” refer interchangeably to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. Full-length polypeptides, truncated polypeptides, point mutants, insertion mutants, splice variants, chimeric proteins, and fragments thereof are encompassed by this definition. In various embodiments the polypeptides can have at least 10 amino acids or at least 25, or at least 50 or at least 75 or at least 100 or at least 125 or at least 150 or at least 175 or at least 200 amino acids.

“Progression-free survival” is well known to one of skill in the art and refers to the length of time during and after treatment in which a subject is living with a cancer that does not get worse, and can be used in a clinical study or trial to help find out how well a treatment is working.

“Reduced” and grammatical equivalents (including “lower,” “smaller,” etc.) when in reference to the expression of any characteristic in a first subject relative to a second subject, mean that the quantity and/or magnitude of the characteristic in the first subject is lower than in the second subject by any amount that is recognized as clinically relevant by any medically trained personnel. In one embodiment, the quantity and/or magnitude of the characteristic in the first subject is at least 10% lower than, at least 25% lower than, at least 50% lower than, at least 75% lower than, and/or at least 90% lower than the quantity and/or magnitude of the characteristic in the second subject. In embodiments, either the first or second subject may be treated and the other of the first or second subject may be untreated. In embodiments, the first subject is treated and the second subject is untreated.

“Small molecule” refers to a low molecular weight organic compound that may help regulate a biological process. Small molecules include any molecules with a molecular weight of about 2000 daltons or less, such as of about 500 to about 900 daltons or less. Small molecules can have a variety of biological functions, serving as cell signaling molecules, as drugs in medicine, and in many other roles. These compounds can be natural or artificial. Biopolymers such as nucleic acids and proteins, and polysaccharides (such as starch or cellulose) are not small molecules—though their constituent monomers, ribo- or deoxyribonucleotides, amino acids, and monosaccharides, respectively, are often considered small molecules. Small molecules include pharmaceutically acceptable salts of small molecules.

“Subject” refers to any mammal for whom diagnosis, treatment, or therapy is desired including mammals, e.g., humans, laboratory animals (e.g., primates, rats, mice, rabbits), livestock (e.g., cows, sheep, goats, pigs, turkeys, and chickens), household pets (e.g., dogs, cats, and rodents), and horses.

“Substantially homogenous” refers to a population of cells derived from the same mammalian organ or region of a mammalian organ wherein the majority between about 100% to about 70%; between about 100% to about 90% of the total number of cells have a specified characteristic of interest

“Therapeutically effective amount” is that amount sufficient, at dosages and for periods of time necessary, to achieve a desired therapeutic result, such as for treatment of a disease (e.g. cancer), condition, or disorder, and/or pharmacokinetic or pharmacodynamic effect of the treatment in a subject. A therapeutically effective amount can be administered in one or more administrations. The therapeutically effective amount may vary according to factors such as the disease state, age, sex, and weight of the subject. One skilled in the art will recognize that the condition of the individual can be monitored throughout the course of therapy and that the effective amount of a compound or composition disclosed herein that is administered can be adjusted accordingly.

“Trabecular number” refers to the average number of trabeculae per unit length.

“Trabecular separation” refers to the mean distance between trabeculae.

“Treat,” “treating” or “treatment” refer to an action to obtain a beneficial or desired clinical result including, but not limited to, alleviation or amelioration of one or more signs or symptoms of a disease or condition (e.g., regression, partial or complete), diminishing the extent of disease, stability (i.e., not worsening, achieving stable disease) of the state of disease, amelioration or palliation of the disease state, diminishing rate of or time to progression, and remission (whether partial or total). “Treatment” of a cancer can also mean prolonging survival as compared to expected survival in the absence of treatment. Treatment need not be curative. In certain embodiments, treatment includes one or more of a decrease in pain or an increase in the quality of life (QOL) as judged by a qualified individual, e.g., a treating physician, e.g., using accepted assessment tools of pain and QOL. In certain embodiments, a decrease in pain or an increase in the QOL as judged by a qualified individual, e.g., a treating physician, e.g., using accepted assessment tools of pain and QOL is not considered to be a “treatment” of the cancer. “Treat” covers any treatment of a cancer in a mammal, and includes: (a) preventing the cancer from occurring in a subject which may be predisposed to the disease but has not yet been diagnosed as having it; (b) inhibiting the cancer, i.e., arresting its development; or (c) relieving the cancer, i.e., causing regression of the cancer. The therapeutic composition may be administered before, during or after the onset of cancer. The therapy described herein may be administered during the symptomatic stage of the cancer, and in some cases after the symptomatic stage of the cancer.

“Tumor microenvironment” or “cancer microenvironment” refers to the cellular environment or milieu in which the tumor or neoplasm exists, including surrounding blood vessels as well as non-cancerous cells including, but not limited to, immune cells, fibroblasts, bone marrow-derived inflammatory cells, and lymphocytes. Signaling molecules and the extracellular matrix also comprise the tumor microenvironment. The tumor and the surrounding microenvironment are closely related and interact constantly. Tumors can influence the microenvironment by releasing extracellular signals, promoting tumor angiogenesis and inducing peripheral immune tolerance, while the immune cells in the microenvironment can affect the growth and evolution of tumor cells.

Aspects of the present disclosure can be described as embodiments in any of the following enumerated clauses. It will be understood that any of the described embodiments can be used in connection with any other described embodiments to the extent that the embodiments do not contradict one another.

Clause. A pharmaceutical composition comprising a cell-free conditioned medium (CM) or extract or concentrate thereof obtained from a mammalian cell culture medium comprising a cultured substantially homogenous non-cancerous mammalian cell population where a portion of the non-cancerous mammalian cell population is contacted by a small molecule cell growth signaling pathway activator before being cultured in the cell culture medium.

Clause. A pharmaceutical composition according to the preceding clause, where the conditioned medium is concentrated.

Clause. A pharmaceutical composition according to any of the preceding clauses, where the a portion of the non-cancerous mammalian cell population is contacted by at least two small molecule cell growth signaling pathway activators before being cultured in the cell culture medium.

Clause. A pharmaceutical composition according to any of the preceding clauses, further comprising a pharmaceutically acceptable carrier.

Clause. A pharmaceutical composition according to any of the preceding clauses, where the conditioned medium further comprises a non-cancerous mammalian cell-secreted protein selected from the group consisting of Hsp90ab1, Calr, Ppia, Ppib, Flna, H4, Vim, and Ubc.

Clause. A pharmaceutical composition according to any of the preceding clauses, where the composition is enriched with a non-cancerous mammalian cell-secreted protein selected from the group consisting of heat shock protein 90 alpha family class B member 1 (Hsp90ab1), calreticulin (Calr), peptidylprolyl isomerase A (Ppia), peptidylprolyl isomerase B (Ppib), filamin A (Flna), histone H4 (H4), vimentin (Vim), and ubiquitin C (Ubc).

Clause. A pharmaceutical composition according to any of the preceding clauses, further comprising a chemotherapeutic agent.

Clause. A pharmaceutical composition according to any of the preceding clauses, where the small molecule cell growth signaling pathway activator is a small molecule Wnt signaling pathway activator.

Clause. A pharmaceutical composition according to any of the preceding clauses, where the small molecule Wnt signaling pathway activator is BML-284, or a pharmaceutically acceptable salt thereof.

Clause. A pharmaceutical composition according to any of the preceding clauses, where the non-cancerous mammalian cells are non-cancerous mammalian bone cells.

Clause. A pharmaceutical composition according to any of the preceding clauses, where the non-cancerous mammalian bone cells are osteocytes.

Clause. A pharmaceutical composition according to any of the preceding clauses, where the non-cancerous mammalian cells are non-cancerous mammalian bone cells isolated from bone marrow.

Clause. A kit comprising: a) a pharmaceutical composition according to any of the preceding clauses; b) a container; c) a label; and d) instructions that provide methods for administering the composition.

Clause. A kit according to the preceding clause, where the pharmaceutical composition further comprises at least one preservative.

Clause. A method to treat a cancer in a subject in need thereof, the method comprising: administering to the subject in need thereof a therapeutically effective amount of a cell-free conditioned medium (CM) or extract or concentrate thereof obtained from a mammalian cell culture medium comprising a cultured substantially homogenous non-cancerous mammalian cell population where a portion of the non-cancerous mammalian cell population is contacted by a small molecule cell growth signaling pathway activator before being cultured in the cell culture medium.

Clause. A method according to the preceding clause, where the subject is a human.

Clause. A method according to any of the preceding clauses, where the treated cancer is a metastatic cancer and the cultured substantially homogenous non-cancerous mammalian cell population is derived from a same organ or tissue as the organ or site to be treated.

Clause. A method according to any of the preceding clause where the treated cancer is a metastatic cancer and the cultured substantially homogenous non-cancerous mammalian cell population is derived from the same subject as the subject to be treated.

Clause. A method according to any of the preceding clauses, where the treated cancer is a metastatic cancer selected from the group consisting of metastatic bone cancer, metastatic liver cancer, metastatic lung cancer, and metastatic brain cancer.

Clause. A method according to any of the preceding clauses, where the treated cancer is a metastatic bone cancer.

Clause. A method according to any of the preceding clauses, where the treated cancer is a primary cancer.

Clause. A method according to any of the preceding clauses, where the treated cancer is a primary cancer selected from the group consisting of breast cancer, lung cancer, colorectal cancer, prostate cancer, skin cancer, and pancreatic cancer.

Clause. A method according to any of the preceding clauses, where the treated primary cancer is breast cancer selected from the group consisting of Estrogen Receptor (ER)-positive breast cancer, Estrogen Receptor (ER)-negative breast cancer, and triple-negative breast cancer.

Clause. A method according to any of the preceding clauses, where the non-cancerous mammalian cell population is non-cancerous mammalian bone cells selected from the group consisting of osteocytes, bone marrow-derived mesenchymal stem cells, and osteoblasts.

Clause. A method according to any of the preceding clauses, where the small molecule cell growth signaling pathway activator is selected from the group consisting of a small molecule Wnt signaling pathway activator, a small molecule PI3K signaling pathway activator, a small molecule FGF signaling pathway activator and a small molecule Notch signaling pathway activator.

Clause. A method according to any of the preceding clauses, where the small molecule cell growth signaling pathway activator is a small molecule Wnt signaling pathway activator.

Clause. A method according to any of the preceding clauses, where the small molecule Wnt signaling pathway activator is BML-284, or a pharmaceutically acceptable salt thereof.

Clause. A method according to any of the preceding clauses, where the cancer is metastatic bone cancer and the treatment reduces cancer-induced osteolysis.

Clause. A method according to any of the preceding clauses, where the cancer is breast cancer and the treatment reduces mammary tumor size.

Clause. A method according to any of the preceding clauses, where the cancer is metastatic bone cancer and the treatment increases bone volume ratio.

Clause. A method according to any of the preceding clauses, where the cancer is metastatic bone cancer and the treatment increases bone mineral density.

Clause. A method according to any of the preceding clauses, where the cancer is metastatic bone cancer and the treatment increases trabecular number.

Clause. A method according to any of the preceding clauses, where the cancer is metastatic bone cancer and the treatment reduces trabecular separation.

Clause. A method according to any of the preceding clauses, where the cancer is metastatic bone cancer and the treatment reduced osteoclastogenesis.

Clause. A method according to any of the preceding clauses, where the conditioned medium is in a pharmaceutical form suitable for systemic administration.

Clause. A method according to any of the preceding clauses, where the conditioned medium is in a pharmaceutical form suitable for local administration.

Clause. A method according to any of the preceding clauses, where the conditioned medium is in a pharmaceutical form suitable for administration by injection.

Clause. A method according to any of the preceding clauses, further comprising administering a chemotherapeutic agent.

Clause. A method according to any of the preceding clauses where apoptosis is not induced in normal cells.

Clause. A method according to any of the preceding clauses, where the method results in at least one activity selected from the group consisting of upregulating a tumor-suppressing gene in a cancer cell, downregulating a tumor-promoting gene in a cancer cell, inhibiting cancer cell invasion, inhibiting cancer cell growth and inhibiting cancer cell recurrence.

Clause. A method according to any of the preceding clauses, where the target cancer is a therapy-resistant cancer

Clause. A process to produce a conditioned medium (CM), the process comprising: contacting non-cancerous mammalian cells by a small molecule cell growth signaling pathway activator to generate pre-treated non-cancerous mammalian cells; culturing the pre-treated non-cancerous mammalian cells in a mammalian cell culture medium for a period of time sufficient to condition the medium; removing the pre-treated non-cancerous mammalian cells from the culture medium; and, collecting the conditioned medium.

Clause. A process according to the preceding clause, where the collected conditioned medium comprises a non-cancerous mammalian cell-secreted protein selected from the group consisting of Hsp90ab1, Calr, Ppia, Ppib, Flna, H4, Vim, and Ubc.

Clause. A process according to any of the preceding clauses, where the mammalian cell culture medium is serum-free.

Clause. A process according to any of the preceding clauses, where the pre-treated non-cancerous mammalian cells are cultured for a time period from about 1 hour to about 4 hours.

Clause. A process according to any of the preceding clauses, further comprising filtering the collected conditioned medium.

Clause. A process according to any of the preceding clauses, further comprising ultra-filtering the collected conditioned medium.

Clause. A process according to any of the preceding clauses, further comprising concentrating the collected conditioned medium.

Clause. A process according to any of the preceding clauses, further comprising purifying the collected conditioned medium.

Clause. A method to identify an anti-tumor property in a conditioned medium (CM), the method comprising: contacting non-cancerous mammalian cells by a small molecule cell growth signaling pathway activator to generate pre-treated non-cancerous mammalian cells; culturing the pre-treated non-cancerous mammalian cells in a mammalian cell culture medium to condition the medium; removing the pre-treated non-cancerous mammalian cells from the culture medium; collecting the conditioned medium; and, assaying the conditioned medium for an anti-tumor property.

Clause. A method to treat cancer in a subject in need thereof, comprising administering to the subject an effective amount of heat shock protein 90 alpha family class B member 1 (Hsp90ab1).

Clause. A method to treat cancer in a subject in need thereof, comprising administering to the subject an effective amount of calreticulin (Calr).

Clause. A method to treat cancer in a subject in need thereof, comprising administering to the subject an effective amount of peptidylprolyl isomerase A (Ppia).

Clause. A method to treat cancer in a subject in need thereof, comprising administering to the subject an effective amount of peptidylprolyl isomerase B (Ppib).

Clause. A method to treat cancer in a subject in need thereof, comprising administering to the subject an effective amount of filamin A (Flna).

Clause. A method to treat cancer in a subject in need thereof, comprising administering to the subject an effective amount of histone H4 (H4).

Clause. A method to treat cancer in a subject in need thereof, comprising administering to the subject an effective amount of vimentin (Vim).

Clause. A method to treat cancer in a subject in need thereof, comprising administering to the subject an effective amount of ubiquitin C (Ubc).

### EXAMPLES

Examples related to the present disclosure are described below. In some embodiments, alternative techniques can be used. The examples are intended to be illustrative and are not limiting or restrictive of the scope of the invention as set forth in the claims.

### Example 1

Osteocytes Cell culture. EO771 mouse mammary tumor cells (CH3 BioSystems, Amherst, NY, USA), 4T1.2 mouse mammary tumor cells (obtained from Dr. R. Anderson at Peter MacCallum Cancer Institute, Melbourne, Australia), and fibroblast cells (CRL3063; ATCC, Manassas, VA, USA) were cultured in Dulbecco's Modified Eagle Medium (“DMEM”). MBA-MB-231 breast cancer cells (ATCC), MLO-A5 and MLO-Y4 osteocyte-like cells (C57BL/6 background; obtained from Dr. L. Bonewald at Indiana University, IN, USA), and RAW264.7 pre-osteoclast cells (ATCC) were grown in Minimum Essential Medium Eagle—a Modification (“aMEM”). Human primary osteocytes (Celprogen, 36043-15) were maintained in the human osteocyte primary cell culture complete growth medium (Celprogen, M36043-155) and sub-cultured on the extracellular matrix (Celprogen, E36043-15). PC-3 human prostate cancer cells (ATCC) were cultured in Roswell Park Memorial Institute 1640 Medium (“RPMI-1640”) (Gibco, Carlsbad, CA, USA). Primary human breast cancer cells (ER+/PR-0514-15, and triple-negative 0514-21) were grown by methods known to those skilled in the art. The culture media was supplemented with 10% fetal bovine serum and antibiotics (100 units/ml penicillin, and 100 μg/ml streptomycin; Life Technologies, Grand Island, NY, USA), and cells were maintained at 37° C. and 5% CO₂. Plasmids for Lrp5 (40 ng/μ1) and β-catenin (40 ng/μ1) were transfected to 2×10⁶ osteocytes overnight. After one-day incubation, the conditioned medium was ultra-centrifuged to remove exosomes and condensed 10-fold by filtering (Amicon, Sigma, Saint Louis, MO, USA) with a cutoff molecular weight at 3 kDa. Proteins from CM-treated cells were harvested 24 h after the onset of incubation.

EdU assay. Approximately 2,000 cells were seeded in 96-well plates on day 1. CM was added on day 2 and cellular proliferation was examined using a fluorescence-based cell proliferation kit (CLICK-IT™ EDU ALEXA FLUOR™ 488 Imaging Kit; Thermo-Fisher, Waltham, MA, USA) on day 4. After fluorescent labeling, the number of fluorescently labeled cells was counted and the ratio to the total number of cells was determined.

TRANSWELL® invasion assay. The invasion capacity of cancer cells was determined using a 24-well plate, TRANSWELL® chambers (Thermo Fisher Scientific, Waltham, MA, USA) with 8-mm pore size, and MATRIGEL® (100 jag/ml), a solubilized basement membrane matrix secreted by Engelbreth-Holm-Swarm mouse sarcoma cells. Approximately 5×10⁴ cells in 200 μL serum-free DMEM were placed on the upper chamber and 800 μL of CM were added in the lower chamber. After 48 h, the cells that had invaded the lower side of the membrane were stained with Crystal Violet. At least five randomly chosen images were taken, and the average number of stained cells was determined.

Two-dimensional motility assay. A wound-healing scratch motility assay was performed to evaluate 2-dimensional cell motility. Approximately 4×10⁵ cells were seeded in 12-well plates. After cell attachment, a plastic pipette tip was used to scratch a gap on the cell layer. Floating cells were removed, and CM was added. Images of the cell-free scratch zone were obtained via an inverted microscope at 0 h, and the areas newly occupied with cells were determined 24-48 h after scratching. The areas were quantified with IMAGE J (National Institutes of Health, Bethesda, MD, USA).

Osteoclast differentiation assay. Using RAW264.7 pre-osteoclast cells, an osteoclast differentiation assay was conducted in 12-well plates. During the 6-day incubation of pre-osteoclast cells in 40 ng/ml of RANKL, the culture medium was exchanged once on day 4. Adherent cells were fixed and stained with a tartrate-resistant acid phosphate (TRAP)-staining kit (Sigma-Aldrich, Missouri, USA), according to the manufacturer's instructions. TRAP-positive multinucleated cells (>3 nuclei) were identified as mature osteoclasts and counted.

Western blot analysis and mass spectrometry. Western blot analysis was conducted using known procedures. Antibodies were used against ANXA1, β-catenin, caspase 3, Lrp5, Lrp6, Runx2, Sclerostin, Snail, TGFβ, NFATc1, cathepsin K (Cell Signaling, Danvers, MA, USA), DMP1, ANXA6, CXCL5 (Abcam, Cambridge, MA, USA), M-CSF, MMP9, OPN, TPM4 (Santa Cruz, Dallas, TX, USA), WISP1 (R&D systems, Minneapolis, MN, USA), β-actin (Sigma, Saint Louis, MO, USA), LIMA1, Trail (Novus, Centennial, CO, USA), p53 and CXCL1 (Invitrogen, Carlsbad, California, USA), and DSP (ProteinTech, Rosemont, IL, USA). The expression levels of Sclerostin and Lrp5 in CM were detected by ELISA (My BioSource, San Diego, CA, USA). Proteins from A5 osteocyte CM, Y4 osteocyte CM, and osteoclast control CM (RAW264.7 cells) were analyzed by the HF Hybrid Quadrupole Orbitrap mass spectrometry. Among 549 identified proteins, 49 proteins presented higher expression levels in A5 CM than Y4 CM and control CM. Among them, 11 proteins (p53; SPARC=osteonectin; TPM1, TPM4=tropomyosin 1 & 4; ANXA1, ANXA6=annexin A1 & A6; FMOD=fibromodulin; OGN=osteoglycin; DSP=desmoplakin; AHNAK=desmoyokin; and LIMA1=LEVI domain actin-binding protein 1) were identified as potential tumor suppressors.

Plasmid transfection, RNA interference, and cytokine analysis. For overexpressing Lrp5 (#115907, Addgene, Watertown, MA, USA) or β-catenin (#31785, Addgene), A5 osteocytes or EO771 tumor cells were transfected with plasmids consisting of their coding sequence, while a blank plasmid vector (FLAG-HA-pcDNA3.1; Addgene) was used as a control. A5 cells or EO771 cells were also treated with shRNA specific to Lrp5 (sc-149050-V, Santa Cruz), Lrp6 (sc-37234-V, Santa Cruz), and Runx2 (sc-37146-V, Santa Cruz), with control GFP shRNA (sc-108084, Santa Cruz). Cells were grown in a 10 cm-plate and transfected with β-catenin plasmids, or control plasmids using LIPOFECTAMINE®3000 (Thermo, L300015). First, plasmids/shRNA were diluted in 200 μL Opti-MEM, and 2 μL of P3000 was added for 1 μg of DNA/shRNA. Then, 20 μL of LIPOFECTAMINE® 3000 was mixed with 200 μL Opti-MEM. The transfection was performed overnight, and stable transfectants for shRNA were selected using puromycin (Sigma). Besides shRNA, siRNAs were employed for β-catenin, and Trail, together with a nonspecific negative control siRNA (SILENCER SELECT™ #1, Life Technologies; ON-TARGET PLUS™ Non-targeting Pool, Dharmacon). Cells were transiently transfected with siRNA with LIPOFECTAMINE® RNAiMAX (Life Technologies). Twenty-four hours later, the medium was replaced by a regular culture medium. The efficiency of silencing was assessed with immunoblotting 24 h after transfection. A mouse XL cytokine array (R&D Systems) was employed and the expression of 111 cytokines and chemokines in osteocyte-derived CM was determined.

3D spheroid assay and ex vivo tissue assay. Cells were cultured in Ultra-low attachment 96-well plates (S-BIO, New Hampshire, USA) at 1×10⁴ cells/well for EO771 cells, and 5×10³ cells/well for A5 cells. Cells were imaged every 24 h, and the area was calculated with IMAGE J. In the ex vivo tissue assay, the usage of human breast cancer tissues was approved by the Indiana University Institutional Review Board. A sample (˜1 g; ER/PR+, HER2+), received from Simon Cancer Center Tissue Procurement Core, was manually fragmented with a scalpel into small pieces (0.5˜0.8 mm in length). These pieces were grown in DMEM with 10% fetal bovine serum and antibiotics for a day. Osteocyte-derived CM was then added for two additional days, and a change in the fragment size was determined.

FRET imaging. To evaluate tension force at a focal adhesion and migratory capacity of tumor cells in response to A5 CM and Runx2 shRNA treatment, a plasmid expressing a vinculin tension sensor (VinTS, #26019, Addgene) was transfected. The fluorescence lifetime images were acquired by a custom-made microscope built on a laser scanning confocal microscope (FLUOVIEW™ 1000, Olympus; Center Valley, PA, USA), using the procedures known to those in the art. A picosecond-pulsed laser with the wavelength of 450 nm was coupled with the laser-scanning module. All signals were recorded in the time-correlated single photon-counting mode with a data acquisition board (TimeHarp 260, Picoquant; Berlin, Germany). The FRET efficiency of the TS module was calculated based on the lifetime of the donor molecule. Of note, an elevation in the tension force of the vinculin sensor implies an increase in fluorescence lifetime.

Animal models. The experimental procedures using animals were approved by the Indiana University Animal Care and Use Committee and were complied with the Guiding Principles in the Care and Use of Animals endorsed by the American Physiological Society. C57BL/6 mice lacking Lrp5 in osteocytes (Dmp1-Cre; Lrp5f/f) were created by breeding Dmp1-Cre transgenic mice with Lrp5 floxed mice. Mice were housed five per cage and provided with mouse chow and water ad libitum. In the mouse model of a mammary tumor, C57BL/6 female mice (˜8 weeks, Envigo RMS, Inc., Indianapolis, IN, USA) and NOD/Scid mice (˜8 weeks, The Jackson Laboratory, Bar Harbor, ME, USA) received subcutaneous injections of EO771 cells and MDA-MB-231 cells (3.0×10⁵ cells in 50 μl phosphate-buffered saline; “PBS”), respectively, to the mammary fat pad on day 1. A5 osteocytes (1.5×10⁵ cells) for the treatment group were co-injected with EO777 cells or MDA-MB-231 cells on day 1, and osteocyte-derived CM was injected into the intraperitoneal cavity from day 2 to day 18. The animals were sacrificed on day 18, and the weight of each tumor was measured. In the mouse model of osteolysis, ten C57BL/6 female mice per group received EO771 cells (3.0×10⁵ cells in 20 μl PBS) to the right tibia as an intra-tibial injection on day 1. A5 osteocytes (1.5×10⁵ cells) were co-injected with EO771 cells into the proximal tibia on day 1. The intramuscular injection of osteocyte-derived CM to the proximal tibia was conducted from day 2 to day 18. EO771 cells were transfected with shRNA specific to Lrp5, Lrp6, or Runx2. Osteocytes were transfected with shRNA specific to Lrp5 or Lrp6, and plasmids for Lrp5 or J3-catenin.

To evaluate the effects of CMs for tumor invasion, the in vivo extravasation assay was conducted. C57BL/6 female mice (5 mice per group) received an injection of 50 μl of fluorescently labeled EO771 cells (1.0×10⁶ cells) into the lateral tail veins. Fluorescently labeled EO771 cells were prepared by culturing them with a green fluorescent dye (#4705, Sartorius, Gottingen, Germany) for 20 min at 37° C. Cells were then centrifuged with 1000 rpm for 5 min for harvesting the pellet. The pellet was re-suspended in PBS (placebo group) or osteocyte-derived CM (A5 CM group). Mice will be sacrificed after 48 h for histologically identifying extravascular tumor cells in the lung.

X-ray and MR imaging. A whole-body X-ray image was taken using the Faxitron radiographic system (Faxitron X-ray Co., Tucson, AZ, USA). The tibia integrity was scored blindly at the levels of 0 to 3, in which level 0=normal with no tumor sign; level 1=clear bone boundary with slight periosteum proliferation; level 2=bone damage and moderate periosteum proliferation; and level 3=severe bone erosion. MR imaging was conducted with a Bruker 7T 70/30 USR system (Bruker BioSpin Co., Billerica, MA, USA). Rhe Turbo RARE sequence was employed for high resolution (T2-weighted imaging) with the Bruker interface (Paravision V6.0.2).

μCT imaging and histology. The tibia was harvested for μCT imaging and histology. Micro-computed tomography was performed using SKYSCAN™ 1172 (Bruker-MicroCT, Kontich, Belgium). Using manufacturer-provided software, scans were performed at pixel size 8.99 μm and the images were reconstructed (nRecon v1.6.9.18) and analyzed (CTan v1.13). In histology, H&E staining was conducted as described previously, and immunohistochemistry was performed using the procedure previously described.

Statistical analysis. For cell-based experiments, three or four independent experiments were conducted, and data were expressed as mean±S.D. In animal experiments, the sample size in the mouse model was chosen to achieve a power of 80% with p<0.05. The primary experimental outcome was tumor weight for the mammary fat pad experiment and the bone volume ratio (BV/TV) for the tibia experiment. The secondary experimental outcome was tumor size for the mammary fat pad experiment and the trabecular number (Tb.n) for the tibia experiment. Statistical significance was evaluated using a one-way analysis of variance (ANOVA). Post hoc statistical comparisons with control groups were performed using Bonferroni correction with statistical significance at p<0.05. A nonparametric Kolmogorov-Smirnov test was applied to compare cell aspect ratios. The single and double asterisks in the figures indicate p<0.05 and p<0.01, respectively.

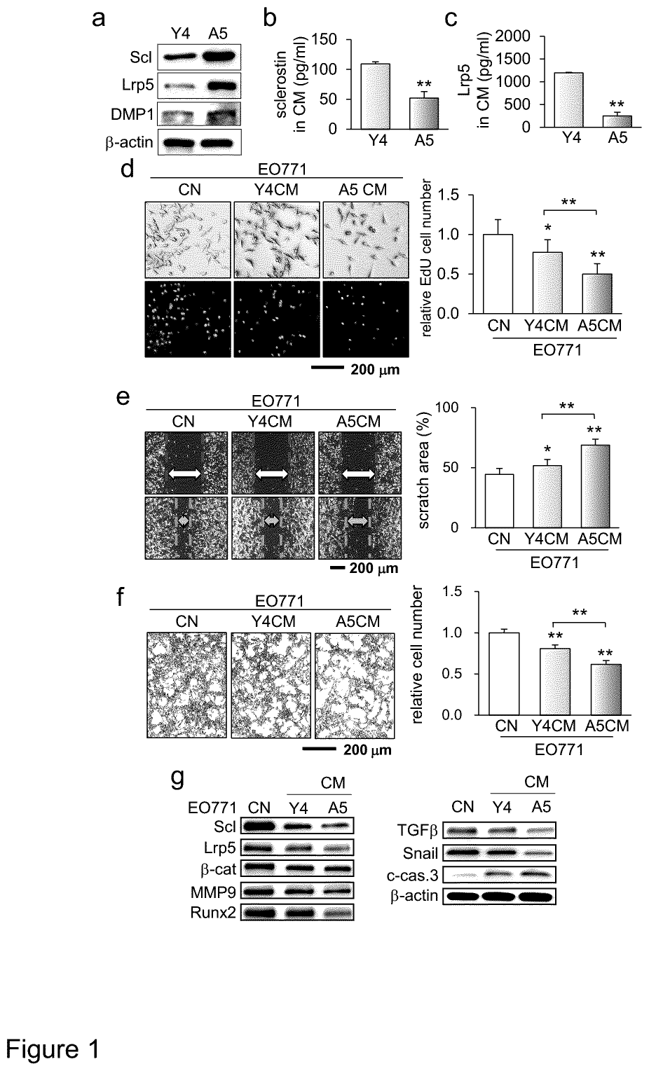

Differentiated osteocytes inhibited the proliferation, migration, and invasion of mammary tumor cells. To evaluate the role of osteocytes in cancer progression, MLO-A5 pre-osteocytes were differentiated by treating them with ascorbic acid. Compared to MLO-Y4 osteocyte-like cells that are premature osteocytes, the ascorbic acid-treated A5 osteocytes expressed elevated levels of Sclerostin (Scl), a marker of osteocyte differentiation, Lrp5, a co-receptor for Wnt signaling, and DMP1, a matrix protein involved in bone mineralization (FIG. 1a). By contrast, it was observed that A5 osteocyte-derived CM contained lower levels of Sclerostin and Lrp5 than Y4 osteocyte-derived CM (FIGS. 1B and 1c). Compared to Y4 CM, A5 CM presented a stronger inhibiting effect on the EdU-based proliferation, scratch-based migration, and TRANSWELL® invasion of EO771 mammary tumor cells (FIGS. 1d-1f). Furthermore, the expression of tumorigenic genes such as MMP9, Runx2, TGFβ, and Snail that play critical roles in the progression and metastasis of breast cancer were evaluated. Western blot results showed that A5 CM downregulated tumor-promoting genes such as Sclerostin, Lrp5, β-catenin, MMP9, Runx2, TGFβ, and Snail and elevated an apoptosis marker, cleaved caspase3, in EO771 cells more substantially than Y4 CM (FIG. 1g).

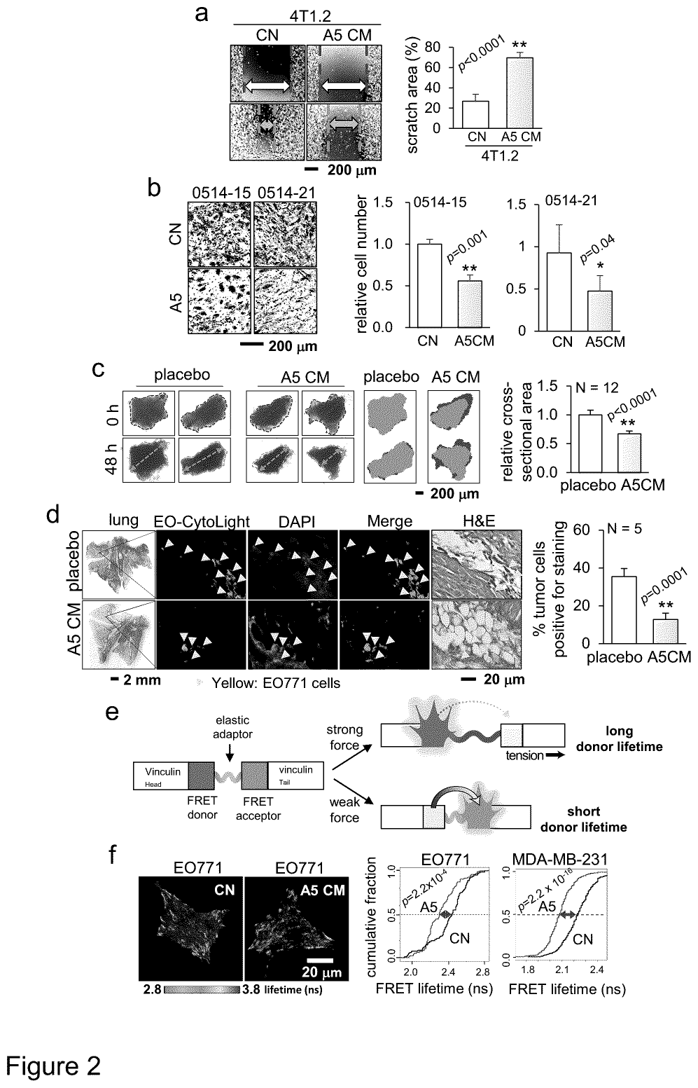

Differentiated osteocytes inhibited the migration and invasion in vitro, ex vivo, and in vivo. The effects of CM from differentiated osteocytes on the migratory and invasive properties of mammary tumor cells were determined. A5 CM reduced scratch-based cellular migration of 4T1.2 mammary tumor cells (FIG. 2a) and TRANSWELL® invasion of primary human breast cancer cells (FIG. 2b). Of note, 0514-15 cells were derived from pleural effusion of ER+/PR-breast cancer, whereas 0514-21 cells were derived from chest wall metastasis of triple-negative breast cancer. A5 CM also inhibited ex vivo growth of human breast cancer tissue fragments (FIG. 2c) and reduced the number of colonized tumor cells in the mouse lung in the in vivo extravasation assay (FIG. 2d). To examine the effect of osteocytes at the biophysical level, a vinculin tension sensor was emloyed. The vinculin head and tail were linked to a FRET donor and acceptor (FIG. 2e). The fluorescence lifetime of the FRET donor was determined. Compared to the control medium, A5 CM shortened FRET donor lifetime in EO771 and MDA-MB-231 cells (FIG. 2f), indicating that A5 CM weakened tensile forces at focal adhesions.

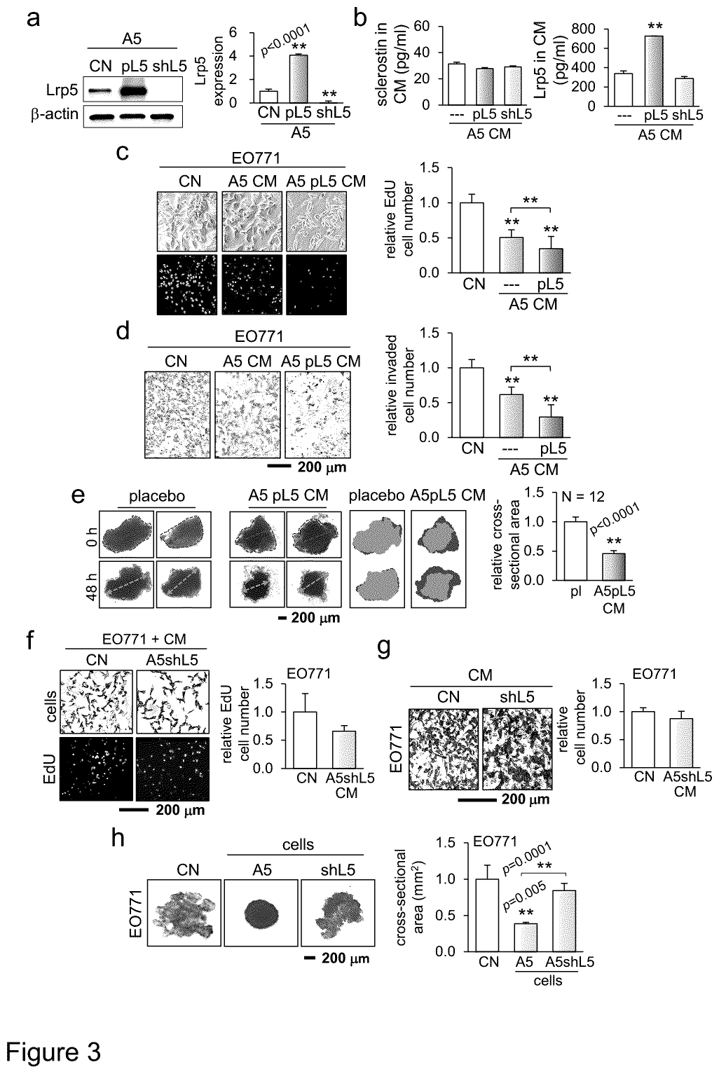

Lrp5, expressed in osteocytes, enhanced anti-tumor capability in vitro. The results so far suggested the anti-tumor action of osteocytes. Since osteocytes are known to regulate Wnt signaling, it was examined whether the overexpression of Lrp5 in osteocytes alters the anti-tumor capability (FIG. 3a). The overexpression of Lrp5 in A5 osteocytes elevated the level of Lrp5 in A5 osteocytes and their CM, while the silencing of Lrp5 reduced its level in A5 osteocytes (FIGS. 3a and 3b). Of note, the level of Sclerostin in A5 CM did not alter by the overexpression and silencing of Lrp5 (FIG. 3b). It was observed that Lrp5-overexpressing osteocyte-derived CM inhibited the EdU-based proliferation and TRANSWELL® invasion of EO771 tumor cells (FIGS. 3C and 3d). Furthermore, Lrp5-overexpressing osteocyte-derived CM significantly inhibited the growth of ex vivo cancer tissue fragments (FIG. 3e). By contrast, Lrp5-silenced osteocyte-derived CM lost the ability to inhibit cellular proliferation and invasion (FIGS. 3f and 3g), as well as the growth of tumor spheroids (FIG. 3h). Collectively, the results indicated that Lrp5 in osteocytes was in part responsible for their anti-tumor actions.

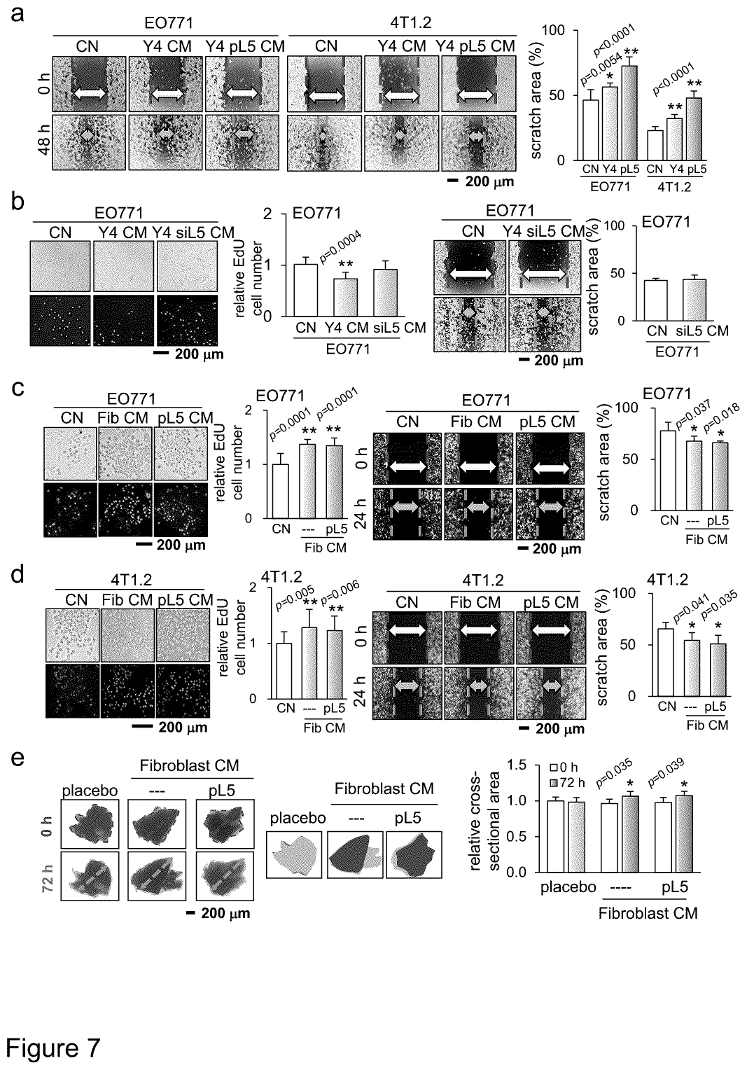

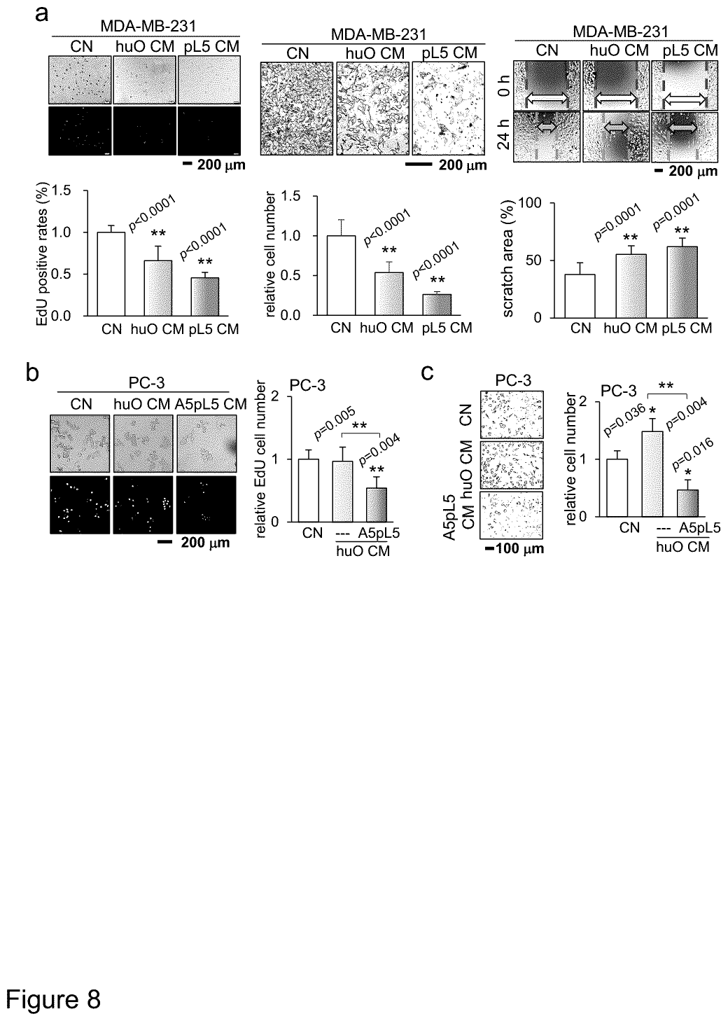

The enhancement of anti-tumor effects by the overexpression of Lrp5 was also observed in Y4 CM (FIGS. 7a and 7b). However, this anti-tumor function of Lrp5 was not observed in fibroblast-derived CM (FIGS. 7c-7e). Human osteocyte-derived CM with and without Lrp5 overexpression also inhibited the proliferation, invasion, and migration of tumor cells (FIG. 8a). Furthermore, Lrp5-overexpressing but not parental osteocyte-derived CM reduced proliferation and invasive properties of PC-3 prostate cancer cells (FIGS. 2b and 2c). Taken together, Lrp5-overexpressing mouse or human osteocytes acted as a tumor suppressor not only in breast cancer cells but also in prostate cancer cells.

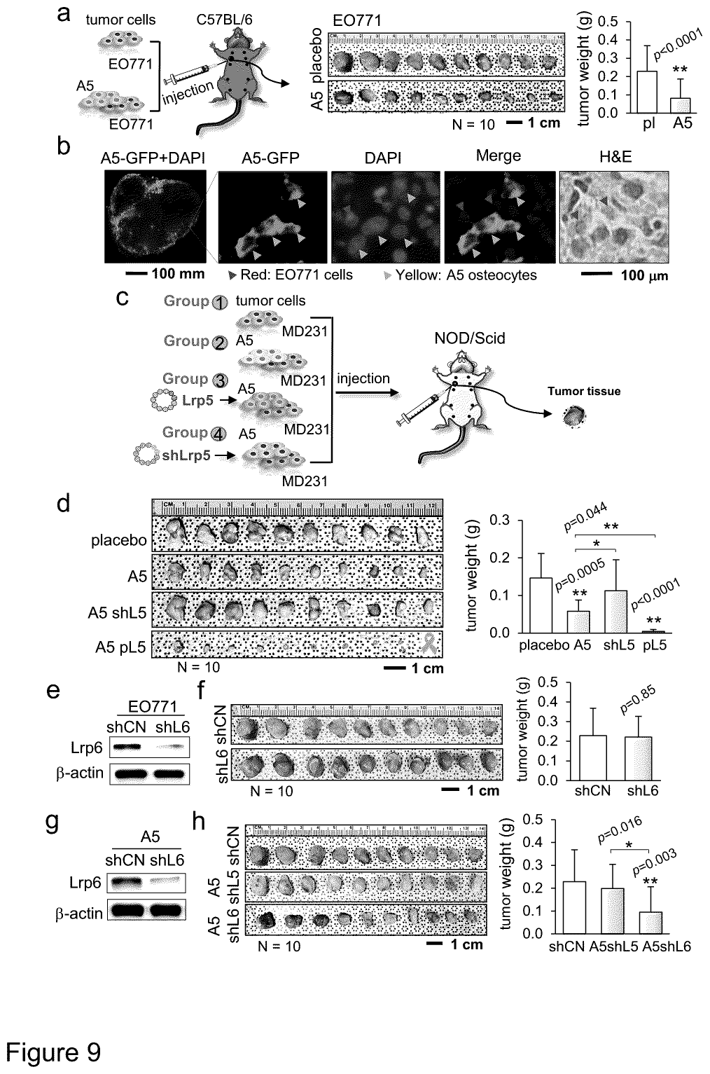

A5 osteocytes reduced mammary tumor growth in vivo. Using a C57BL/6 mouse model, the effect of osteocytes on the mammary tumor were evaluated. EO771 cells were injected into the mammary fat pad with and without osteocyte co-injection. Co-injection of osteocytes reduced the mammary tumors (FIG. 9a). A histological section with GFP-labeled osteocytes showed that osteocytes were mostly located in the proliferating zone, where they were interfused with tumor tissue (FIG. 9b). The same tumor-suppressing effect in NOD/Scid mice with MDA-MB-231 breast cancer cells was observed (FIGS. 9c and 9d). The effect of Lrp6, another co-receptor of Wnt signaling, was different from that of Lrp5. Injection of Lrp6-silenced EO771 cells did not alter mammary tumors (FIGS. 9e and 9f), and Lrp6-silenced osteocyte-derived CM did not suppress the anti-tumor action of osteocytes (FIGS. 9g and 9h). Collectively, Lrp5 but not Lrp6 altered tumor suppression in osteocytes.

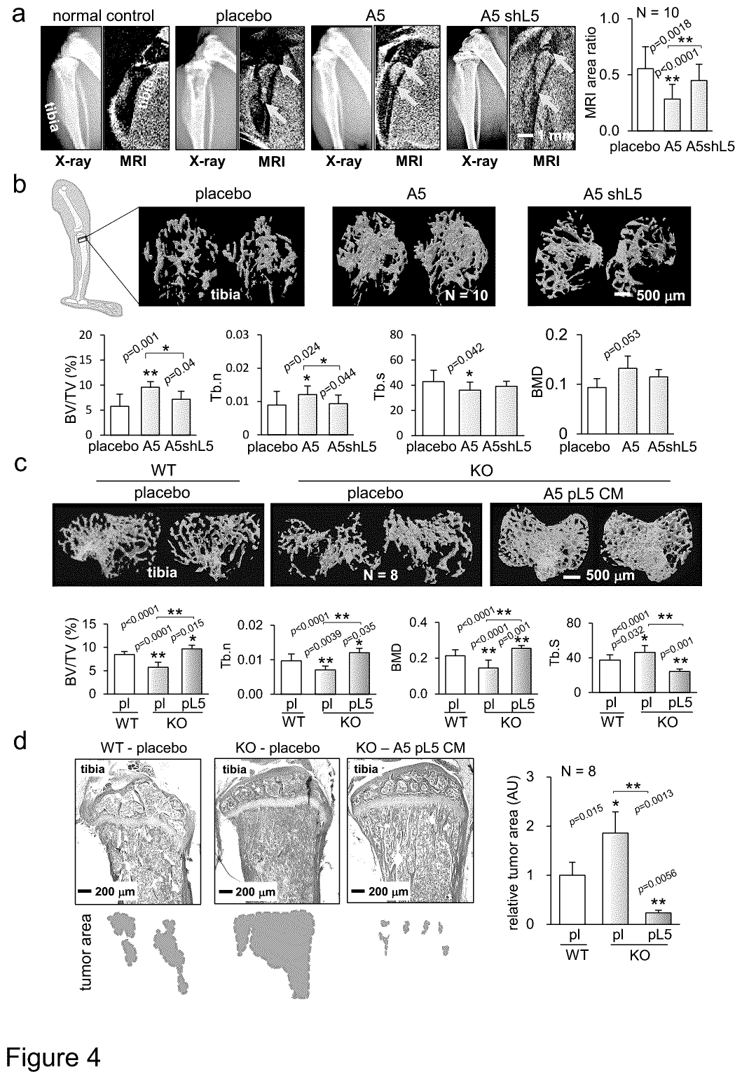

Osteocytes co-injection suppressed tumor-induced osteolysis. So far, it was demonstrated that osteocytes act as a tumor suppressor in the mammary fat pad. The effect on the tumor-invaded bone was examined next. FIG. 4 illustrates the effects of the co-injection of A5 osteocytes to the tibia in C57BL/6 mice. pl=placebo, shL5=Lrp5 shRNA, pL5=Lrp5 plasmids, WT=wildtype, KO=Lrp5 conditional knockout, and CM=conditioned medium. The single and double asterisks indicate p<0.05 and p<0.01, respectively. Representative X-ray and MR images of the tibia in the normal control (healthy group), placebo, A5 co-injected, and Lrp5 shRNA-treated A5 groups are shown in FIG. 4a. The yellow arrows in MR images indicate a tumor-induced lesion in the tibia. MR image-based area ratio of the tumor-induced lesion is shown. Scale bar=1 mm. Representative μCT images of the proximal tibia in the placebo, A5 co-injected, and Lrp5 shRNA-treated A5 groups are shown in FIG. 4b. μCT-based parameters are shown, in which BV/TV=bone volume normalized by total volume, Tb.n=trabecular number, Tb.s=trabecular separation, and BMD=bone mineral density. Scale bar=500 μm. Representative μCT images of the proximal tibia, showing the severe bone loss caused by tumor invasion in Lrp5 KO mice and its rescue by Lrp5-overexpressing osteocyte-derived CM are shown in FIG. 4c. Scale bar=500 H&E stained proximal tibia of Lrp5 KO is shown in FIG. 4d. Lrp5-overexpressing osteocyte-derived CM significantly reduced tumor growth. The green-shaded area showed the tumor-invaded region. Of note, tumor cells typically appeared hyperchromatic with an increased nucleoplasmic ratio. Scale bar=200 μm.

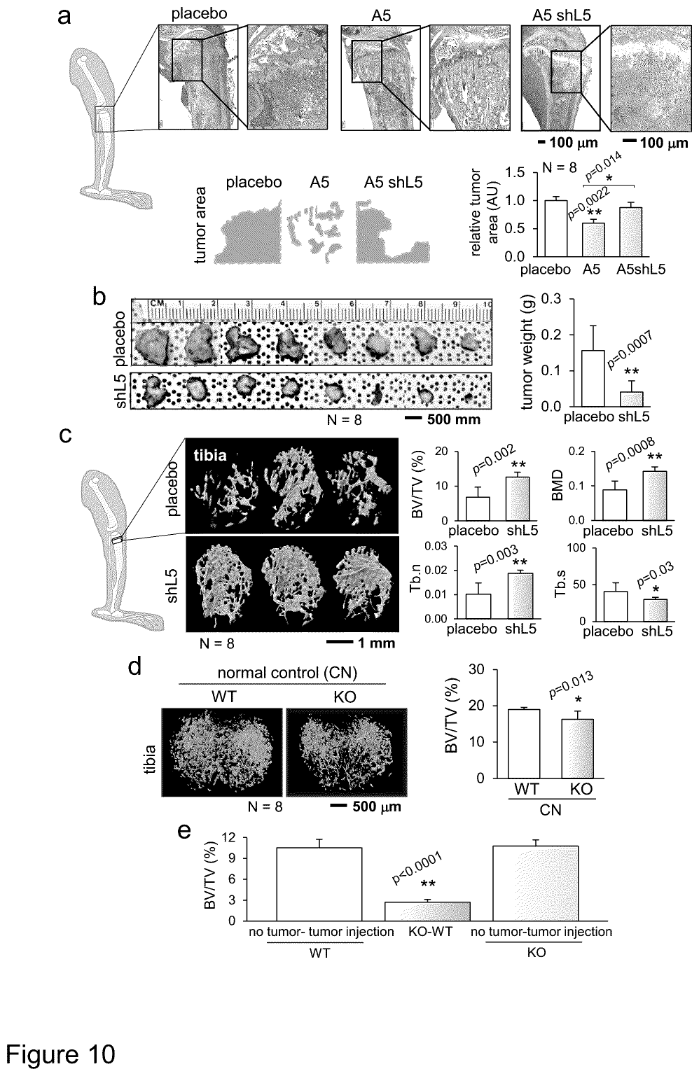

Additionally, further MR imaging of a tibia in C57BL/6 mice showed that the placebo control group (tumor cell alone group) presented clear tumor-linked lesions in the tibia, which were significantly reduced in the A5-injected group (FIG. 10a). Furthermore, μCT imaging revealed that osteocyte co-injection reduced tumor-driven osteolysis (FIG. 10b). In response to the co-injection, the bone volume ratio (BV/TV) and trabecular number (Tb.n) were elevated, and the trabecular separation (Tb.$) was reduced. These changes suggest the ability of osteocytes to protect against cancer-induced osteolysis. No benefits of osteocyte co-injection were observed when osteocytes expressing Lrp5 shRNA were used. H&E-stained sagittal sections revealed that the osteocyte-injected group had reduced tumor growth and bone injury (FIG. 10a). These results indicate that Lrp5-positive osteocytes contributed to the prevention of tumor-induced bone loss. Of note, Lrp5, expressed in tumor cells, has a pro-tumorigenic role as Lrp5-silenced EO771 tumor cells showed a reduction in mammary tumor and tumor-driven bone loss (FIGS. 10b and 10c).

Lrp5 deletion in osteocytes worsened tumor-driven osteolysis in vivo. Having shown Lrp5's anti-tumor capability in osteocytes, the effect of Lrp5 deletion in osteocytes on tumor progression in the tibia was examined using conditional knockout mice. Mice with osteocytic deletion of Lrp5 exhibited significantly lower bone mass than the wildtype littermates. However, local injection of Lrp5-overexpressing osteocyte-derived CM to the proximal tibia markedly protected bone. Besides the tumor-driven reduction in BV/TV, its reduction by the deletion of Lrp5 in osteocytes was also observed (FIG. 10d). However, tumor-induced reduction in BV/TV was more than the reduction caused by the deletion of Lrp5 in osteocytes (FIG. 10e).

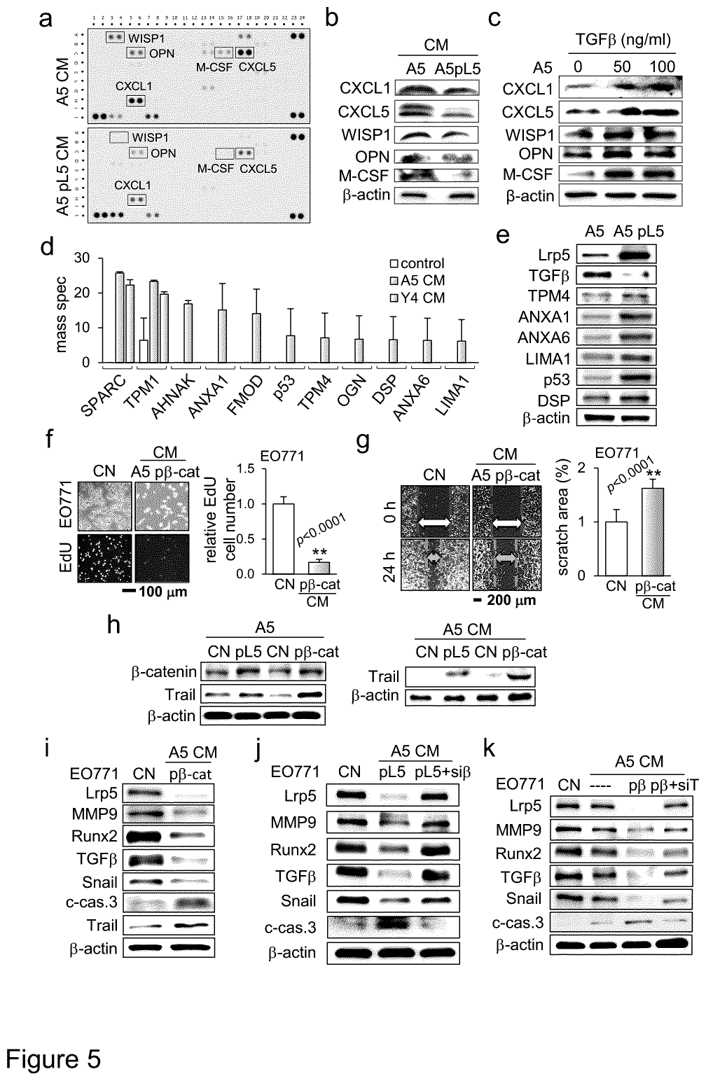

Lrp5 in osteocytes downregulated tumor-promoting genes and upregulated tumor-suppressing genes. To understand the regulatory mechanism of Lrp5's action, antibody array analysis with 111 cytokines was conducted for a pair of osteocyte-derived CMs with and without Lrp5 overexpression. In Lrp5-overexpressing CM, two chemokine ligands (CXCL1 and CXCL5) and three other proteins (WISP1, OPN, and M-CSF) were significantly reduced (FIGS. 5a and 5b;

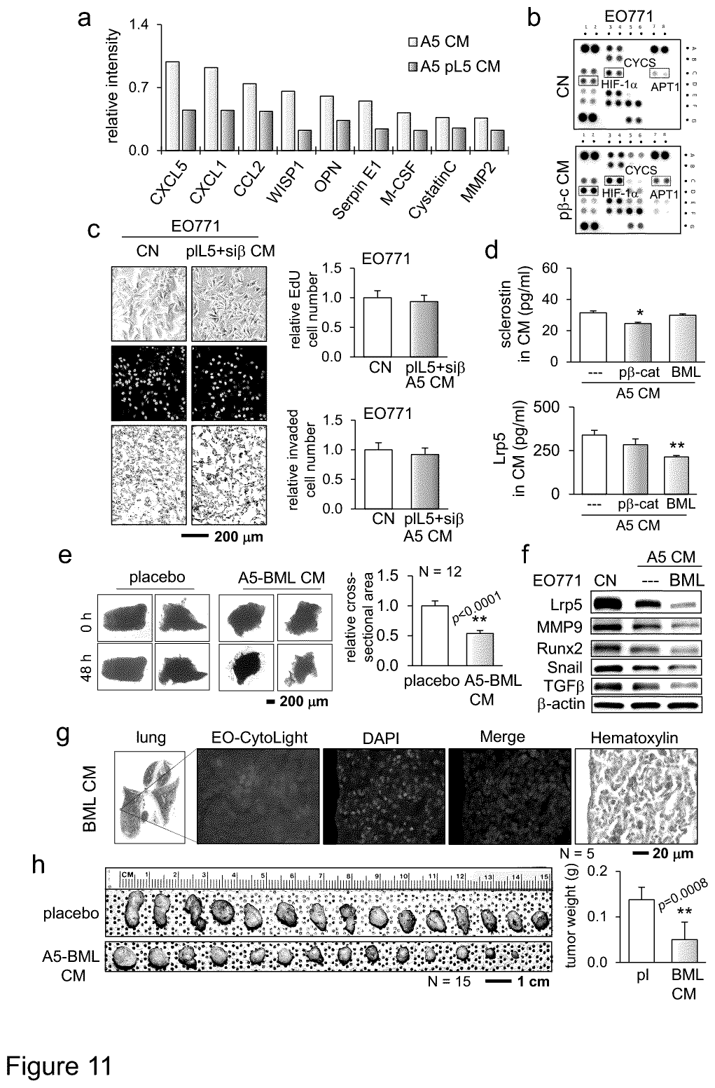

FIG. 11a). These chemokines/cytokines are pro-tumorigenic and pro-metastatic and at least two of the chemokines, CXCL1 and CXCL5, are the targets of the pro-metastatic cytokine TGBβ in osteocytes (FIG. 5c). Besides downregulating tumor-promoting genes, osteocyte-derived CM was enriched with proteins that potentially acted as tumor-suppressors. Mass spectrometry-based analysis predicted that TPM4, ANXA1, ANXA6, LIMA1, p53, and DSP were elevated in A5 osteocyte-derived CM (FIG. 5d). In Lrp5-overexpressing osteocytes, these genes were elevated but TGFβ was reduced (FIG. 5e). The changes in the expression levels of these proteins in osteocyte CM, caused by the overexpression of Lrp5, could contribute to the inhibition of tumor progression.

Overexpression of β-catenin and the Wnt activator, BML284, enhanced the anti-tumor capability of osteocytes. Since Lrp5 is involved in Wnt signaling, the status of β-catenin, which is a downstream mediator of Wnt signaling, was examined. In EO771 cells, β-catenin-overexpressing CM elevated apoptosis-linked genes (CYCS, HIF1α, and APT1) (FIG. 11b). The EdU assay and TRANSWELL®-based invasion assay revealed that β-catenin-overexpressing osteocyte-derived CM reduced the proliferation and migration of EO771 tumor cells (FIGS. 5f and 5g). In osteocytes, the overexpression of Lrp5 increased β-catenin and an apoptosis-inducing factor, Trail (FIG. 5h), while it downregulated tumorigenic proteins such as MMP9, Runx2, TGFβ, and Snail (FIG. 5i). By contrast, the silencing of β-catenin reversed the expression profiles of the overexpression of Lrp5 (FIG. 5j; FIG. 11c). Notably, the inhibitory effect on tumor-promoting proteins by β-catenin-overexpressing CM was suppressed by the silencing of Trail (FIG. 5k).

Levels of Sclerostin and Lrp5 in β-catenin-overexpressing CM and BML284-treated CM were detected, in which BML284 is an activator of Wnt signaling. In both CMs, these proteins were not elevated. Instead, β-catenin overexpressing CM lowered Sclerostin, and BML284-treated CM lowered Lrp5 (FIG. 11d). BML284-treated CM enhanced the anti-tumor capability in an ex vivo tissue assay (FIG. 11e). It also downregulated tumorigenic genes and its administration to C57BL/6 mice reduced the progression of tumors (FIGS. 11f-11h).

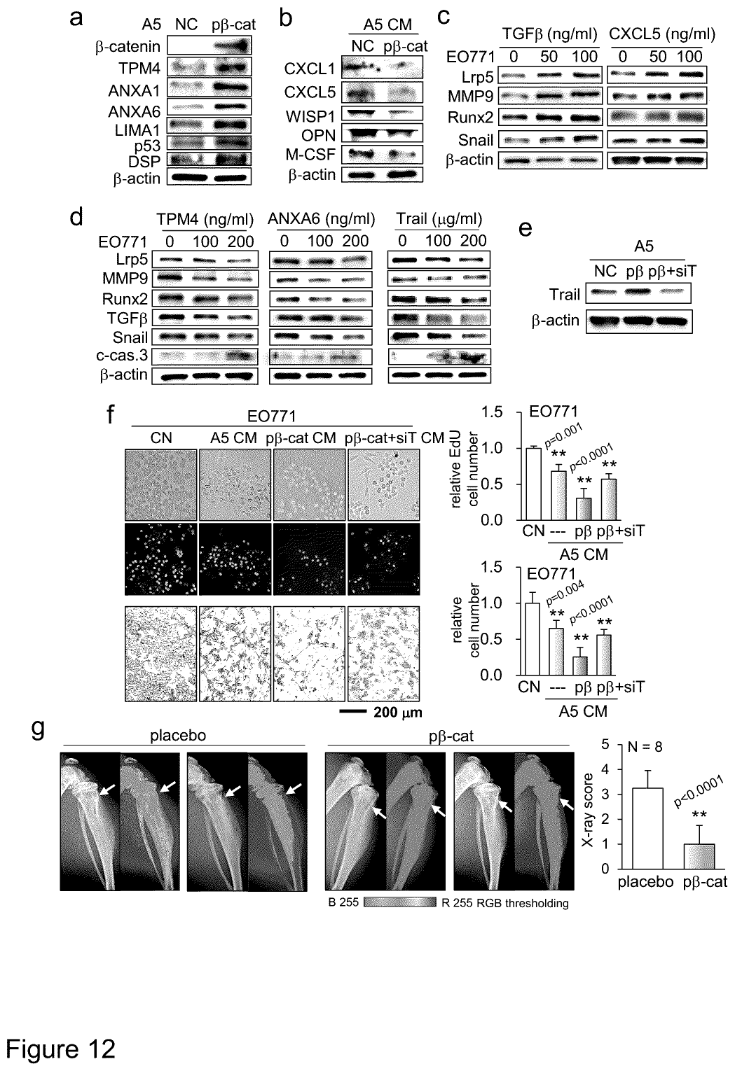

Of note, the tumor-suppressing genes (TPM4, ANXA1, ANXA6, LIMA1, p53, and DSP) were elevated in β-catenin overexpressing osteocytes, and β-catenin overexpressing CM reduced CXCL1, CXCL5, WISP1, OPN, and M-CSF (FIGS. 12a and 12b). Also, the tumor-promoting genes (Lrp5, MMP9, Runx2, and Snail) were increased by TGFβ and CXCL5, while they were reduced by TPM4, ANXA6, and Trail (FIGS. 12c and 12d). Regarding the linkage of β-catenin to Trail, β-catenin-overexpressing CM inhibited the proliferation and invasion of tumor cells, but RNA interference with Trail siRNA blocked the inhibitory effects of β-catenin-overexpressing CM (FIGS. 12e and 12f).

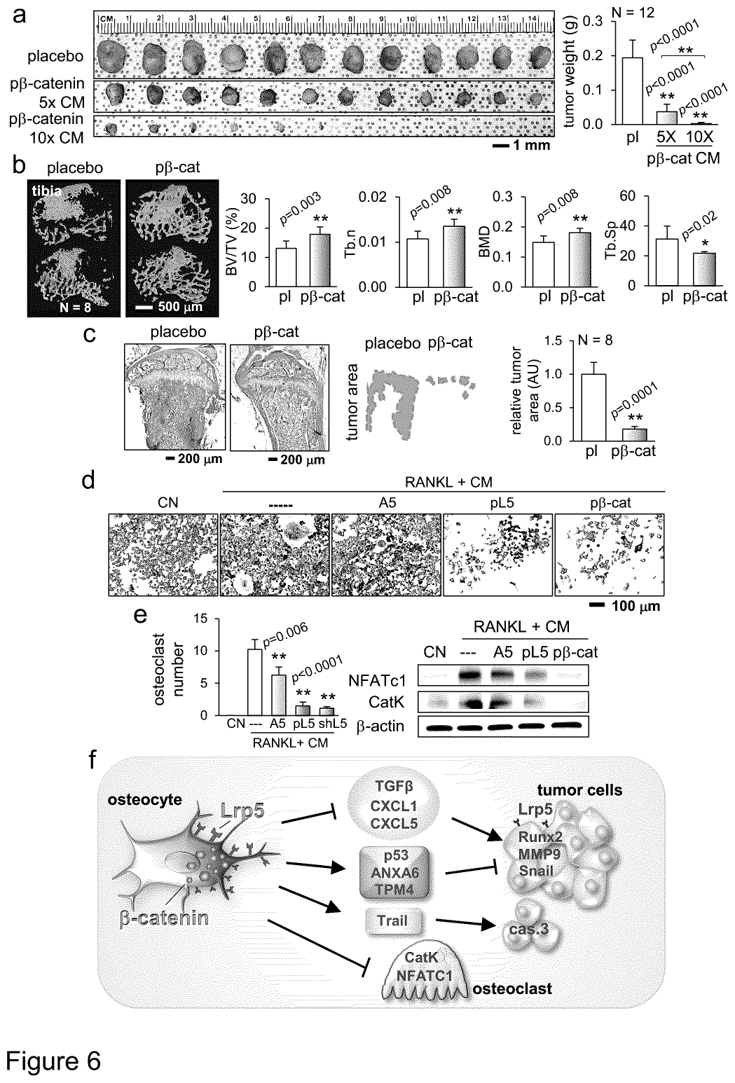

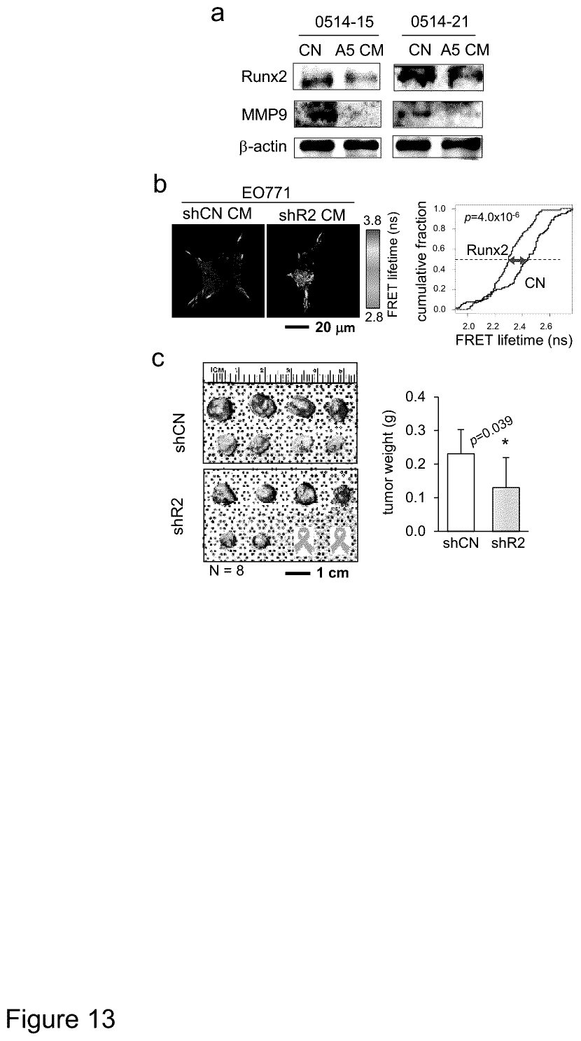

β-catenin-overexpressing CM inhibited tumor progression and osteoclastogenesis. Systemic administration of β-catenin-overexpressing osteocyte-derived CM inhibited the progression of mammary tumors (FIG. 6a) and tumor-driven osteolysis (FIGS. 6b and 6c; FIG. 12g). In addition, the potential involvement of Runx2 in tumor progression was examined. In two sources of human primary breast cancer cells, A5 CM reduced Runx2 and MMP9 (FIG. 13a). FRET analysis revealed that Runx2-silenced EO771 cells decreased FRET lifetime, indicating that the silencing of Runx2 reduced molecular force and cellular migration (FIG. 13b). Furthermore, in a mouse model, the inoculation of Runx2-silenced EO771 cells reduced the tumor weight (FIG. 13c). To examine the effects of osteocyte-derived CM on the development of bone-resorbing osteoclasts, the staining for tartrate-resistant acid phosphatase (TRAP), a marker for osteoclasts, was performed. Notably, Lrp5- and β-catenin-overexpressing CM reduced the number of mature osteoclasts, in which TRAP-positive multinucleated (nuclei>3) cells were counted as mature osteoclasts (FIGS. 6d and 6e). Lrp5- and β-catenin-overexpressing CM downregulated NFATc1, a master transcription factor for osteoclastogenesis, and cathepsin K, a protease for bone resorption (FIG. 6f).

### Example 2

Mesenchymal Stem Cell culture. EO771 mouse mammary tumor cells (CH3 BioSystems, Amherst, NY, USA), 4T1.2 mouse mammary tumor cells (obtained from Dr. R. Anderson at Peter MacCallum Cancer Institute, Australia), and MDA-MB-231 breast cancer cells (ATCC) were cultured in DMEM. RAW264.7 pre-osteoclast cells (ATCC, Manassas, VA, USA) were grown in aMEM. TRAMP-C2ras prostate tumor cells (ATCC) were cultured in DMEM/F-12, and PC-3 human prostate cancer cells (ATCC) were cultured in RPMI-1640 (Gibco, Carlsbad, CA, USA). Murine MSCs derived from the bone marrow of the C57BL/6 strain (Envigo RMS, Inc., Indianapolis, IN, USA) were cultured in MESENCULT™ culture medium (Stem Cell Technology, Cambridge, MA, USA). The culture media was supplemented with 10% fetal bovine serum and antibiotics, and cells were maintained at 37° C. and 5% CO₂. In a three-dimensional spheroid assay, tumor spheroids were formed by culturing cells in the U-bottom low-adhesion 96-well plate (S-Bio, Hudson, NH, USA). To evaluate the effect of MSCs or MSC CM, tumor spheroids were grown with MSC spheroids or MSC-derived CM for 48 h.

MSCs were cultured on a collagen-coated culture dish or in suspension with a magnetic stirrer that was rotated at 100 rpm. CM was prepared from 2×10⁶ cells in 9 ml culture medium with antibiotics and a fraction of fetal bovine serum (“FBS”) consisting of 3 kDa or smaller proteins. After one day of incubation, the medium was condensed 10-fold using a filter to collect 3 kDa or heavier proteins (Thermo-Fisher, Waltham, MA, USA).

In vitro assays. Cellular viability was examined using an MTT assay (Invitrogen, Carlsbad, CA, USA) with the procedure previously described, as well as an EdU assay with a fluorescence-based cell proliferation kit (Thermo-Fisher, Waltham, MA, USA). The recombinant proteins employed included Filamin A, Pkm, Pdia3, Tpm4, Anxa2, Eef1a1, Cts1, Nme2, Dcn, Calr, Aldoa, Calm1, Tpm3, Ppib, Myh9, Ywhae, Hspa5, Hsp90aa1 (MBS962910, MBS8249600, MBS2010131, MBS145304, MBS2009095, MBS2033168, MBS143355, MBS145412, MBS2557309, MBS2009125, MBS8248528, MBS2018713, MBS144696, MBS2009092, MBS717396, MBS143242, MBS806904, MBS142709; MyBioSource, San Diego, CA, USA), Actin Gamma 1, Actn4, Hspa8, Vimentin (H00000071-P01, H00000081-P01, NBP1-30278, NBP2-35139; Novus, Littleton, CO, USA), and Hsp90ab1 (OPCA05157; Aviva system biology, San Diego, CA, USA). A TRANSWELL® chamber assay was conducted to detect invasive cellular motility, and a wound-healing scratch assay was utilized to evaluate 2-dimensional migratory behavior. The overexpression of Akt, Lrp5, β-catenin, Snail, Calr, and Ppib was conducted by transfecting plasmids (#10841, #115907, #31785, #31697, #51161, #36123; Addgene, Cambridge, MA, USA). RNA interference was conducted using siRNA specific to Akt, Lrp5, β-catenin, Snail, and Hsp90ab1 (65496, s69315, s63417, 69332, s67897, Thermo-Fisher) with a negative siRNA (Silencer Select #1, Thermo-Fisher) as a nonspecific control using the procedure previously described.

Western blot analysis and protein array analysis. Western blot analysis was conducted using the procedure previously described. Antibodies against Lrp5, Runx2, Snail, TGFβ, sclerostin, Calr, p-eIF2α, eIF2α (Cell Signaling, Danvers, MA, USA), LIF, Trail (Novus Biologicals, Centennial, CO, USA), MMP9, NFATc1, cathepsin K (Santa Cruz Biotechnology, Dallas, TX, USA), p53, CXCL2, Ppib (Invitrogen, Carlsbad, CA, USA), Hsp90ab1 (Abcam, Cambridge, UK), and β-actin (Sigma, Saint Louis, MO, USA) were used. A proteome profiler mouse XL cytokine array kit (R&D Systems, Minneapolis, MN, USA) was also employed, and the expression of 111 cytokines and chemokines in MSC-derived CM was determined.

Ex vivo breast cancer tissue assay. The usage of human breast cancer tissues was approved by the Indiana University Institutional Review Board. A sample (˜1 g; ER/PR+, HER2+), received from Simon Cancer Center Tissue Procurement Core, was manually fragmented with a scalpel into small pieces (0.5-0.8 mm in length). These pieces were grown in DMEM with 10% fetal bovine serum and antibiotics for a day. MSC-derived CM was then added for two additional days, and the change in the fragment size was determined.

Animal models. The animal procedures were approved by the Indiana University Animal Care and Use Committee and complied with the Guiding Principles in the Care and Use of Animals endorsed by the American Physiological Society. Mice were randomly housed five per cage and provided with mouse chow and water ad libitum. In the mouse model of mammary tumors, 8-week old C57BL/6 female mice and BALB/c female mice (10 mice per group; Envigo RMS, Inc.) received subcutaneous injections of EO771 cells and 4T1.2 cells (3.0×10⁵ cells in 50 μl PBS), respectively, to the mammary fat pad on day 1. In the mouse model of tibial osteolysis, C57BL/6 female mice and BALB/c female mice (10 mice per group) received an injection of EO771 cells and 4T1.2 cells (3.0×10⁵ cells in 20 μl PBS), respectively, to the right tibia as an intra-tibial injection,

For examining the anti-tumor efficacy of MSCs, primary mouse MSCs (1.5×10⁵ cells in 50 μl PBS), transfected with or without Lrp5 plasmids, were co-injected (3.0×10⁵ cells) with EO771 cells to the mammary fat pad. For examining the efficacy of MSC-derived CM, CM was condensed by a filter with a cutoff molecular weight of 3 kDa and the 10-fold condensed CM (50 μl re-suspended in PBS) was intravenously injected from the tail vein. The animals were sacrificed on day 14 and mammary tumors and tibiae were harvested.

μCT imaging and histology. The tibiae were blindly labeled and analyzed using μCT imaging and histology. Micro-computed tomography was performed using SKYSCAN™ 1172 (Bruker-MicroCT, Kontich, Belgium). Using manufacturer-provided software, scans were performed at pixel size 8.99 μm and the images were reconstructed (nRecon v1.6.9.18) and analyzed (CTan v1.13). Using μCT images, trabecular bone parameters such as bone volume ratio (BV/TV), bone mineral density (BMD), trabecular number (Tb.N), and trabecular separation (Tb.Sp.) were determined. In histology, H&E staining was conducted as described previously. Normal bone cells appeared in a regular shape with round and deeply stained nuclei, while tumor cells were in a distorted shape with irregularly stained nuclei. X-ray imaging was also conducted with a FAXITRON™ radiographic system (Faxitron X-ray Co.).

Mass spectrometry-based proteomics analysis. Proteins in CM were analyzed in the Dionex ULTIMATE™ 3000 RSLC nano system combined with the Q-exactive high-field hybrid quadrupole orbitrap mass spectrometer (Thermo Fisher Scientific). Proteins were first digested on-beads using trypsin/LysC as described previously except digestion was performed in 50 mM ammonium bicarbonate buffer instead of urea. Digested peptides were then desalted using mini spin C18 spin columns (The Nest Group, Southborough, MA, USA) and separated using a trap and 50-cm analytical columns. Raw data were processed using MAXQUANT™ (v1.6.3.3) against the Uniprot mouse protein database at a 1% false discovery rate allowing up to 2 missed cleavages. MS/MS counts were used for relative protein quantitation. Proteins identified with at least 1 unique peptide and 2 MS/MS counts were considered for the final analysis.

Statistical analysis. The number of animals per group was determined based on power analysis to achieve a power of 80% with p<0.05. For cell-based experiments, three or four independent experiments were conducted and data were expressed as mean±S.D. Statistical significance was evaluated using a one-way analysis of variance (ANOVA). Post hoc statistical comparisons with control groups were performed using Bonferroni correction with statistical significance at p<0.05. The single and double asterisks in the figures indicate p<0.05 and p<0.01, respectively.

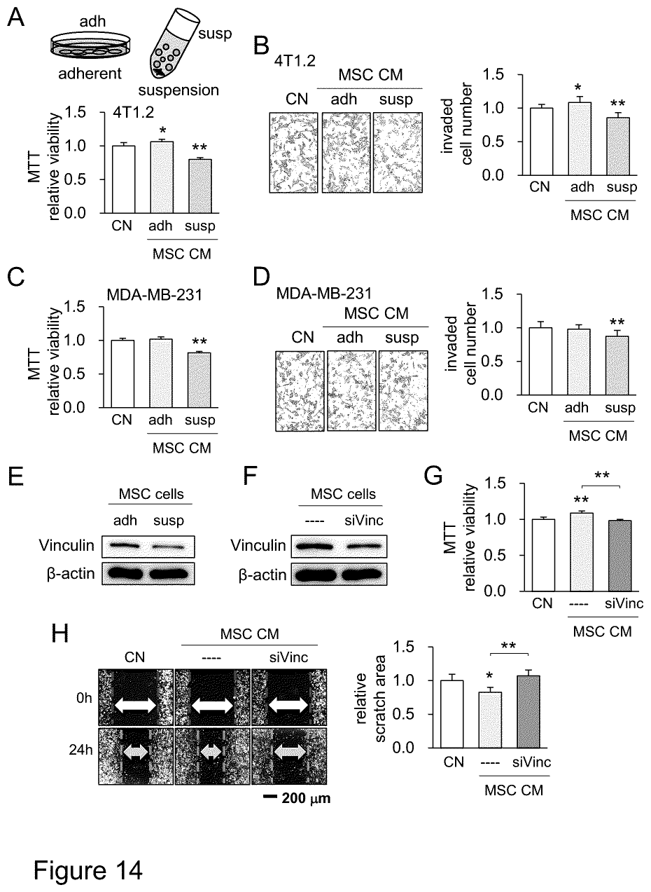

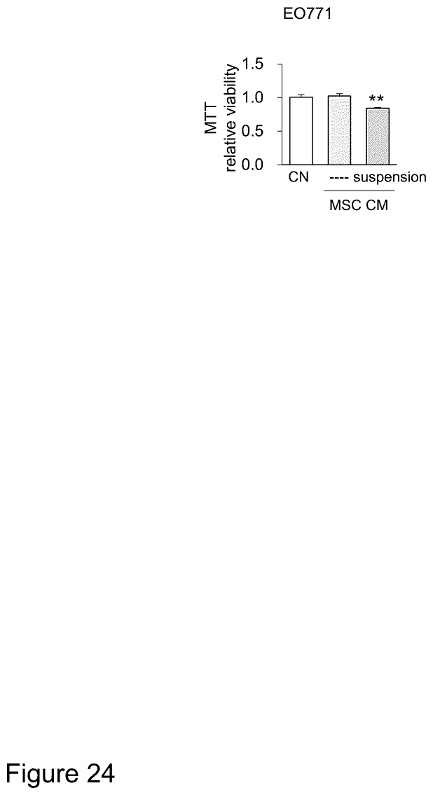

Tumor-suppressing effects of MSCs in suspension culture. When MSCs were cultured on the adhesive surface, their CM did not present any obvious tumor-suppressing action (FIG. 14A). However, when they were grown in suspension culture, the CM exhibited tumor-suppressing capability by reducing MTT-based cell viability of 4T1.2 and EO771 mammary tumor cells (FIG. 14A, FIG. 24) and TRANSWELL®-based invasion of 4T1.2 cells (FIG. 14B), as well as MDA-MB-231 breast cancer cells (FIGS. 14C and 14D). In MSCs cultured in suspension, Western blot analysis revealed that the expression of vinculin was reduced (FIG. 14E). To evaluate the role of vinculin, vinculin in MSCs was silenced using RNA interference (FIG. 14F). Interestingly, vinculin-silenced MSCs grown on the adhesive surface gained the tumor-suppressing capability, as evidenced by reductions of MTT-based cell viability and scratch-based migration of 4T1.2 mammary tumor cells (FIGS. 14G and 14H). These results indicated the possibility of inducing tumor-suppressing capability in MSCs by altering the expression level of a single gene such as vinculin.

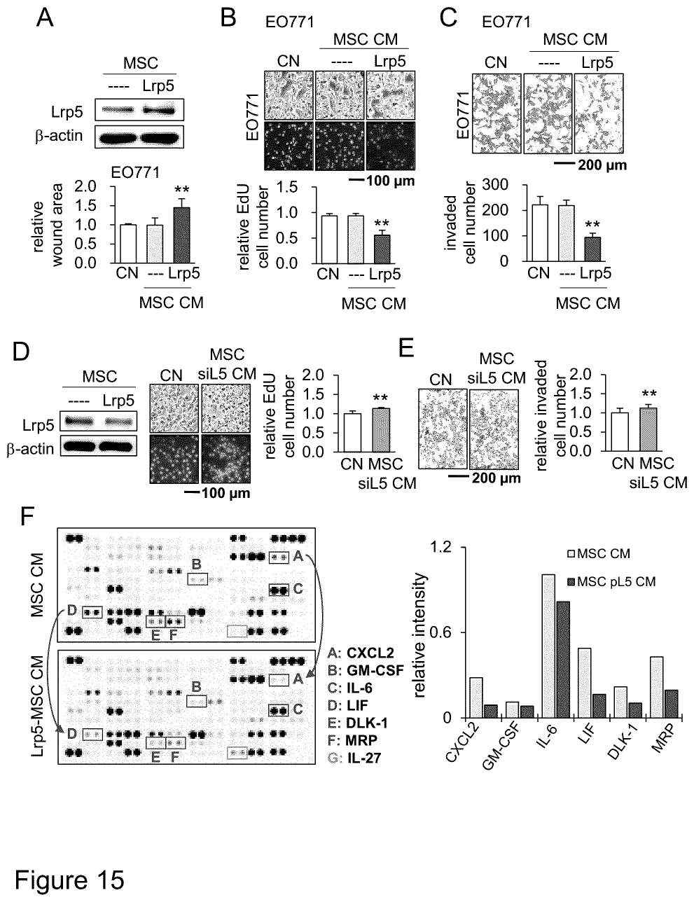

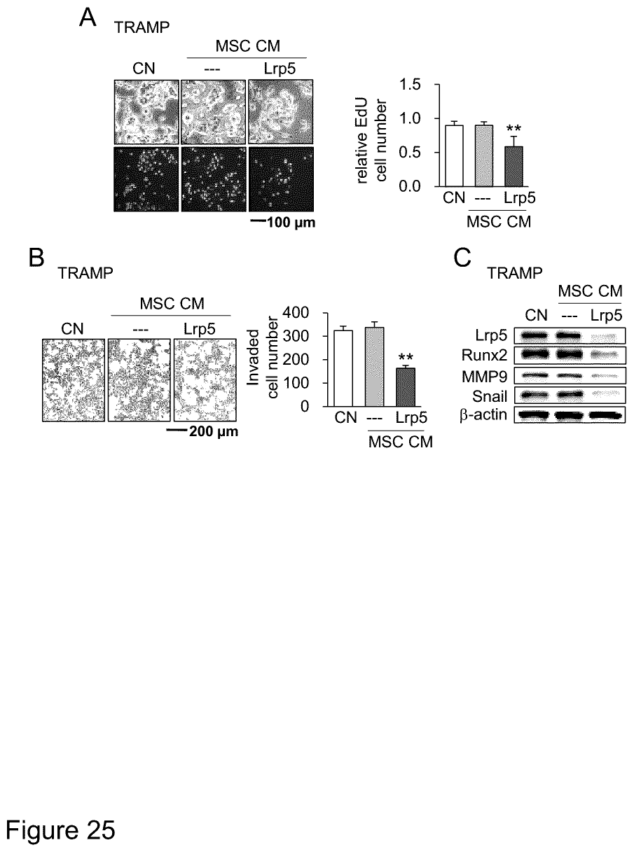

Tumor-suppressing effects of Lrp5-overexpressing MSC CM. The role of Lrp5-mediated Wnt signaling in loading-driven bone formation has been evaluated. Herein, whether Lrp5 would regulate the tumor-suppressing capability of MSCs was examined. Notably, Lrp5-overexpressing MSC CM reduced the scratch-based migration, EdU-based proliferation, and TRANSWELL®-based invasion of EO771 cells (FIGS. 15A-15C). In response to Lrp5-overexpressing MSC CM, TRAMP prostate tumor cells also reduced EdU-based proliferation, and TRANSWELL®-based invasion, and downregulated tumorigenic genes such as Lrp5, Runx2, MMP9, and Snail (FIG. 25). By contrast, Lrp5-silenced MSC CM yielded the opposite outcome (FIGS. 15D and 15E). In cytokine and chemokine array analyses, the overexpression of Lrp5 reduced the levels of several tumor-promoting factors in CM, including CXCL2, GM-CSF, IL6, LIF, DLK1, and MRP, with an increase in IL27, a tumor-suppressing cytokine (FIG. 15F).

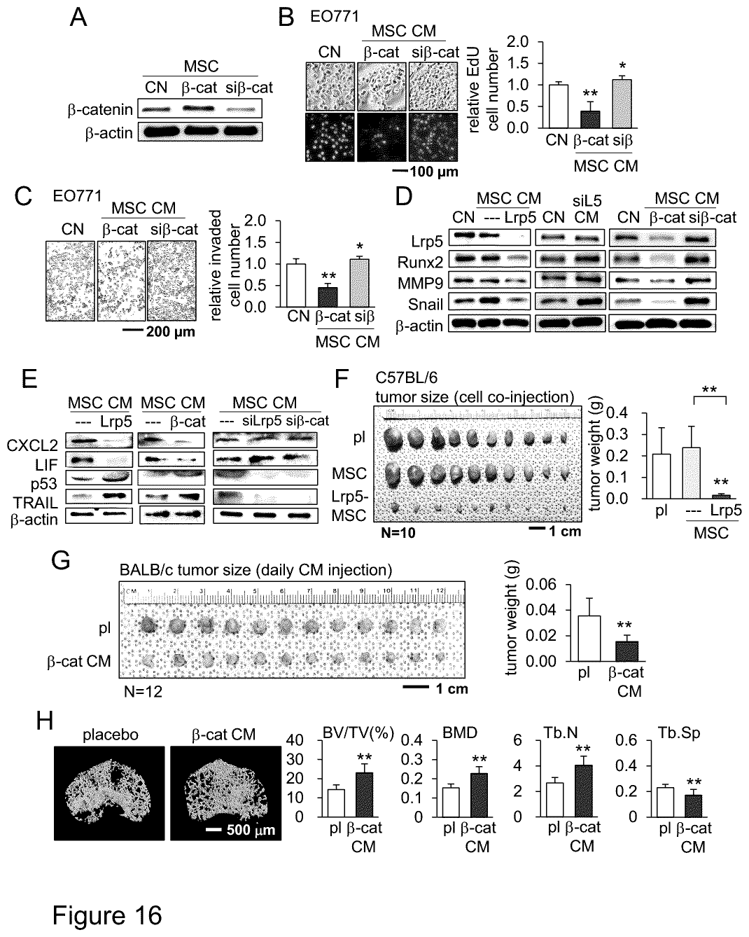

Tumor-suppressing effects of β-catenin-overexpressing MSC CMs. The effects of β-catenin overexpression in MSCs were next examined. Similar to the results of overexpressing Lrp5, β-catenin-overexpression in MSCs led to the production of CM that inhibited the proliferation and invasion of EO771 cells. On the other hand, silencing β-catenin resulted in a slight increase in EO771 cell proliferation and invasion (FIGS. 16A-16C). Western blot analyses revealed the downregulation of oncogenic genes such as Lrp5, Runx2, MMP9, and Snail in EO771 cells cultured in CM produced by MSCs with Lrp5- or β-catenin-overexpression. The expressions of these oncogenic genes in EO771 cells were upregulated when the cells were cultured in CM produced by MSCs with Lrp5- or β-catenin silencing (FIG. 16D). The expression of tumor promoters CXCL2 and LIF, as well as tumor suppressors p53 and Trail in EO771 cells, was next evaluated. The overexpression of Lrp5 or β-catenin in MSCs downregulated CXCL2 and LIF and upregulated p53 and Trail in their CMs, while their silencing reversely regulated these proteins (FIG. 16E). In the C57BL/6 mouse model, the co-injection of Lrp5-overexpressing MSCs to the mammary fat pad significantly reduced the weight of EO771 cell-driven tumors (FIG. 16F). The tumor-suppressing effect was also observed by the daily intravenous injection of β-catenin-overexpressing MSC CM in 4T1.2 tumor cell-inoculated BALB/c mice (FIG. 16G). Furthermore, μCT imaging revealed that tumor-driven osteolysis in the proximal tibia of BALB/c mice was significantly reduced by the daily injection of β-catenin-overexpressing MSC CM (FIG. 16H).

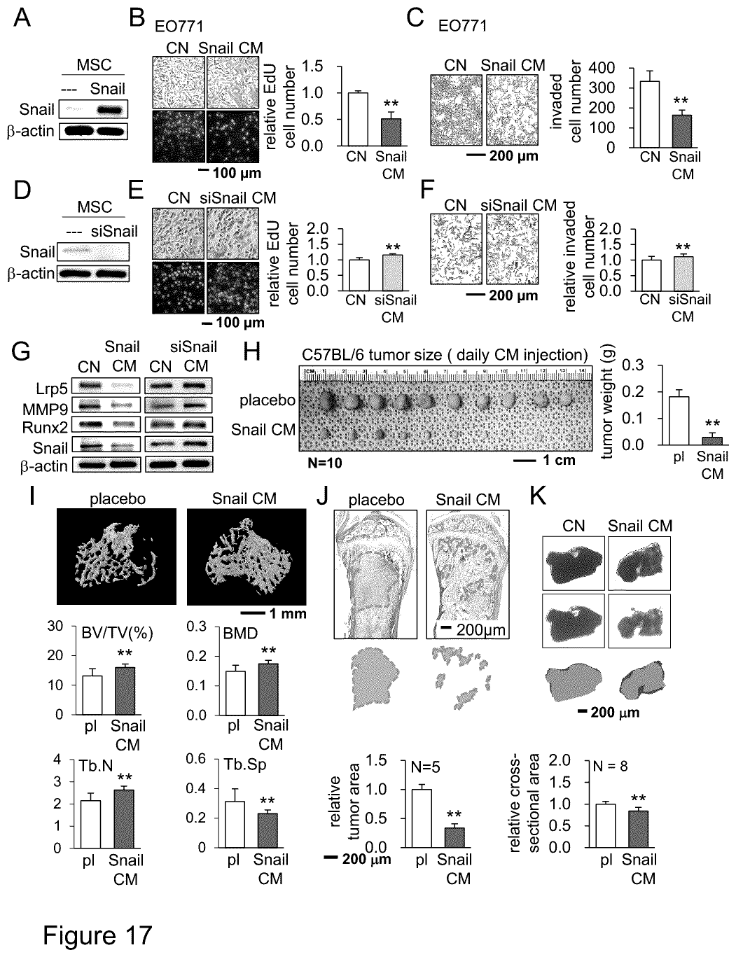

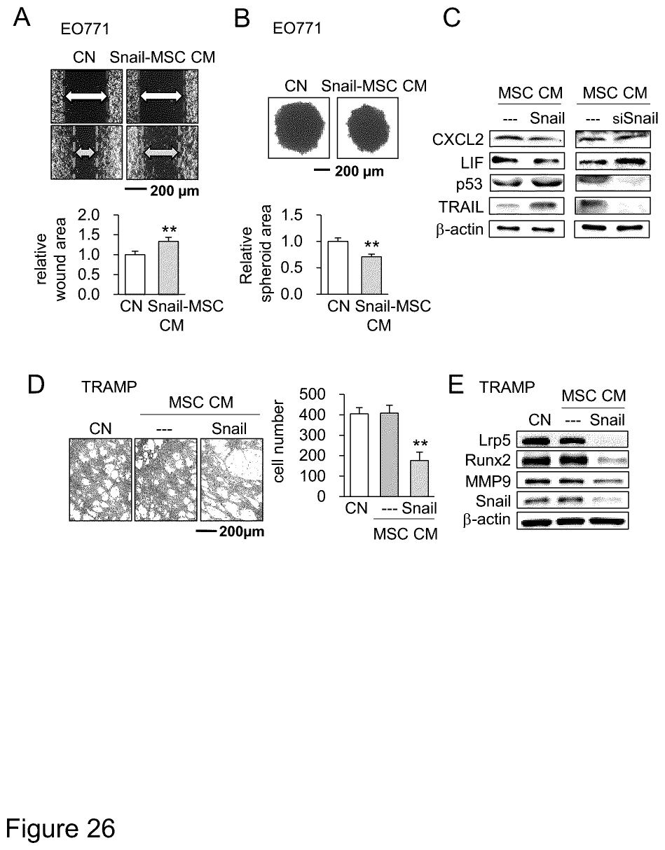

Tumor-suppressing capability by the overexpression of Snail in MSCs. Strong evidence of the anti-tumor capability of MSC CMs by the overexpression of Lrp5 or β-catenin has been presented. Overexpressed Snail, which was activated by Wnt signaling and is a critical mesenchymal marker in EMT. Strikingly, the overexpression of Snail also supported our unconventional hypothesis. Snail-overexpressing MSC CM reduced EdU-based proliferation (FIG. 4A&B), TRANSWELL®-based invasion, and scratch-based migration (FIG. 17C, FIG. 26A), as well as the growth of EO771 tumor spheroids (FIG. 26B). By contrast, Snail-silenced MSC CM reversed the responses (FIGS. 17D-17F). The selected tumor-promoting genes (Lrp5, MMP9, Runx2, and Snail) in EO771 cells were decreased by treating with Snail-overexpressing MSC CMs and the effect was reversed by silencing Snail (FIG. 17G). In CM produced by Snail-overexpressing MSCs, CXCL2 and LIF were significantly reduced, whereas p53 and Trail were upregulated. The expression pattern was converted by silencing Snail in MSCs (FIG. 26C). In prostate cancer cells (TRAMP), the overexpression of Snail also reduced the TRANSWELL®-based invasion and downregulated the tumor-promoting genes (FIGS. 26D and 26E).

The in vivo result of C57BL/6 mice with EO771 cells clearly showed that the daily intravenous injection of Snail-overexpressing MSC CM from the tail vein inhibited tumor progression in the mammary fat pad (FIG. 17H) and the tibia (FIGS. 171 and 17J). In the proximal tibia, Snail-overexpressing MSC CM elevated the bone volume ratio (BV/TV) and bone mineral content (BMD). It also strengthened the porous trabecular bone by increasing the trabecular number (Tb.N) and decreasing the trabecular separation (Tb. Sp). Furthermore, in an ex vivo tissue assay with freshly isolated human breast cancer tissues, the shrinking of cancer tissue fragments via Snail-overexpressing MSC-CM treatment (FIG. 17K) was demonstrated.

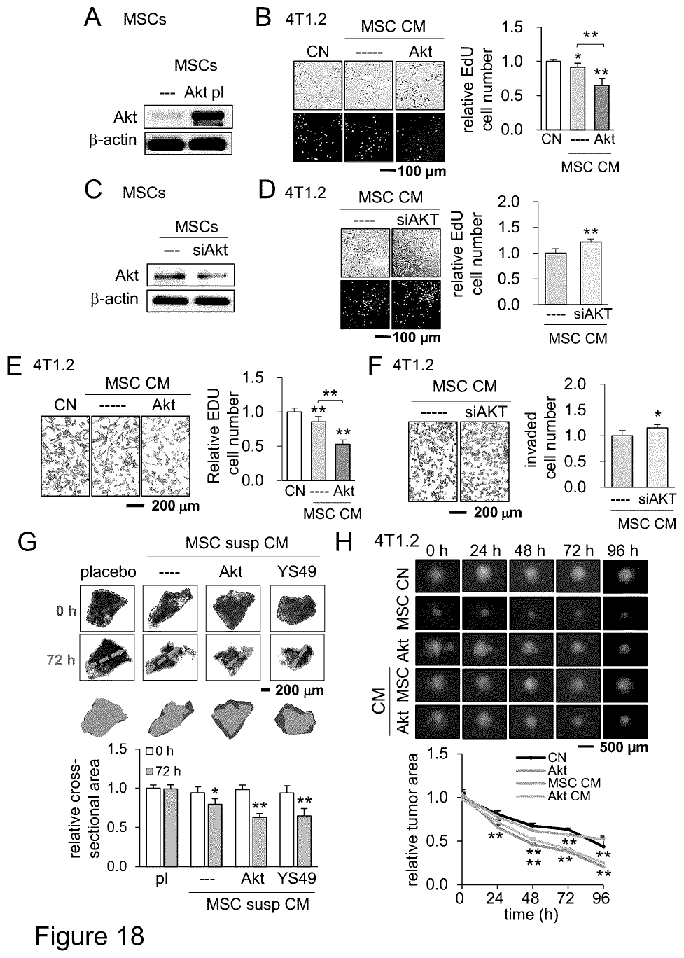

Tumor-suppressing capability by the overexpression of Akt in MSCs. The effect of activating PI3K signaling by overexpressing Akt in MSCs was evaluated. The result presented the same trend as the overexpression of Lrp5, β-catenin, or Snail. MSC CM with the overexpression of Akt reduced the EdU-based proliferation of 4T1.2 cells (FIGS. 18A and 18B), while Akt-silenced MSC CM elevated it (FIGS. 18C and 18D). Similarly, Akt-overexpressing MSC CM inhibited the TRANSWELL®-based invasion of 4T1.2 cells, while Akt-silenced MSC CM promoted it (FIGS. 18E and 18F).

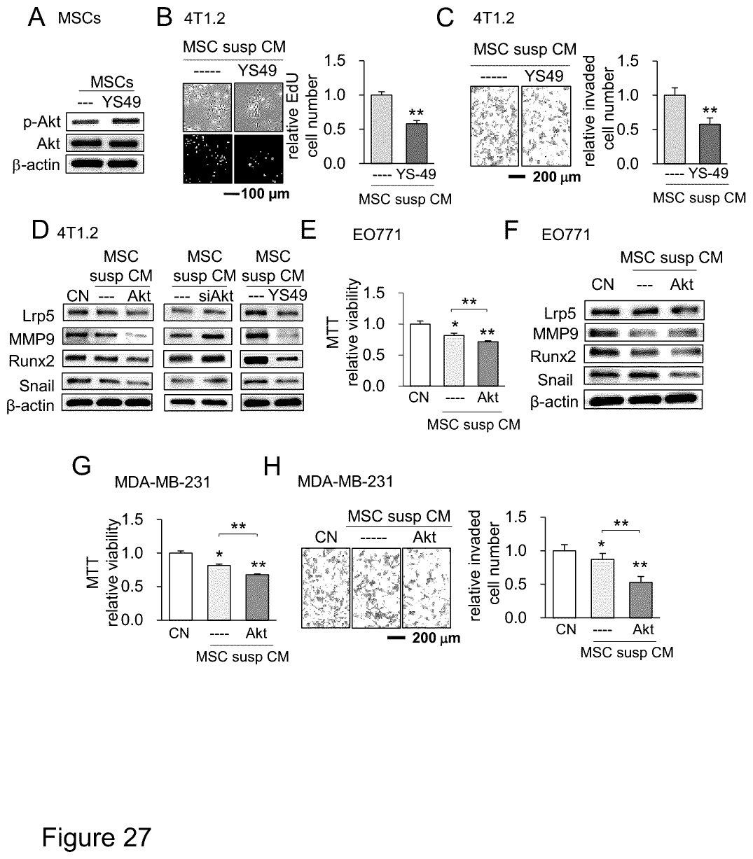

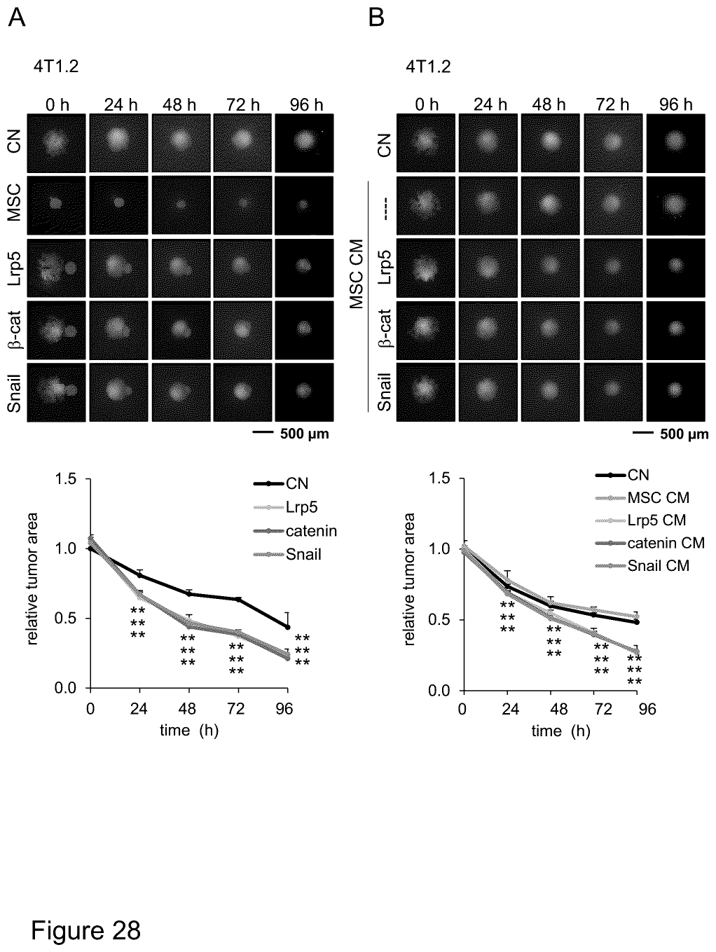

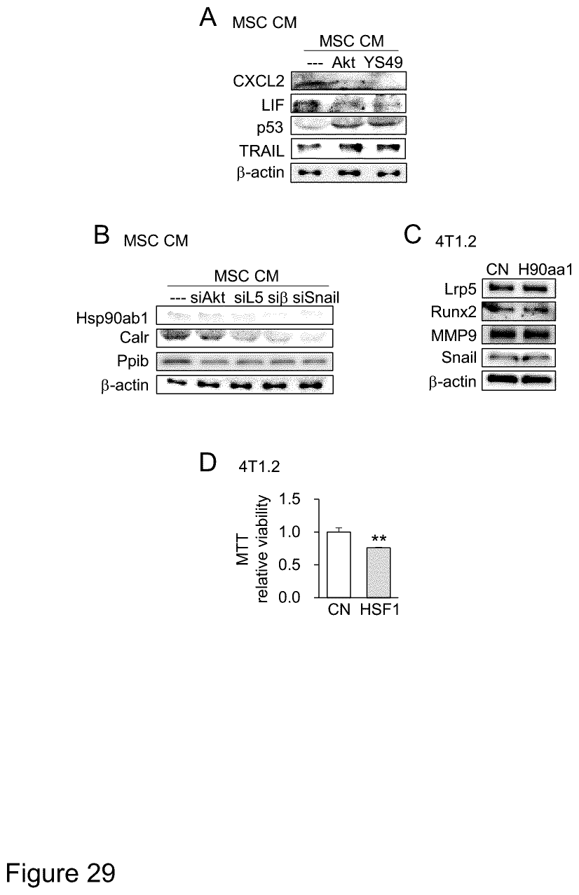

In addition to Akt overexpression, whether activating PI3K signaling via YS49, a pharmacological agent, would achieve the same anti-tumor effect was tested. The administration of YS49 to MSCs elevated the phosphorylated Akt in MSCs (FIG. 27A), and Y549-treated MSC CM reduced EdU-based proliferation and TRANSWELL®-based invasion of 4T1.2 cells (FIGS. 27B and 27C). Furthermore, both Akt-overexpressing and Y549-treated MSC CMs inhibited the ex vivo growth of tumor fragments that were derived from freshly isolated ER-positive human breast cancer tissues (FIG. 18G). Akt-overexpressing and Y549-treated MSC CMs downregulated the oncogenic genes such as Lrp5, MMP9, Runx2, TGFβ, and Snail in 4T1.2 cells and EO771 cells and reduced the MTT-based cell viability, while Akt-silenced MSC CM elevated those genes (FIGS. 27D and 27F). The tumor-suppressing effect of Akt overexpression was observed not only in 4T1.2 cells but also in EO771 cells and MDA-MB-231 breast cancer cells (FIGS. 27G and 27H). Lastly, a spheroid competition assay was conducted and evaluated the effect of MSC-tumor interactions. The result revealed that Akt-, Lrp5-, β-catenin-, and Snail-overexpressing MSCs and their CMs significantly inhibited the growth of 4T1.2 tumor spheroids (FIG. 18H, FIG. 28). Also, the levels of CXCL2 and LIF were lowered and those of p53 and Trail were elevated in Akt-overexpressing and Y549-treated MSC CMs (FIG. 29A).

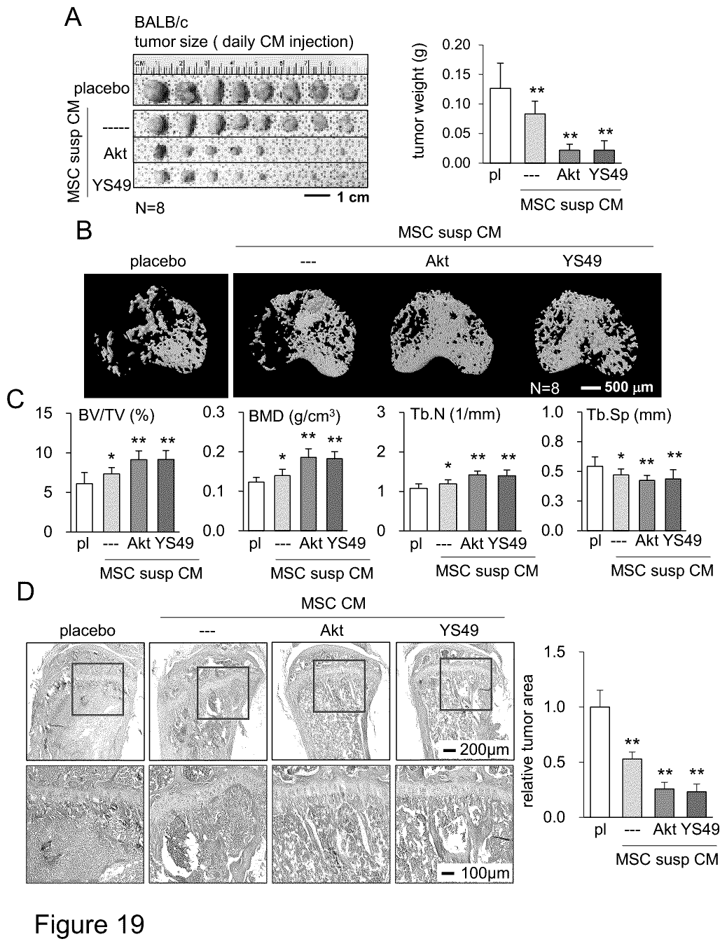

Suppression of mammary tumors and bone degradation by Akt overexpression. Consistent with the in vitro and ex vivo results, the daily intravenous injection of Akt-overexpressing and Y549-treated MSC CMs reduced the weight of mammary tumors in 4T1.2 tumor cell-inoculated BALB/c mice (FIG. 19A). Furthermore, bone loss in tumor-invaded tibia was suppressed with CMs produced by Akt-overexpressing and YS49 treated MSCs (FIG. 19B). Furthermore, the engineered MSC CMs led to an increase in the bone volume ratio, bone mineral density, and trabecular number, with a decrease in trabecular separation (FIG. 19C). H&E-stained histological sections revealed that the tumor-invaded area was significantly reduced by the administration of Akt-overexpressing and Y549-treated MSC CMs (FIG. 19D). Collectively, these results support the protection of bone by the daily intravenous administration of MSC CMs.

Hsp90ab1, calreticulin, and peptidylprolyl isomerase B as tumor-suppressing proteins. Mass spectrometry-based proteomics analysis was conducted to identify the proteins in the MSC CM that were critical for the tumor-suppressing action. Focusing on the PI3K signaling pathway, 4 CMs (medium control without MSCs, MSC CM control, Akt-overexpressing CM, and Y549-treated CM) were employed and a total of 885 proteins were identified. There were 104 proteins identified in the Akt-overexpressing MSC CM, and 75 proteins were expressed at a higher level in both Akt-overexpressing and Y549-treated CM than the control CM (Table 1). Table 1 below contains a list of 75 proteins that were expressed higher in Akt-overexpressing and Y549-treated CMs than the control CM in mass spectrometry-based proteonomics analysis.

TABLE 1

| | Mol. Wt. | Expression Level (MS/MS Count) | | |
|---|---|---|---|---|
| Gene Name | (kDa) | CN | Akt | YS49 |
| Hspa5 | 72.4 | 0 | 25 | 45 |
| Actg1 | 41.8 | 20 | 40 | 221 |
| Flna | 280.5 | 0 | 18 | 83 |
| Vim | 53.7 | 3 | 20 | 51 |
| Hsp90ab1 | 83.3 | 15 | 27 | 101 |
| Hspa8 | 70.9 | 8 | 19 | 160 |
| Actn4 | 105.0 | 4 | 14 | 27 |
| Pkm | 57.8 | 8 | 17 | 133 |
| P4hb | 56.6 | 0 | 9 | 7 |
| Pdia3 | 56.7 | 0 | 9 | 6 |
| Tpm4 | 28.5 | 0 | 9 | 4 |
| Anxa2 | 38.6 | 3 | 11 | 72 |
| Msn | 67.7 | 0 | 8 | 9 |
| Eef1a1 | 50.1 | 4 | 11 | 100 |
| Ctsl | 37.6 | 0 | 7 | 14 |
| Gm20390; Nme2; Nme1 | 30.2 | 4 | 11 | 9 |
| Dcn | 39.8 | 0 | 7 | 5 |
| Calr | 42.2 | 14 | 20 | 30 |
| Aldoa | 39.4 | 0 | 6 | 20 |
| Calm1 | 16.8 | 3 | 9 | 20 |
| Tpm3; Tpm3-rs7 | 29.0 | 2 | 8 | 12 |
| Ppib | 23.7 | 0 | 6 | 9 |
| Tuba1b; Tuba1c; Tuba1a | 50.2 | 2 | 7 | 166 |
| Myh9 | 226.4 | 0 | 5 | 87 |
| Ywhae | 29.2 | 14 | 19 | 38 |
| Hsp90aa1 | 84.8 | 0 | 5 | 32 |
| Vcp | 89.3 | 7 | 12 | 27 |
| Actn1 | 103.1 | 0 | 5 | 13 |
| Psma7; Psma8 | 27.9 | 0 | 5 | 13 |
| Actb; Actg1 | 41.8 | 0 | 5 | 2 |
| Tubb5 | 49.7 | 3 | 7 | 116 |
| Got2 | 47.4 | 6 | 10 | 20 |
| Mdh2 | 35.6 | 0 | 4 | 13 |
| Calu | 37.1 | 0 | 4 | 13 |
| Prdx1 | 22.2 | 0 | 4 | 11 |
| Ncl | 76.9 | 2 | 6 | 11 |
| Hspa4 | 94.2 | 0 | 4 | 9 |
| C3 | 186.5 | 11 | 15 | 18 |
| Arhgdia | 23.4 | 0 | 4 | 4 |
| EG433182; Eno1 | 47.1 | 8 | 11 | 116 |
| Atp5b | 56.3 | 0 | 3 | 72 |
| Plec | 506.5 | 0 | 3 | 71 |
| Eif4a1 | 46.0 | 0 | 3 | 49 |
| Gm1821; Ubc; Gm8797; | 17.2 | 3 | 6 | 28 |
| Uba52; Rps27a; Kxd1; Ubb | | | | |
| Lmna | 74.2 | 0 | 3 | 22 |
| Eef1g | 50.1 | 0 | 3 | 16 |
| Ctsb | 37.3 | 9 | 12 | 17 |
| Got1 | 46.2 | 2 | 5 | 10 |
| GAPDH; Gapdh; Gm3839 | 35.8 | 0 | 3 | 8 |
| Actg1 | 43.6 | 0 | 3 | 4 |
| Eef2 | 95.3 | 0 | 2 | 48 |
| Flnb | 277.8 | 0 | 2 | 42 |
| Rps3 | 26.7 | 0 | 2 | 35 |
| Pgk1 | 44.6 | 2 | 4 | 36 |
| Hnmpa2b1 | 37.4 | 0 | 2 | 25 |
| Hsp90b1 | 92.5 | 0 | 2 | 13 |
| Mif | 12.5 | 2 | 4 | 14 |
| Pgam1 | 38.8 | 2 | 4 | 11 |
| Ppia | 18.0 | 0 | 2 | 8 |
| Hmgb1 | 19.8 | 0 | 2 | 6 |
| Prdx2 | 21.8 | 0 | 2 | 6 |
| Pdia4 | 72.4 | 0 | 2 | 6 |
| Psmb1 | 15.6 | 0 | 2 | 5 |
| Sh3d21 | 26.8 | 16 | 18 | 20 |
| Ybx1 | 35.7 | 0 | 2 | 4 |
| Actb | 41.8 | 0 | 2 | 4 |
| Pdia6 | 48.7 | 0 | 2 | 4 |
| Akr1a1 | 36.6 | 0 | 2 | 4 |
| Marcks | 29.7 | 2 | 4 | 5 |
| Psma1 | 29.5 | 0 | 2 | 3 |
| Tmsb4x | 5.1 | 0 | 2 | 3 |
| Ahnak | 604.3 | 0 | 2 | 2 |
| Ywhag | 28.3 | 2 | 4 | 3 |
| Set; BC085271 | 17.6 | 2 | 3 | 6 |
| Aga | 37.0 | 2 | 3 | 6 |

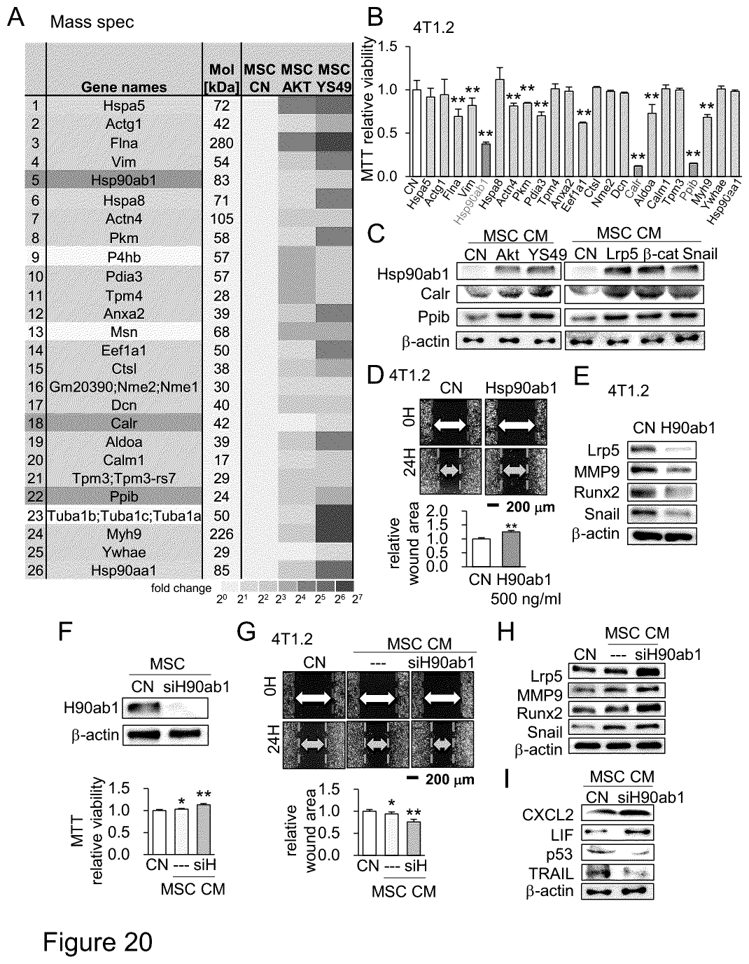

As potential tumor suppressors, 26 top candidates are listed, and based on the availability of recombinant proteins, the effects of 23 proteins on the MTT-based viability of 4T1.2 tumor cells were evaluated (FIG. 20A). Among the 23 proteins, 11 proteins induced a statistically significant decrease in MMT-based viability (FIG. 20B). Four heat shock proteins (Hspa5, Hsp90ab1, Hspa8, and Hsp90aa1) were included in the top candidate list and Hsp90ab1 was predicted to be one of the most influential tumor suppressors, as well as calreticulin (Calr) and peptidylprolyl isomerase B (Ppib). The role of these three proteins that were upregulated in MSC CM by the overexpression of Akt, Lrp5, β-catenin, and Snail, as well as the administration of YS49 was a focal point (FIG. 20C).

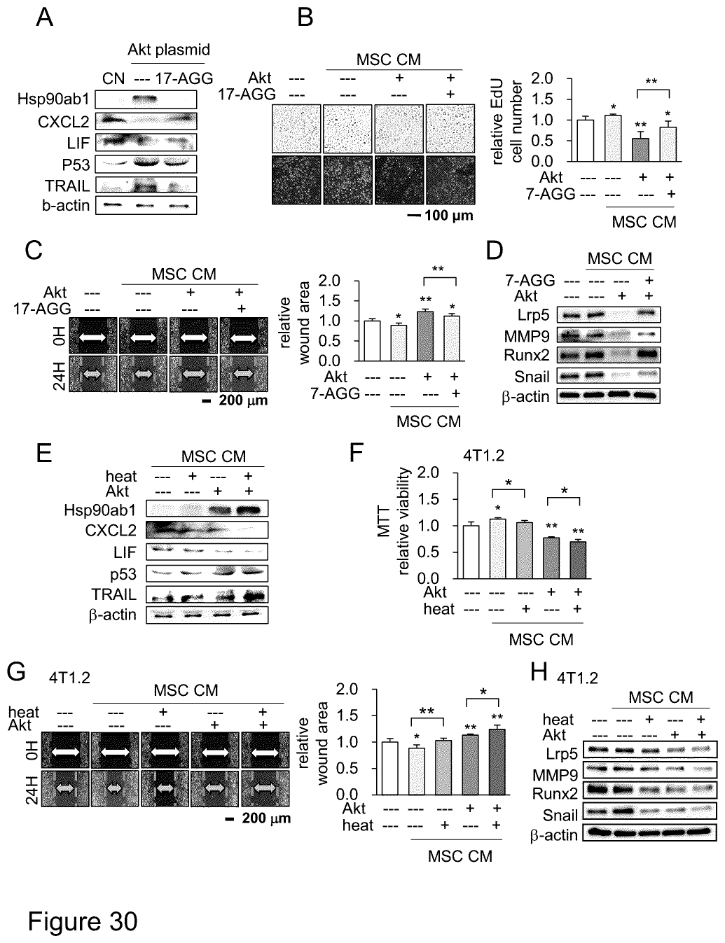

Hsp90ab1 as an extracellular tumor suppressor. Hsp90ab1 reduced the scratch-based migration and downregulated tumor-promoting genes (Lrp5, MMP9, Runx2, and Snail) in 4T1.2 cells (FIGS. 20D and 20E). By contrast, Hsp90ab1-silenced MSC CM yielded the opposite outcome (FIGS. 20F-20H). In Hsp90ab1-silenced MSC CM, the expression profile showed the elevation of CXCL2 and LIF and the reduction in p53 and Trail (FIG. 201). Furthermore, MSC CMs treated with siRNAs specific for Akt, Lrp5, β-catenin, or Snail did not show the elevation of Hsp90ab1, calreticulin, and peptidylprolyl isomerase B (FIG. 29B). Of note, the administration of HSF1, heat shock protein 1, reduced the MTT-based viability of 4T1.2 cells, but Hsp90aa1 recombinant protein did not alter the levels of Lrp5, MMP9, Runx2, and Snail in 4T1.2 cells (FIGS. 29C and 29D). The pharmacological inhibitor of Hsp90, Tanespimycin (17-AGG), blocked the tumor-suppressing capability of Hsp90ab1, and the incubating MSCs at 42° C. for 1 h elevated the level of Hsp90ab1 and induced heat shock-driven generation of induced tissue-specific stem (“iTS”) MSCs (FIG. 30).

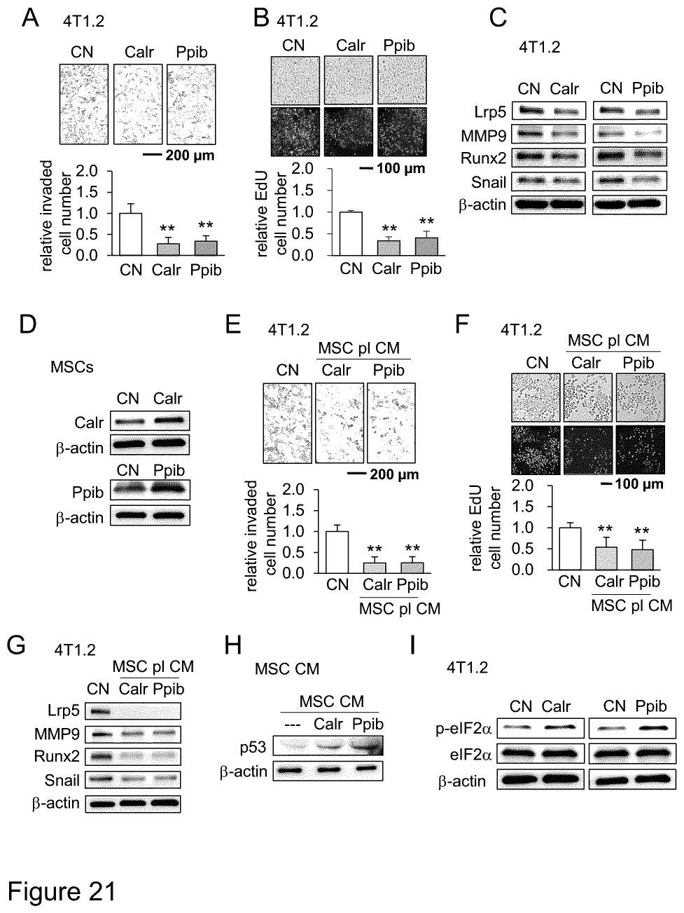

Calreticulin (Calr), and peptidylprolyl isomerase B (Ppib) as tumor suppressors. Calr and Ppib are proteins in the endoplasmic reticulum (ER) and facilitate protein folding and assembly. Similar to using Hsp90ab1, addition of extracellular Calr and Ppib significantly inhibited TRANSWELL® invasion and EdU-based proliferation of 4T1.2 cells (FIGS. 21A and 21B). The expression of Lrp5, MMP9, Runx2, and Snail in 4T1.2 cells was also suppressed by extracellular Calr and Ppib (FIG. 21C). Notably, Calr- and Ppib-overexpressing MSC CMs significantly reduced TRANSWELL® invasion and EdU-based proliferation of 4T1.2 cells (FIGS. 21D-21F). Furthermore, these MSC CMs downregulated Lrp5, MMP9, Runx2, and Snail (FIG. 21G). The elevation of p53 in Calr- and Ppib-overexpressing MSC CMs (FIG. 21H) was also detected. Stress to the ER elevates the phosphorylation level of eukaryotic translation initiation factor 2 alpha (eIF2a). Interestingly, the application of Calr and Ppib recombinant proteins to 4T1.2 mammary tumor cells elevated the phosphorylation level of eIF2α (FIG. 21I).

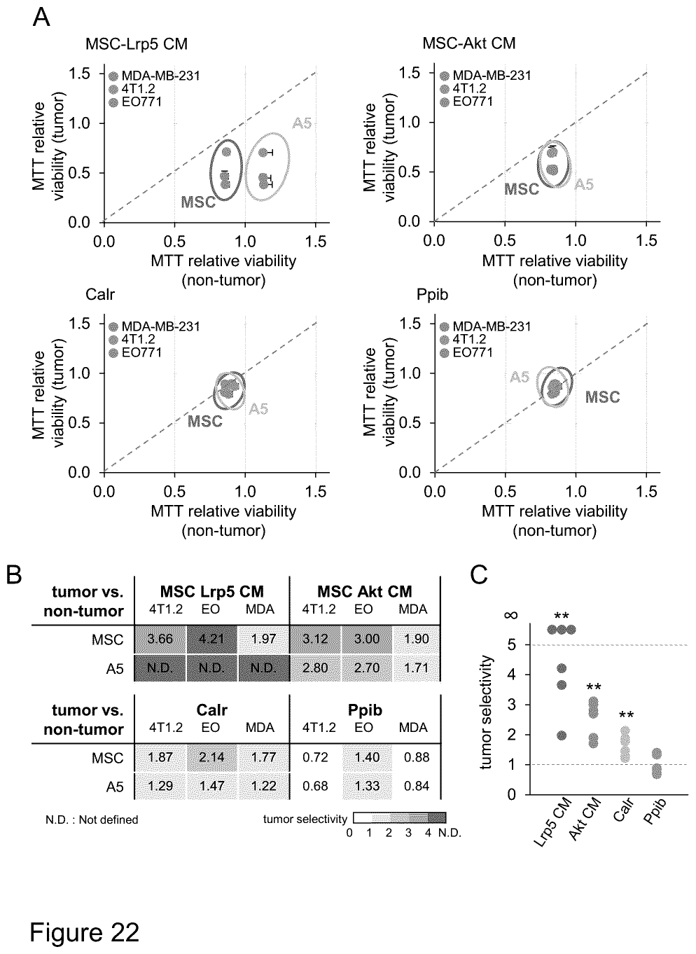

Tumor-selective inhibition by MSC CMs and Calr. Desirably MSC CMs and tumor-suppressing protein candidates should only inhibit the progression of tumor cells and not non-tumor cells. The inhibitory ratio was defined sing MTT-based viability as (reduction in MTT-based viability of tumor cells)/(reduction in MTT-based viability of non-tumor cells). A value larger than 1 indicates that the inhibition is stronger to tumor cells than non-tumor cells. Using three tumor cells (MDA-MB-231, 4T1.2, and EO771) and two non-tumor cells (MSCs, and MLO-A5 osteocytes), tumor selectivity for Lrp5 CM, Akt CM, Calr, and Ppib (FIG. 22) was determined. The highest tumor selectivity was obtained with Lrp5 CM, in which the MTT-based viability of A5 osteocytes was stimulated. Akt CM also presented the tumor selectivity of 2.41±0.70. Between Calr and Ppib, Calr gave a higher tumor selectivity than Ppib.

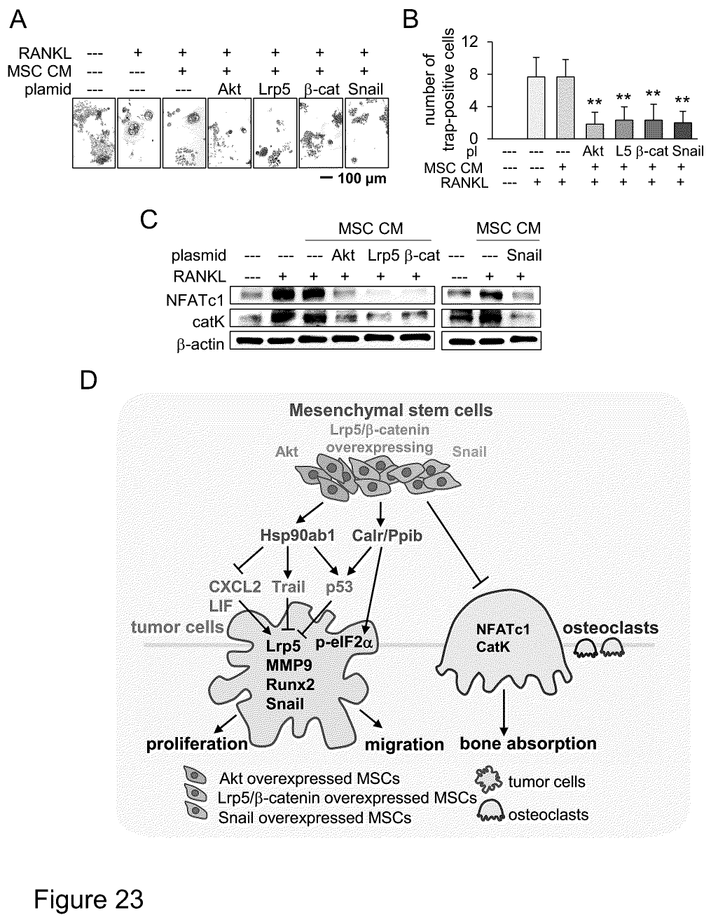

Inhibition of osteoclast development by MSC CM. In the tumor-invaded bone, it is important to inhibit the development of osteoclasts that resorb bone. The effect of MSC CM on the maturation and gene expression of osteoclasts was examined. Besides tumor suppression, the results showed that MSC CMs markedly inhibited the development of RANKL-stimulated RAW264.7 pre-osteoclasts by the overexpression of Akt, Lrp5, β-catenin, and Snail (FIGS. 23A and 23B). The levels of NFATc1 and cathepsin K were significantly reduced by these MSC CMs (FIG. 23C). Taken together, the result supports the inhibitory action of MSC CM for not only tumor progression but also the development of osteoclasts.

### Example 3

Cell culture. EO771 mammary tumor cells (CH3 BioSystems, Amherst, NY, USA) and astrocytes (Cell biologics, Chicago, IL, USA) were grown in DMEM (Corning, Inc., Corning, NY, USA), MLO-A5 osteocyte-like cells (obtained from Dr. L. Bonewald at Indiana University, IN, USA), and bone marrow-derived MSCs (harvested from C57BL/6 mice) were cultured in αMEM (Gibco, Carlsbad, CA, USA). The culture media were supplemented with 10% fetal bovine serum and antibiotics (50 units/ml penicillin, and 50 μg/ml streptomycin; Life Technologies, Carlsbad, CA, USA), and cells were incubated at 37° C. with 5% CO₂.

CM was prepared from MLO-A5 osteocytes and astrocytes at ˜80% confluence after 24 h incubation and an Amicon filter unit with a cutoff mass at 3 kDa (Sigma-Aldrich, St. Louis, MO, US) was used to remove microparticles and condense it by 10-fold. Cellular proliferation was examined using an MTT assay, and a wound-healing scratch assay and a TRANSWELL® invasion assay were conducted to evaluate cell motility and invasion capability, respectively, using the procedure previously described.

Spheroid assay, plasmid transfection, and Western blot analysis. For the spheroid assay, 1.0×10⁴ cells/well were cultured in a U-bottom low-adhesion 96-well plate (S-Bio, Hudson, NH, USA) for 24 h. Lrp5 plasmids (#115907, Addgene, Watertown, MA, USA), β-catenin plasmids (#31785, Addgene), and IL-1ra plasmids (RG218518, Origene, Rockville, MD, USA), were transfected using pcDNA as control. In Western blot analysis, cells were lysed in a radio-immunoprecipitation assay buffer and isolated proteins were size-fractionated and electro-transferred. Antibodies against Snail, TGFβ, Lrp5, Runx2, Caspase 3 (cas3), cleaved Caspase 3 (c-cas3), histone H4 (Cell Signaling), p53, CXCL2, IL1ra (Invitrogen, Carlsbad, CA, USA), MMP9 (Santa Cruz Biotechnology, Dallas, Texas, USA), TRAIL (Novus Biologicals, Centennial, CO, USA), LIMA1 (Novus, Centennial, CO, USA), DSP (Proteintech, Rosemont, IL, USA), CXCL5 (Abcam, Cambridge, MA, USA), and β-actin (Sigma-Aldrich) were used. RNA interference was conducted to silence histone H4 (Hist4h4 Select, Life Technologies), together with a nonspecific negative control siRNA (SILENCER SELECT™ #1, Life Technologies). Cells were transiently transfected with siRNA with LIPOFECTAMINE™ RNAiMAX (Life Technologies), and the medium was replaced by a regular culture medium after 24 h.

Skull diffusion assay. The usage of human breast cancer tissues was approved by the Indiana University Institutional Review Board, and informed consent for research use was obtained from all patients. The experiment was performed following Indiana University's human research protection program policies. A breast cancer tissue (— 1 g; ER/PR+, HER2+), received from Simon Cancer Center Tissue Procurement Core, was manually fragmented with a scalpel into small pieces. Mouse skulls were isolated and rinsed with PBS. They were placed in a 24-well plate with the head top facing the well surface and tumor cells or breast cancer tissue fragments were grown. The well was filled with the control medium or osteocyte-derived CM. Fluphenazine (Sigma-Aldrich), a dopamine receptor antagonist that suppresses the growth of mammary tumor cells, was employed as a positive control to suppress tumor growth.

Estimation of the diffusion coefficient through the skull. Suppose that the two aqueous reservoirs are separated by a skull and the solute (albumin) in the first reservoir with the volume V₁ is transferred to the second reservoirs with the volume V₂. When the volume of the skull is negligible, the change in the solute in the first reservoir is approximated, using the quasi-steady-state flux, N according to Eq. (1) and Eq. (2):

$$- {V}_{1} \frac{{dc}_{1} ( t )}{dt} = A \cdot N \qquad Eq . ( 1 )$$

$$N = D \frac{{c}_{1} ( t ) - {c}_{2} ( t )}{L} \qquad Eq . ( 2 )$$

Where t=time, and c 1 (t) and c 2 (t)=concentrations in the first and second reservoirs, respectively, A=skull surface area, D=diffusion coefficient through the skull, and L=skull thickness. Since the decrease in the first reservoir is equivalent to the gain in the second reservoir, Eq. (3) is arrived at:

$${V}_{1} \frac{{dc}_{1} ( t )}{dt} = - A \cdot D \frac{{c}_{1} ( t ) - {c}_{2} ( t )}{L} \qquad Eq . ( 3 )$$

If the initial concentrations are c₁(t=0)=c₀ and c₂ (t=0)=0, the concentration c₁ is predicted as a function of time according to Eq. (4):

$$ln \frac{{c}_{1} ( t ) - \frac{{V}_{1}}{{V}_{1} + {V}_{2}} {c}_{0}}{\frac{{V}_{2}}{{V}_{1} + {V}_{2}} {c}_{0}} = - \frac{A \cdot D}{L {V}_{1}} ( 1 + \frac{{V}_{1}}{{V}_{2}} ) t \qquad Eq . ( 4 )$$

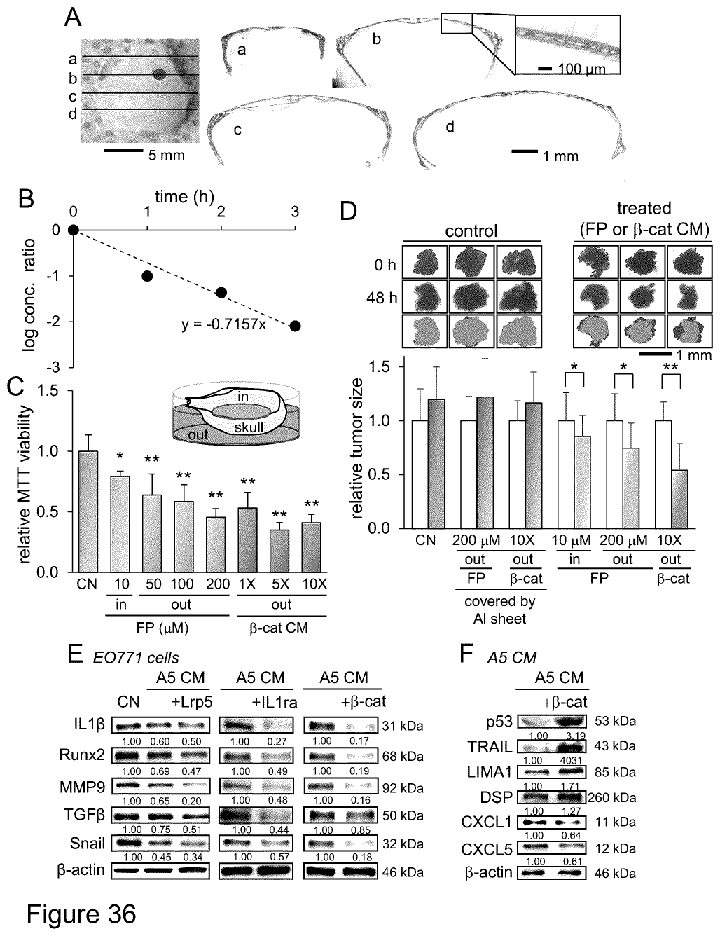

The diffusion coefficient of albumin in the skull was estimated as 3.30×10⁻⁸ cm²/s from the slope (0.7157) of the best-fit line of Eq. (4) in FIG. 36B. Of note, V₁=265 μl, V₂=65 μl, c₀=10 μg/μ1, A=188 mm², and L=60 μm.

Animal model. The procedures were approved by the Indiana University Animal Care and Use Committee and complied with the Guiding Principles in the Care and Use of Animals endorsed by the American Physiological Society. The sample size was decided using power analysis and stratified randomization was applied based on body weight. The mice were sacrificed on day 14, whereas the mice that died before day 7 were excluded.

To evaluate the effects of osteocytes or osteocyte-derived CM on mammary tumors or tumor-invaded tibiae, C57BL/6 female mice (˜8 weeks; 18 mice per group) were randomly divided into three cell-testing groups (placebo, and osteocyte injection with and without Lrp5 overexpression), as well as two CM-testing groups (placebo, and β-catenin-overexpressing osteocyte-derived CM). Ten mice received the injection of EO771 cells (3.0×10⁵ cells in 50 μl PBS) to the mammary fat pad on day 0, while eight mice to the proximal tibia. The cell-testing groups received the co-injection of osteocytes (1.5×10⁵ cells) on day 0, while the CM-testing groups were given daily 50 μl of 10-fold condensed β-catenin CM subcutaneously to the mammary fat pad or systemically from the tail vein from day 2 to 14. The placebo group received the same volume of PBS.

To evaluate tumor progression in the brain, C57BL/6 female mice (˜8 weeks; 8 mice per group) were divided into five cell-testing groups, including the placebo, osteocyte group, and Lrp5-, IL1ra, and Lrp5-/IL1ra-overexpressing osteocyte groups, as well as three CM-testing groups (placebo, osteocyte-derived CM with and without (3-catenin overexpression). All mice received the stereotaxic injection of EO771 cells (1.0×10⁴ cells in 15 μl PBS) into the right side of the frontal lobe on day 0, using a method previously reported. The cell-testing groups received the injection of osteocytes (3.0×10⁴ cells in 15 μl PBS) on days 0 and 7. The CM-testing groups received 50 μl of osteocyte-derived CM with and without β-catenin overexpression subcutaneously to the right parietal daily from day 2 to 14. The placebo group received the same volume of PBS.

Activity score and brain damage score. The activity score was determined blindly using the method previously described with minor modifications. The score was in the range of 0 to 10 (10 for most active), with the score contributions for general appearance (0 to 2), natural behavior (0 to 3), provoked behavior (0 to 3), and body conditions (0 to 2). The brain damage score in the range of 0 to 10 was determined blindly based on 5 phenotypic features (surface roughness, bleeding, cortex swelling, cortex asymmetry, and encephalomalacia). Each feature was scored from 0 to 2, with the best brain damage score of 0.

X-ray imaging and histology. A whole-body X-ray image was taken using the Faxitron radiographic system (Faxitron X-ray Co., Tucson, AZ, USA). The tibiae were scored blindly at levels 0 to 3 as a bone damage score, in which 0=normal; 1=clear bone boundary with slight periosteum proliferation; 2=bone damage and moderate periosteum proliferation; and 3=severe bone erosion. Brain samples were fixed in 4% paraformaldehyde in PBS for 24 h. They were dehydrated through a series of graded alcohols, cleared in xylene, and embedded in paraffin. The samples were sliced coronally with 4.5 μm thickness and H&E staining was conducted and analyzed in a blinded fashion.

Whole-genome proteomics: Proteins in CM were analyzed in the Dionex UltiMate 3000 RSLC system combined with the Q-exactive high-field hybrid quadrupole orbitrap mass spectrometer (Thermo Fisher Scientific). Proteins were first digested on-beads using trypsin/LysC. Digested peptides were desalted and separated using a trap and 50-cm analytical columns. Raw data were processed using MAXQUANT™ (v1.6.3.3) against the Uniprot mouse protein database at a 1% false discovery rate allowing up to 2 missed cleavages. MS/MS counts were used for relative protein quantitation and proteins identified with at least 1 unique peptide and 2 MS/MS counts were considered for the final analysis. To evaluate the tumor-suppressing capability of the predicted candidates, seven recombinant proteins such as Hspa8 (heat shock protein family A member 8), Vim (vimentin), (NBP1-30278, NBP2-35139; Novus, Littleton, CO, USA), Hsp90ab1 (heat shock protein 90 alpha family class B member 1) (OPCA05157; Aviva system biology, San Diego, CA, USA), histone H4, Ubc (Ubiquitin), Ppia (peptidylprolyl isomerase A), Flna (filamin A) and Ncl (nucleolin) (MBS2097677, MBS2029484, MBS286137, MBS962910, MBS146265; MyBioSource, San Diego, CA, USA) were employed. In the MTT assay, 5 μg/ml of each of these recombinant proteins were added and viability of tumor cells was evaluated.

Statistical analysis: The data were expressed as mean±S.D. Two independent samples and paired-samples were analyzed using Student's t-test and paired t-test, respectively. For three independent experiments, statistical significance was evaluated using a one-way analysis of variance (ANOVA). Post hoc statistical comparisons among the groups were performed using Bonferroni correction with statistical significance at p<0.05. The single and double asterisks in the figures indicate p<0.05 and p<0.01, respectively.

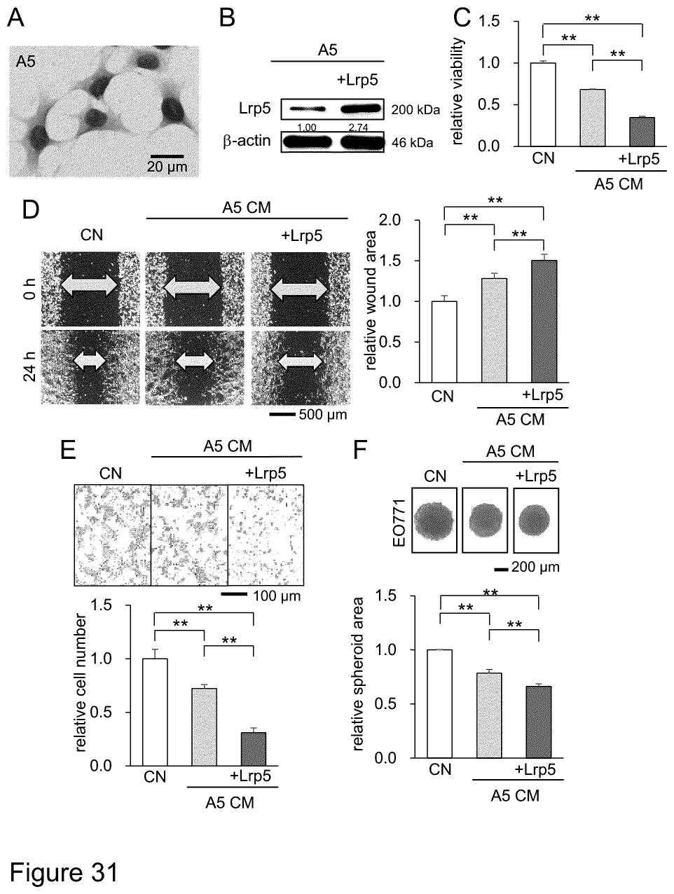

Suppression of viability, migration, and invasion of EO771 tumor cells by osteocyte-derived CM. The effect of Lrp5-overexpressing osteocytes in the tumorigenic behaviors of EO771 mammary tumor cells, using MLO-A5 osteocytes, was evaluated (FIGS. 31A and 31B). The result showed that A5-derived CM significantly decreased MTT-based viability of tumor cells (FIG. 31C), inhibited their migration and invasion (FIGS. 31D and 31E), and suppressed the growth of tumor spheroids (FIG. 31F). Notably, the observed anti-tumor effect was enhanced by the overexpression of Lrp5 in osteocytes (FIGS. 31C-31F). Taken together, the in vitro analysis revealed that osteocyte-derived CM presented the tumor-suppressing capability and the anti-tumor action was enhanced by Lrp5 overexpression.

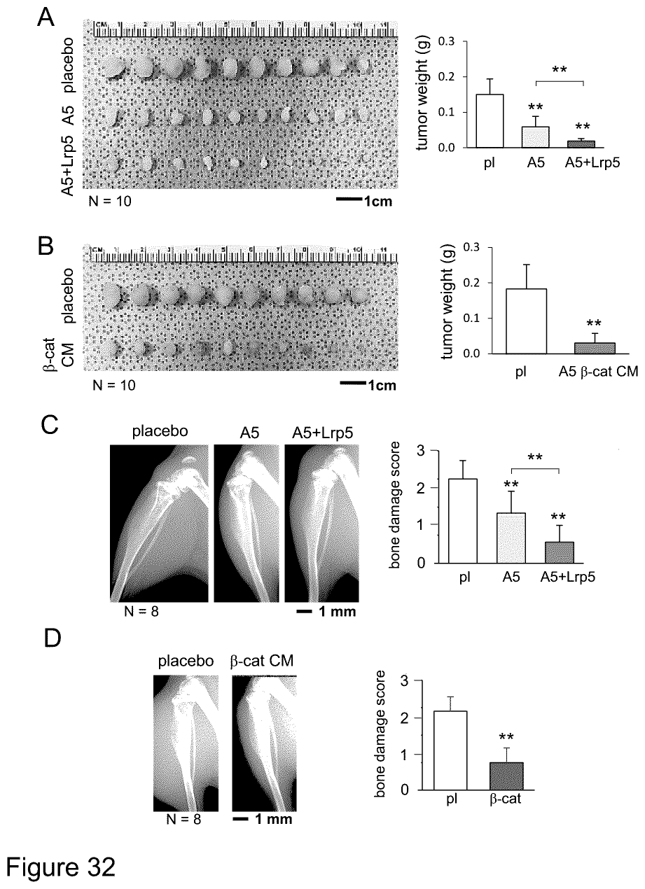

Before examining the effect of osteocyte-derived CM in brain tumors, its action on the progression of mammary tumors and tumor-invaded bone degradation was evaluated. In a mouse model of mammary tumors and tibial osteolysis using C57BL/6 female mice, the co-injection of osteocytes to the mammary fat pad significantly reduced the size of mammary tumors and the anti-tumor effect was strengthened by the overexpression of Lrp5 (FIG. 32A). Furthermore, the daily administration of β-catenin overexpressing CM markedly shrank mammary tumors (FIG. 32B). The inhibitory effect was also observed in the tumor-invaded tibia. X-ray images showed that the co-injection of osteocytes with and without Lrp5 overexpression as well as the systemic administration of β-catenin-overexpressing CM suppressed tumor-driven bone loss (FIGS. 32C and 32D).

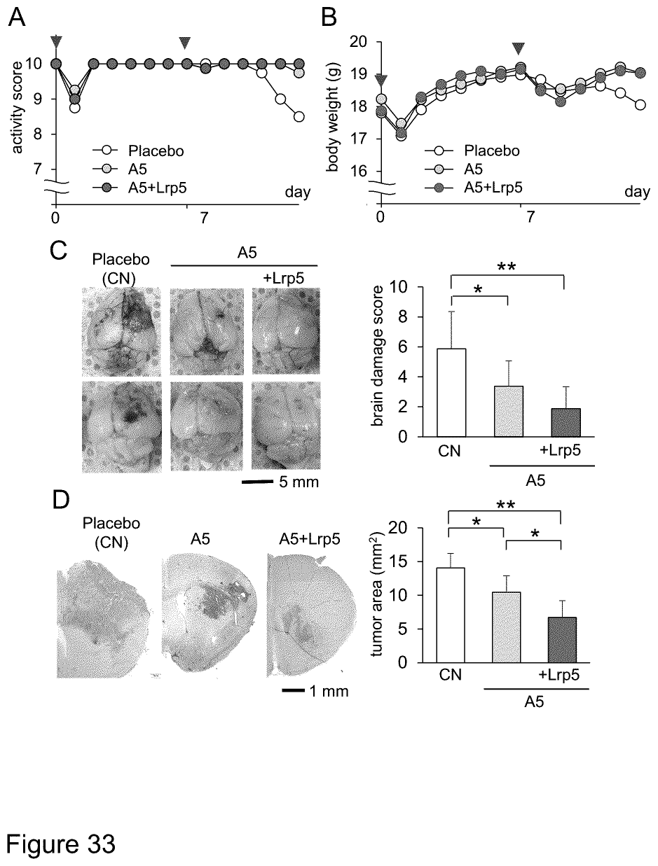

Suppression of tumor growth in the brain by the co-injection of osteocytes. Since in vivo analyses of mammary tumors in the mammary fat pad and tibia showed the inhibitory effect of osteocyte-derived CM, the action of osteocytes on brain tumors on the right side of the frontal lobe was next evaluated. Among the three groups, the A5 group received the co-injection of osteocytes, while the A5+Lrp5 group received the co-injection of Lrp5-overexpressing osteocytes. The activity scores, body weight, and brain damage scores were the worst in the placebo group (FIGS. 33A-C), whereas they were the best in the A5+Lrp5 group (FIG. 33C). In the histological analysis, the progression of brain tumors was significantly suppressed in the A5 group and the overexpression of Lrp5 in the A5+Lrp5 group further reduced the tumor-invaded area in the brain (FIG. 33D). Collectively, the co-injection of osteocytes into the brain suppressed the growth of brain tumors and the overexpression of Lrp5 in osteocytes enhanced the anti-tumor action.

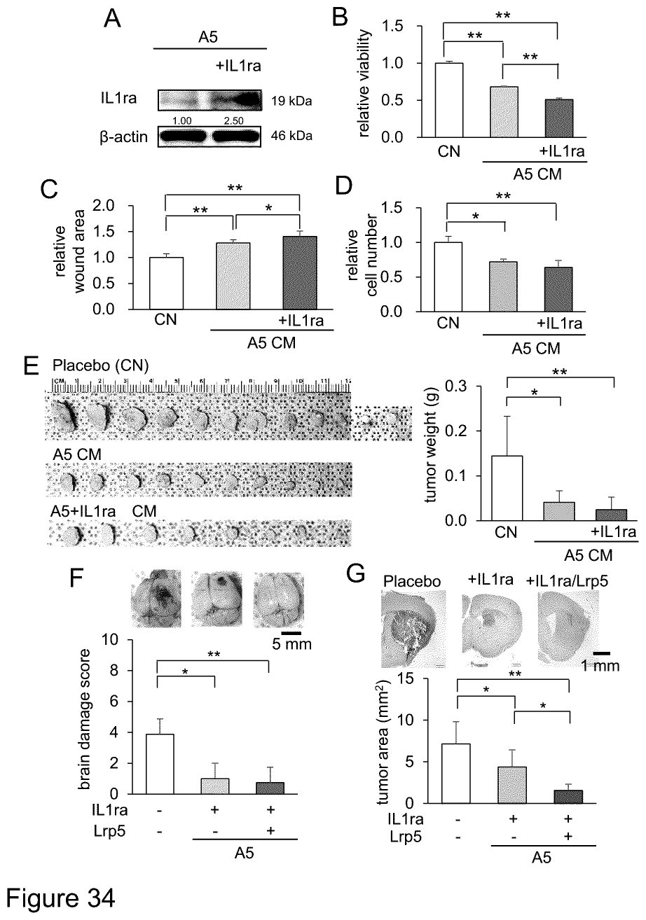

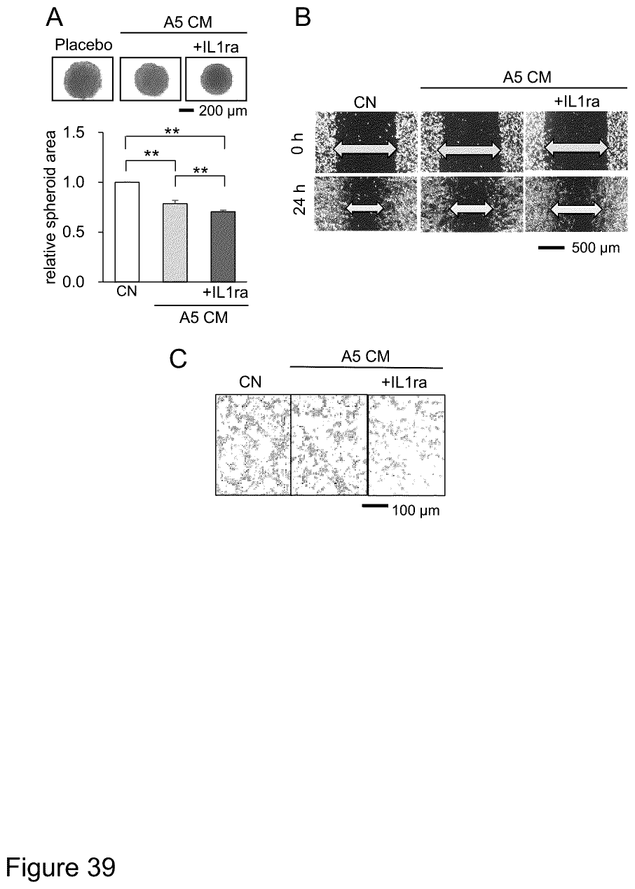

Anti-tumor effects of IL1ra overexpression on brain tumors. So far, it was shown that Lrp5-overexpressing osteocytes and their CM suppressed the progression of tumors in the mammary fat pad, bone, and brain. The role of IL1ra (FIG. 34A) was next evaluated. In the MTT, scratch-based migration, and TRANSWELL® invasion assays, IL1ra-overexpressing CM presented significantly stronger anti-tumor effects than osteocyte-derived CM without its overexpression (FIGS. 34B-34D; FIGS. 39A-39C). In a mouse model of mammary tumor in which EO771 cells were inoculated into the mammary fat pad, the co-injection of IL1ra-overexpressing osteocytes markedly reduced the size and weight of tumors (FIG. 34E). Taken together, the results reinforced the anti-tumor action of osteocytes and revealed that besides the overexpression of Lrp5 or β-catenin, the anti-tumor effect was also enhanced by the overexpression of IL1ra.

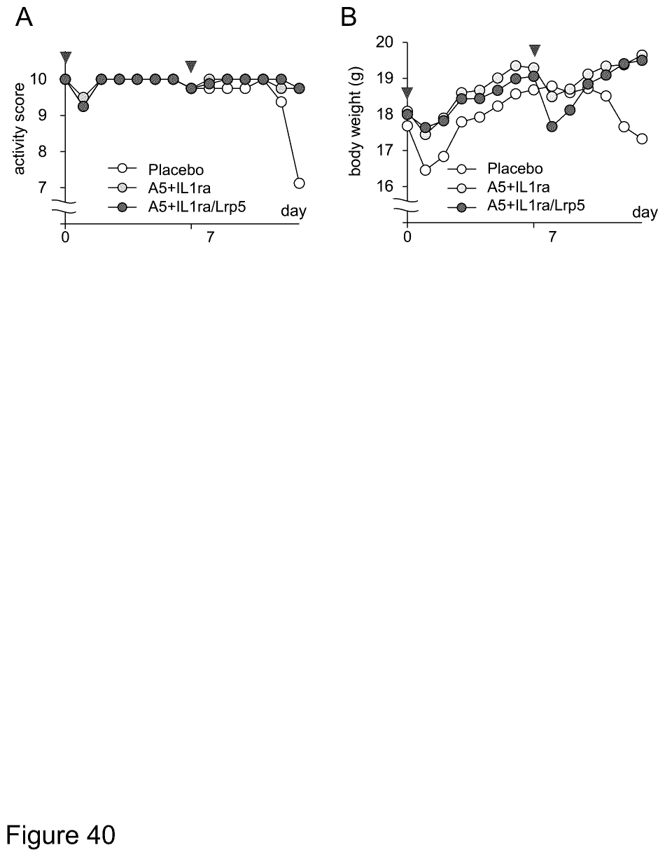

To further evaluate the action of IL1ra- and Lrp5-overexpressing osteocytes, IL1ra- and Lrp5 were co-transfected in osteocytes. In the IL1ra group and the (IL1ra+Lrp5) group, the activity score and body weight were superior to those in the placebo group (FIGS. 40A and 40B), as well as the brain damage scores (FIG. 34F). In the histological analysis, the size of brain tumors in the (IL1ra+Lrp5) group was the smallest (FIG. 34G). The result thus indicated that the combined overexpression of IL1ra and Lrp5 further enhanced the osteocyte-driven anti-tumor effect.

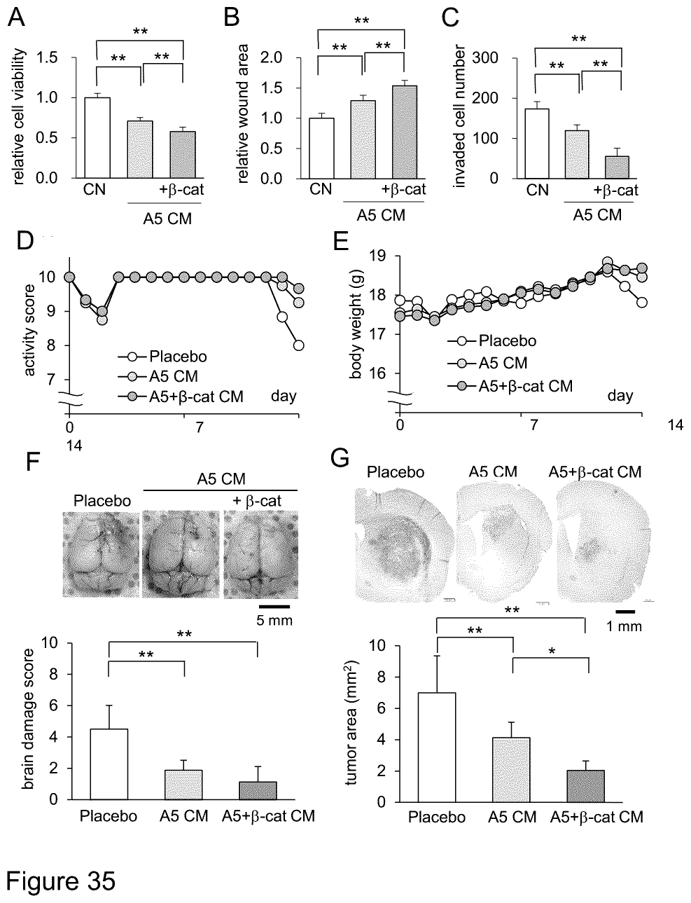

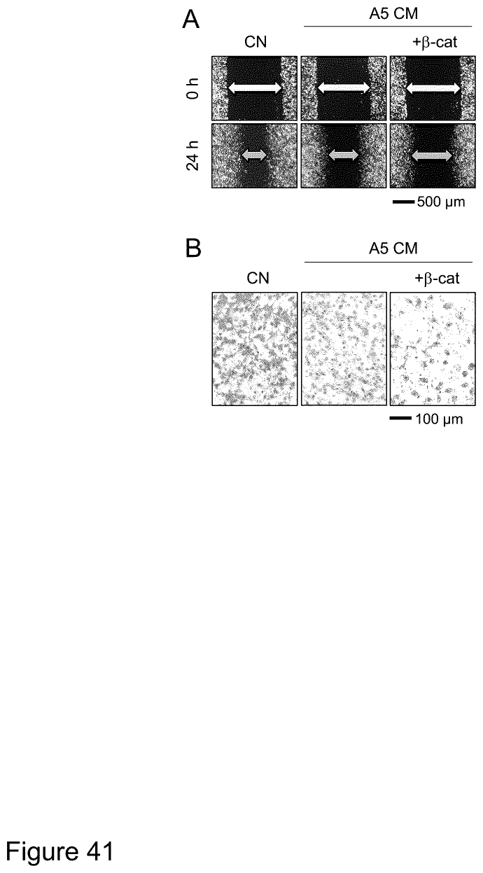

Anti-tumor effect of β-catenin overexpressing CM on brain tumors. Instead of introducing osteocytes, which are not associated with brain-resident cells, using the needle-based injection through the skull, the minimally-invasive application of β-catenin overexpressing CM as a subcutaneous injection was next examined. In vitro analyses with the MTT, scratch-based migration, and TRANSWELL® invasion assays revealed that β-catenin-overexpressing CM significantly suppressed the proliferation, migration, and invasion of tumor cells (FIG. 35A-35C; FIGS. 41A and 41B). In the mouse model of brain tumors, the administration of β-catenin-overexpressing CM to the head skin resulted in better activity scores, higher body weights, and improved brain damage scores than the placebo (FIGS. 35D-35F). Histological analysis revealed that the growth of brain tumors was significantly reduced by the application of osteocyte-derived CM and the overexpression of β-catenin further reduced the tumor-invaded area in the brain (FIG. 35G). No harmful side-effects, such as bleeding, inflammation, and infection, were observed at the site of CM administration. The in vivo result herein indicated that the minimally invasive injection of β-catenin-overexpressing CM was effective to inhibit the growth of brain tumors.

Transport analysis of CM through the skull. While the suppression of tumor growth in the brain by the administration of osteocyte-derived CM suggested the transport of tumor-suppressing factors into the brain through the skull, the biophysical property of the skull was examined and the diffusive transport phenomena was characterized. The coronal histological sections showed that the murine skull consisted of a porous microstructure without any obvious penetrating hole (FIG. 36A). The diffusion coefficient of bovine serum albumin (BSA) from the outside of the skull to its inside was estimated. The temporal changes in the logarithmic concentration ratio (see Eq. (4) above) were plotted as a function of time (FIG. 36B). The diffusion coefficient of BSA across the skull is linked to the slope of the plots and it was estimated to be 3.30×10⁻⁸ cm²/s. The estimated value across the skull is approximately 20-fold smaller than that in the aqueous solution.

To further evaluate the transport across the skull, the MTT-based viability assay was next conducted, in which EO771 cells were placed in the inside of the skull, while anti-tumor agents such as Fluphenazine and β-catenin CM in its outside. The result showed that compared to the control, both Fluphenazine and β-catenin CM significantly decreased the viability of tumor cells in the inside of the skull (FIG. 36C). Furthermore, the same transport analysis using freshly isolated human breast cancer fragments was conducted. The result also revealed that the administration of Fluphenazine and β-catenin CM in the outside of the skull significantly shrunk the tumor fragments in the inside of the skull (FIG. 36D). Collectively, these in vitro and ex vivo analyses revealed that tumor-suppressing factors in CM can be transported across the skull.

Enrichment of tumor suppressors in osteocyte-derived CM. The tumor-suppressing action of osteocyte-derived CM to the mammary fat pad, bone, and brain indicates that tumor-suppressing proteins are enriched in osteocyte-derived CM and this enrichment is amplified by the overexpression of Lrp5, β-catenin, and IL1ra. Before examining potential tumor suppressors, the levels of IL113, Runx2, MMP9, TGFβ, and Snail in EO771 cells were found to be downregulated by osteocyte-derived CM, and their levels were further reduced by the overexpression of the three selected genes (FIG. 36E). In β-catenin-overexpressing CM, the levels of four known tumor suppressors (p53, TRAIL, LIMA1, DSP) were upregulated, whereas the levels of two tumor-promoting chemokines (CXCL1 and CXCL5) were downregulated (FIG. 36F).

Extracellular histone H4 as a novel tumor suppressor in CM. Besides the above tumor suppressors, a whole-genome proteomics analysis was conducted, identifying fifty-six proteins that were enriched in β-catenin-overexpressing CM (Table 2). Table 2 includes a summary of the tumor-suppressing protein candidates (56 proteins) by the whole-genome proteomics analysis. Compared to the control medium, these proteins were enriched in the β-catenin-overexpressing osteocyte-derived condition medium.

TABLE 2

| | Mol. Wt. | Expression Level (MS/MS Count) | | |
|---|---|---|---|---|
| Gene Name | (kDa) | CN | βcat | Bcat-CN |
| Hspa8 | 70.90 | 17 | 44 | 27 |
| Hsp90ab1 | 83.28 | 7 | 28 | 21 |
| Hist2h4; Hist1h4 | 11.37 | 2 | 17 | 15 |
| Ubc | 17.23 | 5 | 19 | 14 |
| Ppia | 17.97 | 0 | 14 | 14 |
| Flna | 280.47 | 0 | 13 | 13 |
| Vim | 53.69 | 0 | 13 | 13 |
| Ncl | 76.86 | 0 | 13 | 13 |
| Hist1h2bj; Hist1 | 13.58 | 0 | 12 | 12 |
| Actg1 | 41.79 | 6 | 17 | 11 |
| Aldoa | 39.36 | 0 | 10 | 10 |
| Eno1 | 47.14 | 4 | 13 | 9 |
| Calm1 | 16.84 | 6 | 14 | 8 |
| Pgam1 | 28.83 | 2 | 10 | 8 |
| Eefla1 | 50.11 | 0 | 8 | 8 |
| Lmna | 74.24 | 0 | 8 | 8 |
| Ldha | 36.50 | 0 | 8 | 8 |
| Msn | 67.74 | 0 | 8 | 8 |
| Sh3d21 | 26.85 | 17 | 24 | 7 |
| Pkm | 57.84 | 0 | 7 | 7 |
| Tpm3; Tpm3-rs | 29.02 | 0 | 7 | 7 |
| Hist1h2ah; Hist1 | 13.66 | 3 | 9 | 6 |
| Tubb5 | 49.67 | 0 | 6 | 6 |
| Eef2 | 95.26 | 0 | 6 | 6 |
| Ywhaz | 27.77 | 0 | 6 | 6 |
| Tpi1 | 32.19 | 3 | 8 | 5 |
| Gm20390; Nme | 30.20 | 3 | 8 | 5 |
| Tubalb; Tubalc | 50.15 | 0 | 5 | 5 |
| Atp5b | 56.30 | 0 | 5 | 5 |
| Eif4a1 | 46.02 | 0 | 5 | 5 |
| Ywhae | 29.19 | 5 | 9 | 4 |
| Hsp90aa1 | 84.79 | 0 | 4 | 4 |
| Actn4 | 104.98 | 0 | 4 | 4 |
| Hspa4 | 94.21 | 0 | 4 | 4 |
| Ppib | 23.71 | 0 | 4 | 4 |
| Hspa5 | 72.42 | 2 | 5 | 3 |
| Myh9 | 226.37 | 0 | 3 | 3 |
| Atp5a1 | 59.75 | 0 | 3 | 3 |
| Vcp | 89.32 | 0 | 3 | 3 |
| Npm1 | 29.52 | 0 | 3 | 3 |
| Tkt | 67.63 | 0 | 3 | 3 |
| Eef1g | 50.06 | 0 | 3 | 3 |
| Prdx1 | 22.18 | 0 | 3 | 3 |
| Eif5a; Eif5a2 | 16.30 | 0 | 3 | 3 |
| Prdx6 | 24.87 | 0 | 3 | 3 |
| GAPDH; Gapdh | 35.81 | 0 | 3 | 3 |
| Sod1 | 15.94 | 9 | 11 | 2 |
| Cts1 | 37.56 | 3 | 5 | 2 |
| Pgk1 | 44.55 | 0 | 2 | 2 |
| Hnrnpa2b1 | 37.40 | 0 | 2 | 2 |
| Got2 | 47.41 | 0 | 2 | 2 |
| Actn1 | 103.07 | 0 | 2 | 2 |
| Calu | 37.06 | 0 | 2 | 2 |
| Fasn | 272.41 | 0 | 2 | 2 |
| Myl6 | 16.96 | 0 | 2 | 2 |
| Calr | 42.20 | 2 | 3 | 1 |

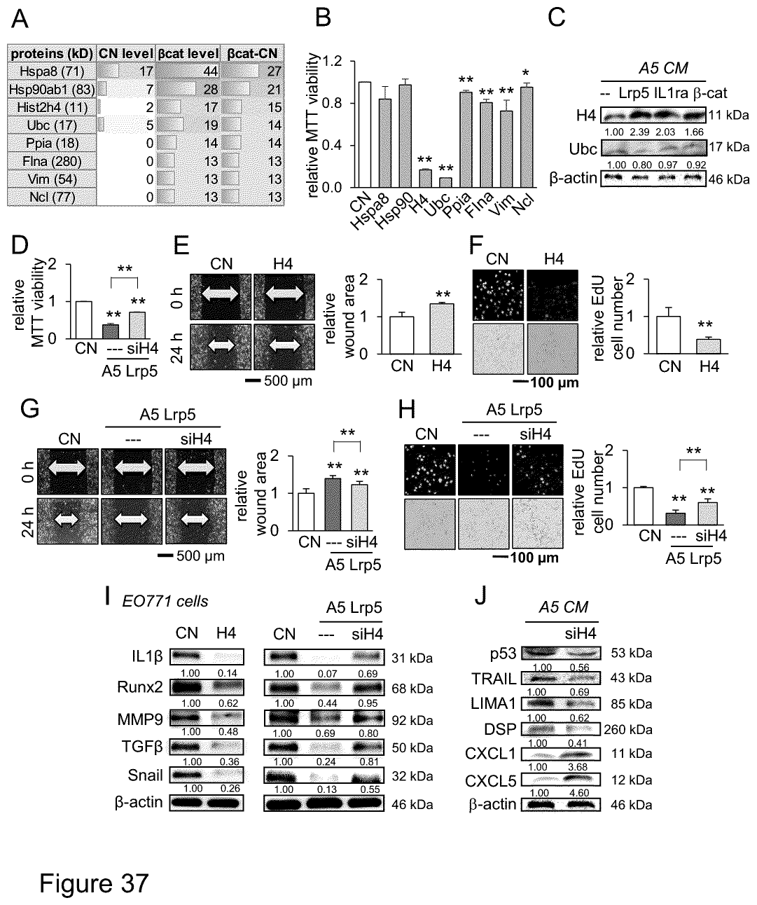

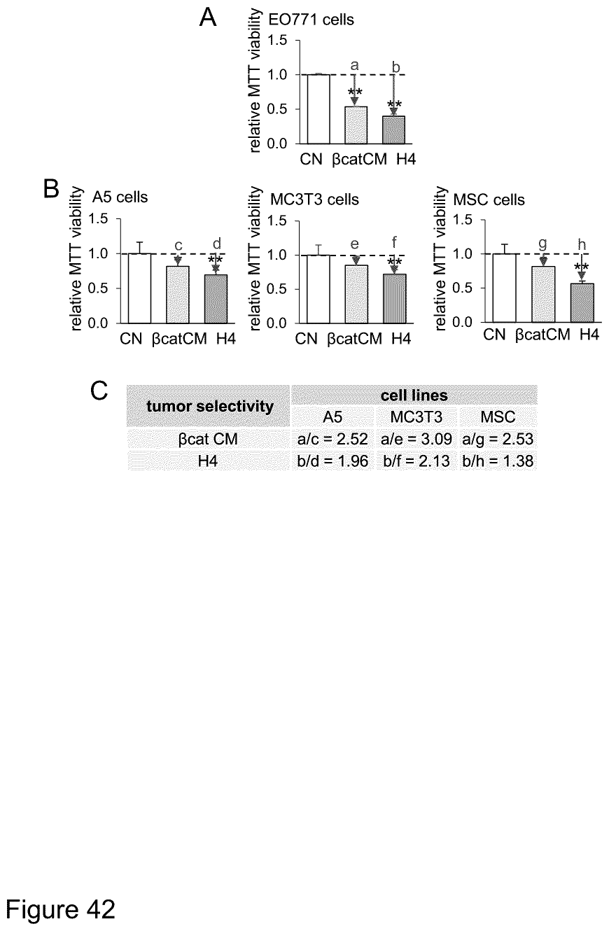

The effect of the top eight candidates was examined in the MTT assay (FIG. 37A). The result revealed that six out of eight candidates significantly inhibited the viability of EO771 mammary tumor cells (FIG. 37B). In particular, histone H4 (H4) and ubiquitin C (Ubc) exhibited striking inhibition and the elevated level of histone H4 in Lrp5-, IL1ra-, and β-catenin-overexpressing osteocyte-derived CM, but not Ubc, were observed in Western blotting, (FIG. 37C). In the MTT assay, the inhibitory action of histone H4 is stronger to tumor cells than to non-tumor cells (FIG. 42). RNA interference with histone H4 siRNA suppressed the inhibitory effect of Lrp5-overexpressing CM (FIG. 37D). The treatment with histone H4 reduced the scratch-based migration and EdU-based proliferation (FIGS. 37E and 37F), while the reduction was suppressed by histone H4 siRNA (FIGS. 37G and 37H). Also, histone H4 decreased the levels of IL1β, Runx2, MMP9, TGFβ, and Snail in EO771 cells, while their decrease was reversed by the H4 siRNA-treated CM (FIG. 371). Last, the partial silencing of histone H4 downregulated the selected tumor suppressors such as p53, TRAIL, LIMA1, and DSP, and upregulated the tumor promoters such as CXCL1 and CXCL5 (FIG. 37J).

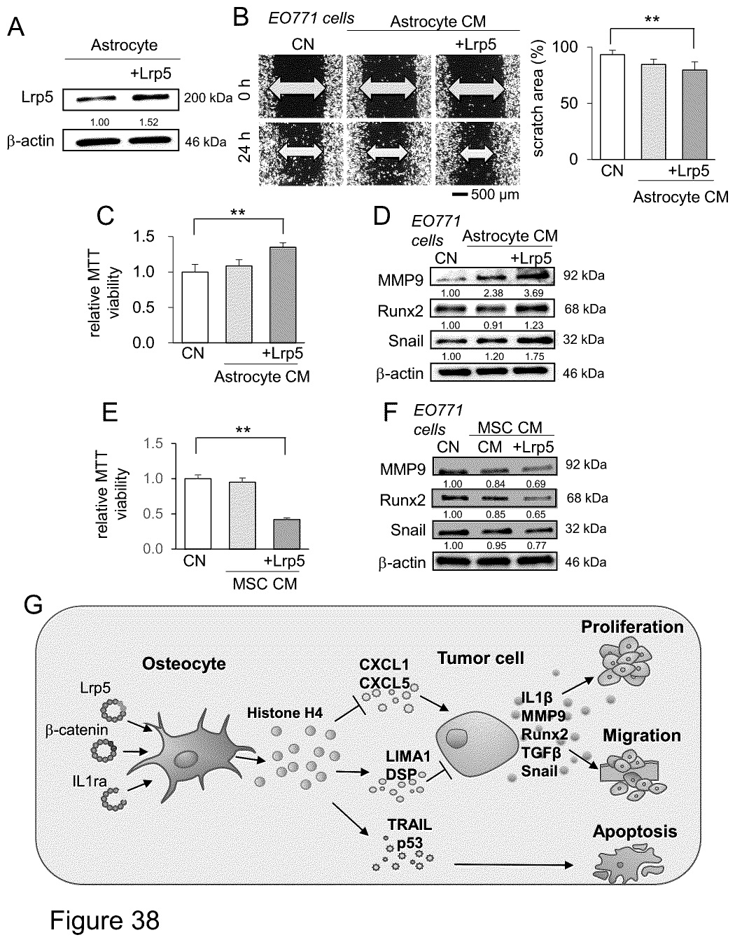

Differential effects of astrocyte- and MSC-derived CM. Besides osteocytes, the effects of astrocytes and MSCs were evaluated. Astrocytes are the most abundant glial cells in the brain, while MSCs are commonly utilized in regenerative medicine. In the MTT-based viability and scratch-based migration assays, the astrocyte-derived CM did not induce any detectable change (FIGS. 38A-38C). In contrast to Lrp5-overexpressing osteocytes, Lrp5-overexpressing astrocyte-derived CM promoted the viability and migration of EO771 tumor cells, and the level of MMP9, Runx2, and Snail in EO771 cells were upregulated (FIG. 38D). By contrast, MSCs did not present the innate anti-tumor actions but the overexpression of Lrp5 converted them into anti-tumor agents by reducing the MTT-based viability and downregulating MMP9, Runx2, and Snail (FIGS. 38E and 38F).

### Example 4

Osteocytes Cell culture. TRAMP-C2ras prostate tumor cells, EO771 mouse mammary tumor cells (CH3 BioSystems, Amherst, NY, USA), and 4T1.2 mouse mammary tumor cells (obtained from Dr. R. Anderson at Peter MacCallum Cancer Institute, Melbourne, Australia) were cultured in DMEM. MDA-MB-231 breast cancer cells (ATCC), MLO-A5 osteocytes (obtained from Dr. L. Bonewald at Indiana University, IN, USA), RAW264.7 pre-osteoclast cells were grown in αMEM. PC-3 human prostate cancer cells (ATCC) were cultured in RPMI-1640 (Gibco, Carlsbad, CA, USA). The culture media was supplemented with 10% fetal bovine serum and antibiotics (50 units/ml penicillin, and 50 μg/ml streptomycin), and cells were maintained at 37° C. and 5% CO₂. Recombinant TRAIL proteins (BioLegend, San Diego, CA, USA) were administered to tumor cells.

EdU assay, TRANSWELL® invasion assay, and scratch assay. Cellular proliferation was examined using a fluorescence-based cell proliferation kit (Thermo-Fisher, Waltham, MA, USA). After fluorescent labeling, the number of fluorescently labeled cells was counted and the ratio to the total number of cells was determined. A TRANSWELL® invasion assay was conducted to detect cell motility, and a wound-healing scratch assay was utilized to evaluate 2-dimensional cell motility.

Western blot analysis. Cultured cells were lysed in a radio-immunoprecipitation assay buffer with protease inhibitors (Santa Cruz Biotechnology, Santa Cruz, CA, USA) and phosphatase inhibitors (Calbiochem, Billerica, MA, USA). The protein concentration was determined using a BCA Protein Assay kit (Thermo-Fisher). Proteins were fractionated by 10-15% SDS gels and electro-transferred to polyvinylidene difluoride transfer membranes (Millipore, Billerica, MA, USA). After blocking 1 h with a blocking buffer (Bio-Rad), the membrane was incubated overnight with primary antibodies and then with secondary antibodies conjugated with horseradish peroxidase for 45 min (Cell Signaling, Danvers, MA, USA). The level of proteins was determined using a SUPERSIGNAL™ west femto maximum sensitivity substrate (Thermo Scientific, Waltham, MA, USA), and a luminescent image analyzer (LAS-3000, Fuji Film, Tokyo, Japan) was used to quantify signal intensities. Antibodies against cleaved caspase 3 (9661S), Lrp5 (5731S), Snail (3879S), TGFβ (3711S), cathepsin K (sc-48353), MMP9 (sc-393859), NFATc1 (sc-7294), TRAIL (NB500-220), LIMA1 (C91753) (Novus, Centennial, CO, USA), ANXA6 (ab199422) (Abcom, Cambridge, United Kingdom), p53 (MA5-12557), (Invitrogen, Carlsbad, CA, USA), and β-actin (Sigma, Saint Louis, MO, USA) were used.

Plasmid transfection, RNA interference, and cytokine analysis. Using 2×10⁶ cells, Lrp5 and p53 plasmids (40 ng/μ1) were transfected. Conditioned medium (CM, 9 ml) was prepared with antibiotics and a fraction of FBS consisting of factors with 3 kDa or smaller. After one day incubation, the medium was ultra-centrifuged to remove exosomes and condensed 10-fold by filtering (Amicon, Sigma, Saint Louis, MO, USA) to collect proteins larger than 3 kDa. For overexpressing Lrp5 (#115907, Addgene, Watertown, MA, USA) and p53 (#69003, Addgene), A5 osteocytes and RAW264.7 pre-osteoclasts were transfected with their plasmids, while a blank plasmid vector (FLAG-HA-pcDNA3.1; Addgene) was used as a control. Osteocytes were treated with shRNA specific to Lrp5 (sc-149050-V, Santa Cruz), with control GFP shRNA (sc-108084, Santa Cruz). Cells were transfected with LIPOFECTAMINE®3000 (Thermo, L300015), and stable transfectants were selected using PUROMYCIN™ (Sigma). Using a LIPOFECTAMINE™ RNAiMAX transfection reagent (Thermo, 13778100), osteocytes and RAW264.7 cells were also transfected with TRAIL siRNA (Thermo, S75440), using the negative control siRNA (Thermo, 4390843).

3D spheroid assay and ex vivo tissue assay. Cells were cultured in Ultra-low attachment 96-well plates (S-BIO, New Hampshire, USA) at 1×10⁴ cells/well for TRAMP cells. Cells were imaged every 24 h, and the area was calculated with IMAGE J. In the ex vivo tissue assay, the usage of human prostate cancer tissues was approved by the Indiana University Institutional Review Board. A sample (— 1 g), received from Simon Cancer Center Tissue Procurement Core, was manually fragmented with a scalpel into small pieces (0.5˜0.8 mm in length). These pieces were grown in DMEM with 10% fetal bovine serum and antibiotics for a day. Lrp5-overexpressing osteocyte-derived CM was then added for two additional days, and changes in the fragment size were determined.

FRET imaging. To evaluate tension force at a focal adhesion and migratory capacity of tumor cells in response to Lrp5-overexpressing osteocyte-derived CM, a plasmid expressing a vinculin tension sensor (VinTS, #26019, Addgene) was transfected. The fluorescence lifetime images were acquired by a custom-made microscope built on a laser scanning confocal microscope (FLUOVIEW™ 1000, Olympus; Center Valley, PA, USA), using the procedures previously described. Of note, a decrease in the tension force of the vinculin sensor implies an increase in fluorescence efficiency.

Animal models. The experimental procedures using animals were approved by the Indiana University Animal Care and Use Committee and complied with the Guiding Principles in the Care and Use of Animals endorsed by the American Physiological Society. C57BL/6 mice lacking Lrp5 in osteocytes (Dmp1-Cre; Lrp5f/f; conditional Lrp5 knockout mice) were created by breeding Dmp1-Cre transgenic mice with Lrp5 floxed mice, both of which have been described earlier. Mice were housed five per cage and provided with mouse chow and water ad libitum.

In the mammary tumor model, C57BL/6 female mice (˜8 weeks, Envigo RMS, Inc., Indianapolis, IN, USA) received subcutaneous injections of TRAMP cells (3.0×10⁵ cells in 50 μl PBS) to the mammary fat pad. The animals were sacrificed on day 18, and the weight of each tumor was measured. In the mouse model of osteolysis, the samples/animals were randomly allocated to experimental groups and processed for blind evaluation. Ten C57BL/6 male mice per group received an injection of TRAMP cells (3.0×10⁵ cells in 20 μl PBS) into the right tibia as an intra-tibial injection. Osteocytes with and without LRP5 overexpression were co-injected at the mammary fat pad or the proximal tibia together with TRAMP cells. Roughness was determined by drawing a line along the bone and adding the areas of the bone extruded from the line and the areas of the non-bone on the other side of the line. In the extravasation assay, EO771 cells were labeled with a green fluorescent dye and injected with and without iTS cell-derived CM via a lateral tail vein. Mice were sacrificed after 48 h for histological identification of extravascular tumor cells in the lung.

X-ray, μCT imaging, and histology. A whole-body X-ray image was taken using the Faxitron radiographic system (Faxitron X-ray Co., Tucson, AZ, USA). The tibia was harvested for μCT imaging and histology. Micro-computed tomography was performed using SKYSCAN™ 1172 (Bruker-MicroCT, Kontich, Belgium). Using manufacturer-provided software, scans were performed at pixel size 8.99 μm, and the images were reconstructed and analyzed (CTAN™ v1.13). In histology, H&E staining was conducted as described previously.

Statistical analysis. For cell-based experiments, three or four independent experiments were conducted, and data were expressed as mean±SD. In animal experiments, the sample size in the mouse model was chosen to achieve a power of 80% with p<0.05. The primary experimental outcome was tumor weight for the mammary fat pad experiment and the bone volume ratio (BV/TV) for the tibia experiment. The secondary experimental outcome was tumor size for the mammary fat pad experiment and the trabecular number (Tb.N) for the tibia experiment. All data were tested for normality (Shapiro-Wilk normality test) before statistical tests. Statistical significance was evaluated using a one-way analysis of variance (ANOVA). Post hoc statistical comparisons with control groups were performed using Bonferroni correction with statistical significance at p<0.05. A nonparametric Kolmogorov-Smirnov test was applied to evaluate FRET efficiency in live-cell imaging. The single and double asterisks in the figures indicate p<0.05 and p<0.01, respectively.

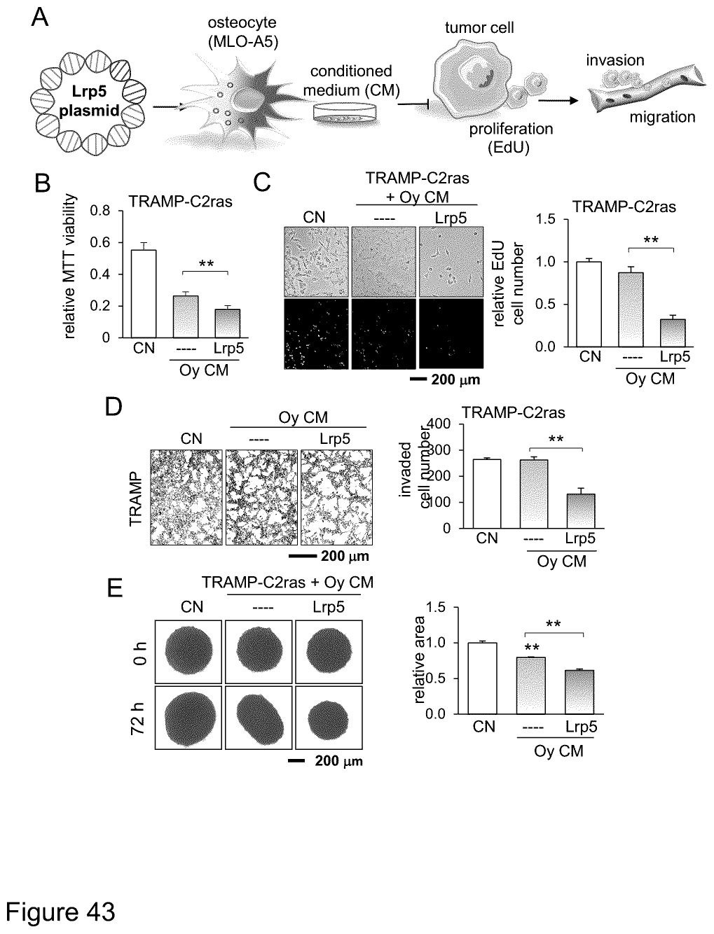

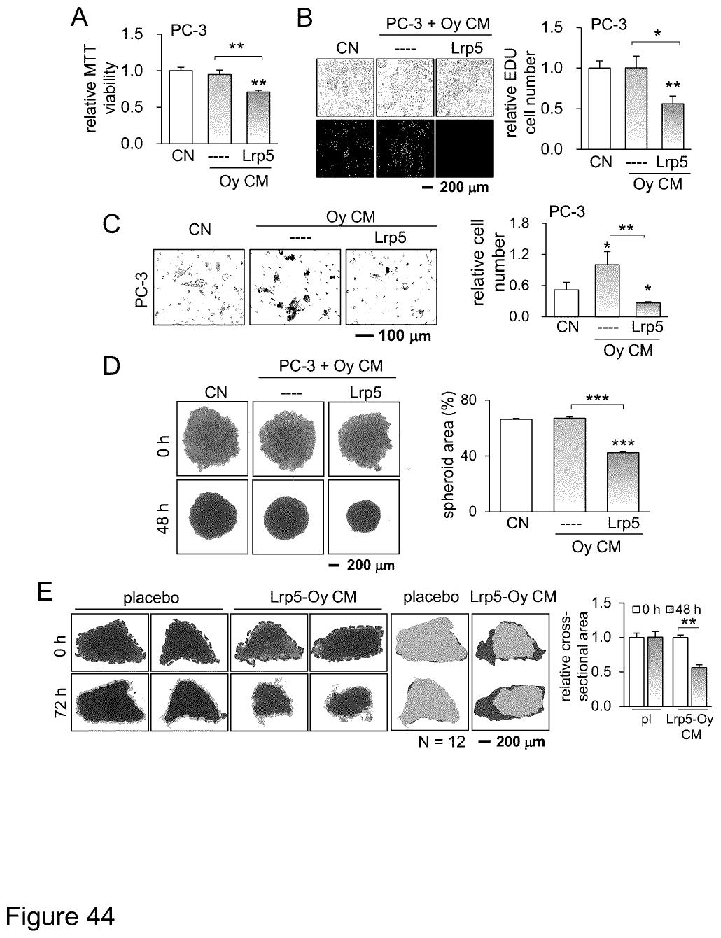

Inhibition of the proliferation and invasion by Lrp5-CM. The response of TRAMP-C2ras (TRAMP) prostate tumor cells to MLO-A5 osteocyte-derived CM (CM) was examined. In the MTT-based viability, EdU-based proliferation, and TRANSWELL® invasion assays, CM did not significantly alter tumor cell behaviors (FIGS. 43A-43D). When Lrp5 was overexpressed, however, Lrp5-CM reduced the viability, proliferation, and invasion of TRAMP cells. CM slightly but significantly inhibited the growth of 3-dimensional tumor spheroids, and the overexpression of Lrp5 enhanced the growth inhibition (FIG. 43E). Consistent with the response of TRAMP cells, the inhibitory effects of Lrp5-CM were also detected in PC-3 human prostate tumor cells in the EdU-based proliferation, TRANSWELL® invasion, and tumor spheroid assays (FIGS. 44A-44D).

Besides the tumor-suppressing capability of Lrp5-CM in prostate cancer cells, freshly isolated human prostate cancer tissues were employed to correlate with clinical significance. The tissue was manually fragmented and the fragments were cultured in Lrp5-CM. The result revealed that compared to the placebo (control CM), the Lrp5-CM group significantly reduced the size of cancer fragments (FIG. 44E).

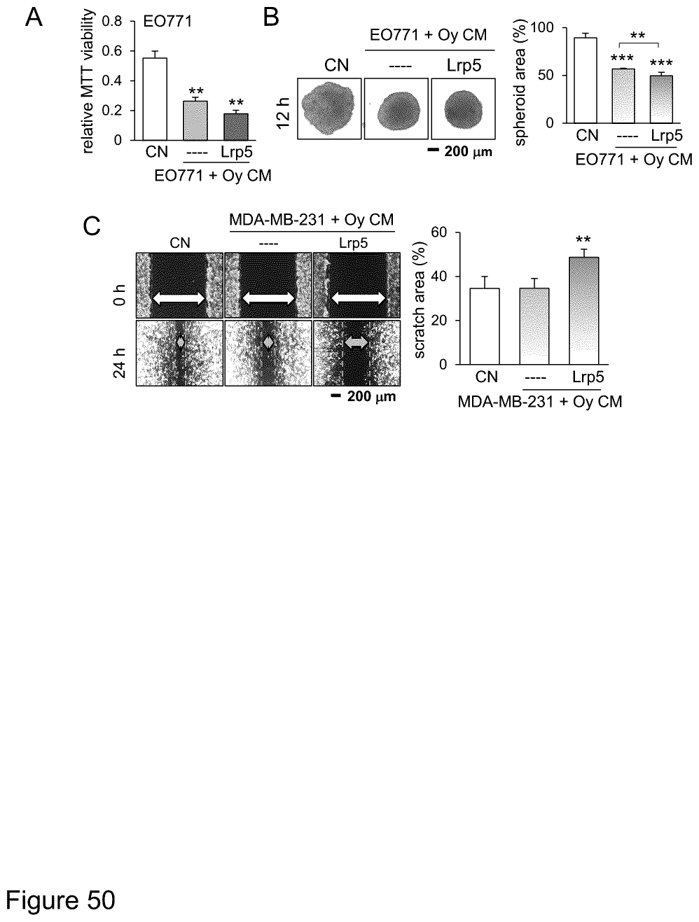

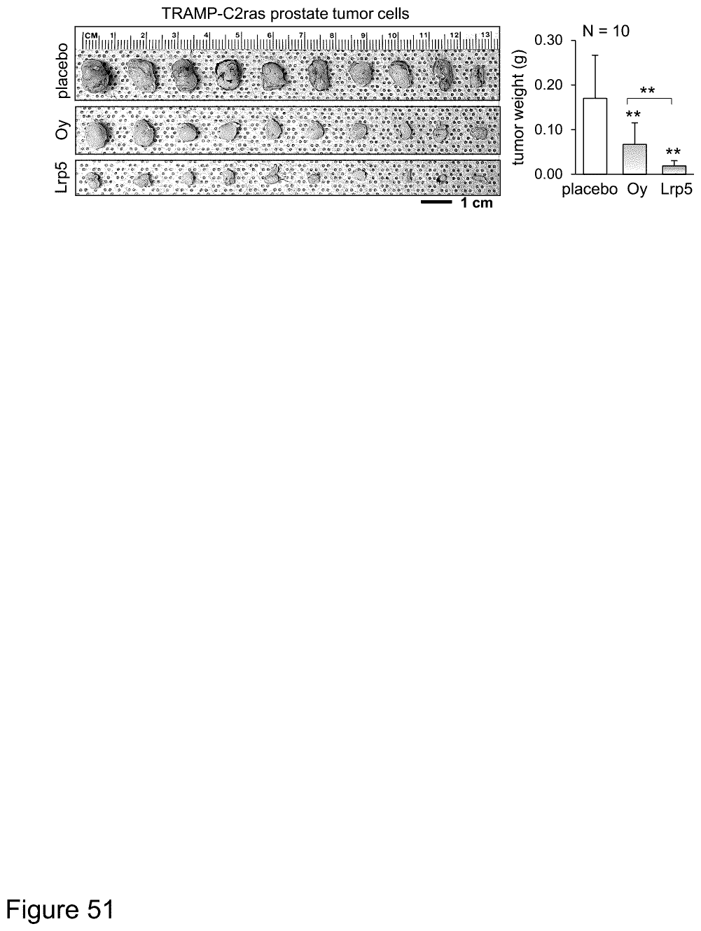

To compare the effects of Lrp5-CM between prostate and breast cancer cells, EO771 mammary tumor cells, and MDA-MB-231 breast cancer cells were exposed to CMs. Consistently, the overexpression of Lrp5 inhibited the proliferation and migration of EO771 and MDA-MB-231 cells (FIGS. 50A-50C). To further evaluate the efficacy of Lrp5-Oy, TRAMP cells were injected into the mammary fat pad of female C57BL/6 mice. Notably, the weight of tumor tissues was significantly reduced by a local injection of Lrp5-Oy (FIG. 51).

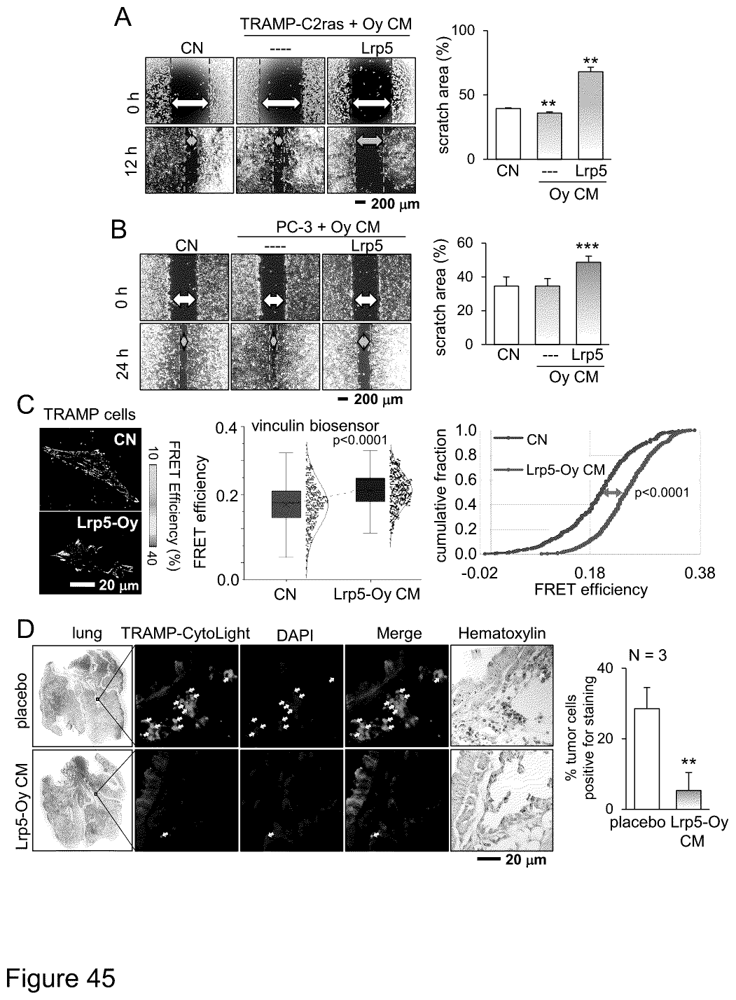

Inhibition of the migration and extravasation by Lrp5-CM. The effect of Lrp5-CM on the in vitro migration and in vivo extravasation of tumor cells was examined. In the scratch-based migration assay, Lrp5-CM reduced the migration of TRAMP and PC-3 cells (FIGS. 45A and 45B). Consistent with the suppression of 2-dimensional cell motility, fluorescent live-cell imaging showed that the FRET efficiency of the vinculin biosensor was increased by Lrp5-CM in TRAMP cells (FIG. 45C). The observed increase implies a decrease in vinculin-mediated molecular force at focal adhesions, in agreement with the inhibition of migratory behaviors. Furthermore, the intravenous injection of Lrp5-CM with TRAMP cells markedly reduced the number of tumor cells invaded into the lungs of C57BL/6 male mice (FIG. 45D).

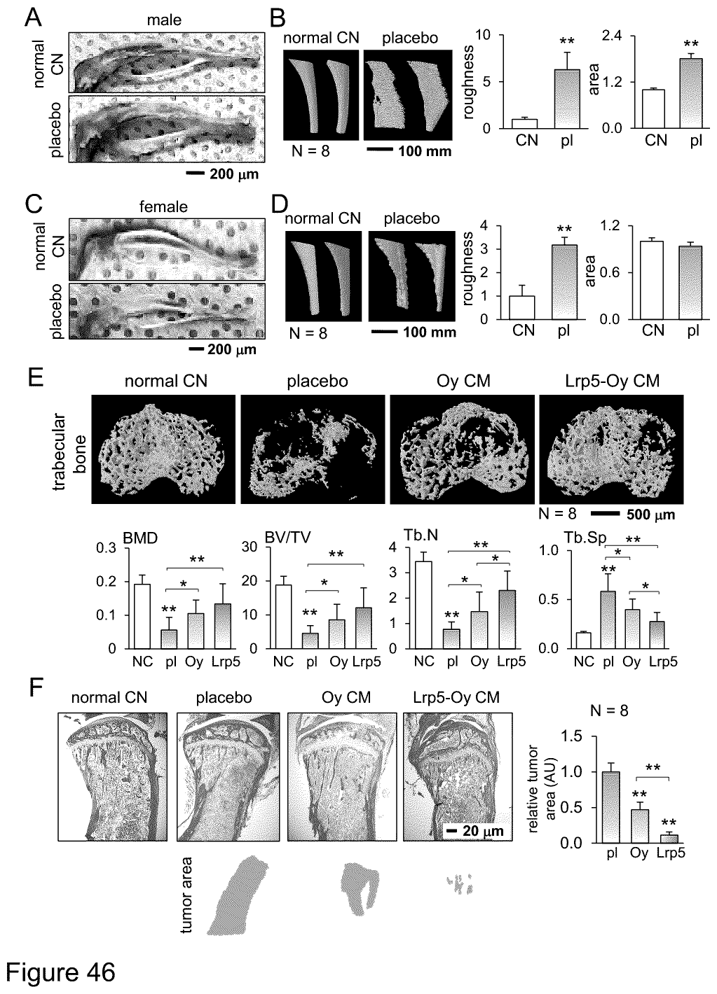

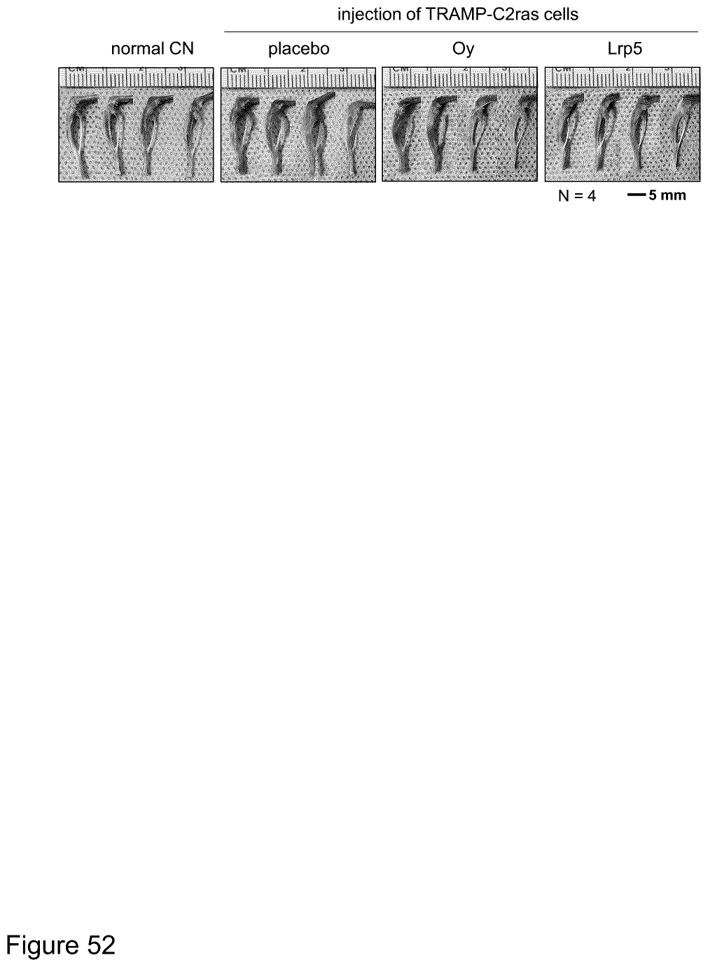

Inhibition of the tumor progression in the tibia by Lrp5-Oy. In the male and female mouse models, tumor cells invaded into the tibia by the inoculation of TRAMP and EO771 cells, respectively. In the TRAMP cell-invaded tibia and fibula, the tibia was swollen and the fibula was enlarged with an increase in surface roughness (FIGS. 46A and 46B; FIG. 52). In response to the inoculation of EO771 cells in female mice, however, the tibia was not swollen and the fibula was not enlarged although the surface roughness of the fibula was elevated (FIGS. 46C and 46D).

Three-dimensional μCT reconstruction of the proximal tibia and histological examination supported the tumor-suppressing action of Lrp5-Oy in male mice. Compared to the placebo that received the inoculation of TRAMP cells alone, the Oy and Lrp5-Oy injected groups reduced trabecular bone loss in the proximal tibia with the elevation of bone mineral density, bone volume ratio, and trabecular number, with a decrease in trabecular separation (FIG. 46E). H&E-stained histological sections revealed that the tumor-invaded area was markedly reduced by the local injection of Oy and Lrp5-Oy (FIG. 46F).

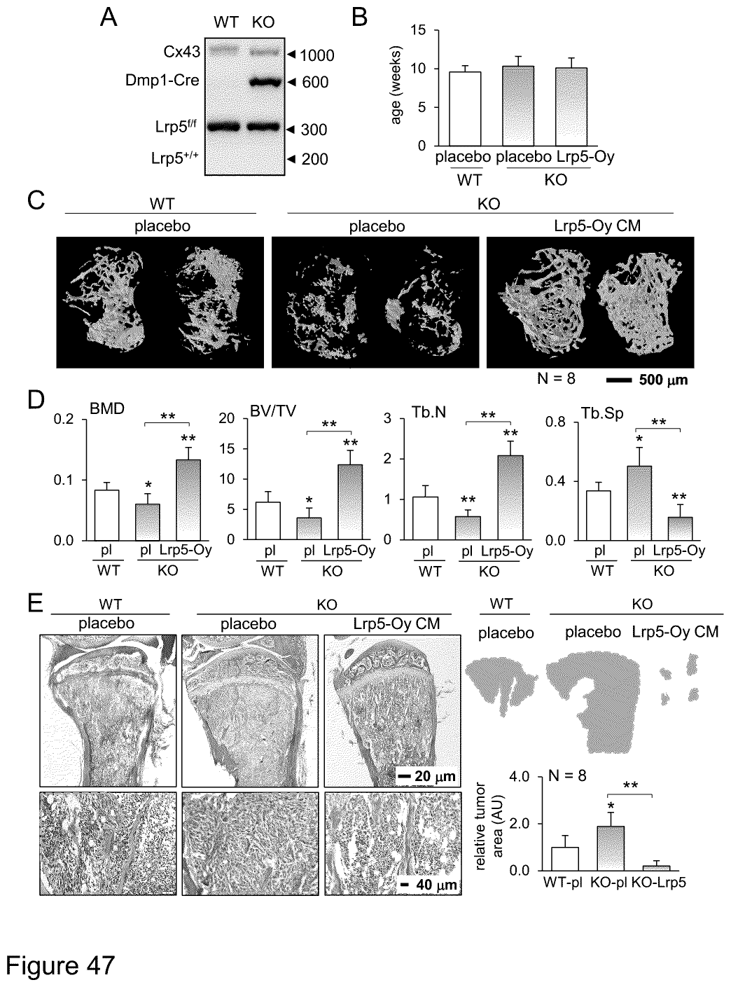

Increased tumor-driven bone loss in Lrp5-deleted mice. Consistent with the tumor-suppressing effects by the overexpression of Lrp5, Lrp5-deleted conditional knockout mice showed a more severe bone loss in trabecular bone in the TRAMP cell-inoculated tibia than their wildtype littermates (FIGS. 47A-47C). Furthermore, the injection of Lrp5-CM to the proximal tibia significantly improved the bone microstructure, with an increase in bone mineral density, bone volume ratio, and the trabecular number, and a decrease in the trabecular separation (FIG. 47D). H&E-stained histological sections also exhibited a notable increase in the tumor-invaded area in Lrp5-deleted mice and a pronounced decrease by the administration of Lrp5-CM (FIG. 47E).

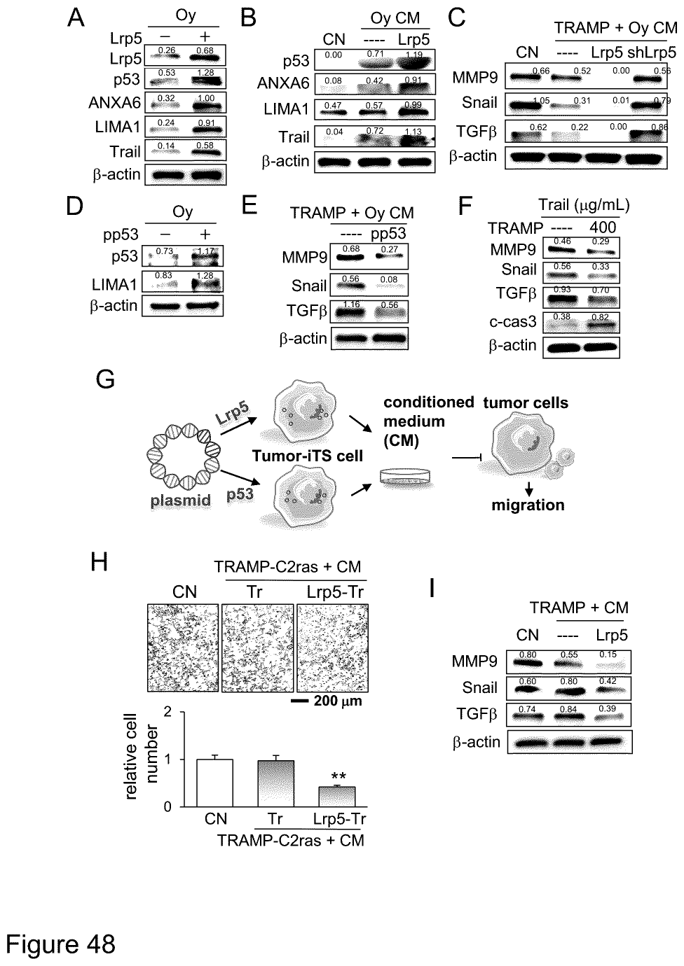

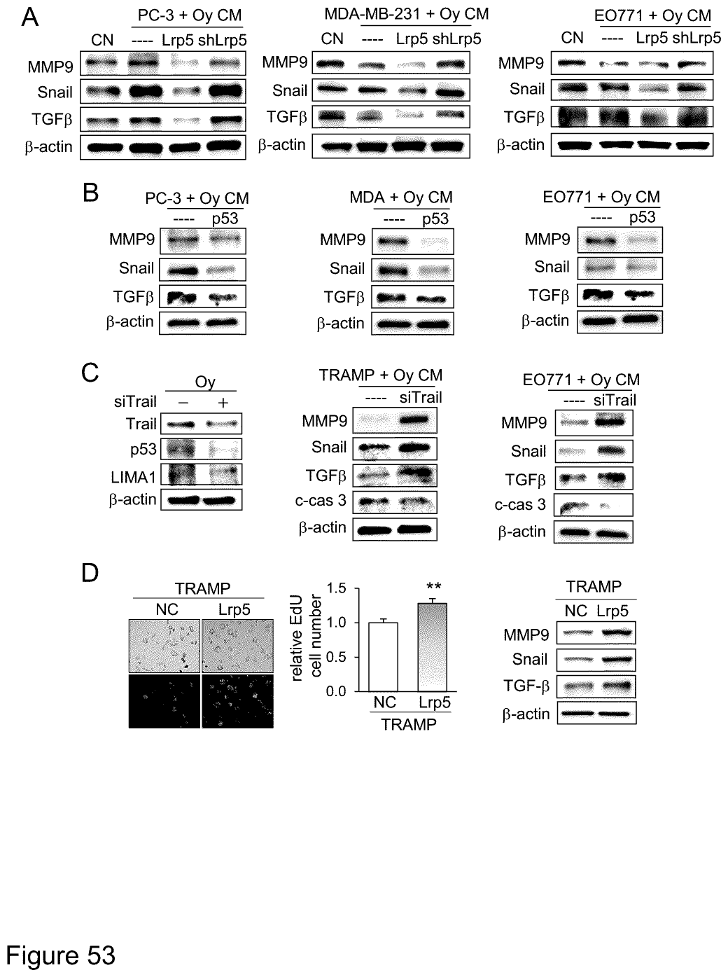

Elevation of TRAIL and p53 in Lrp5-CM. In vitro and in vivo results so far support the tumor-suppressing capability of Lrp5-CM. Western blot analysis revealed that the overexpression of Lrp5 in osteocytes (Lrp5-Oy) elevated the levels of potential tumor suppressors including, p53, ANXA6, and LIMA1, as well as TRAIL, an apoptosis-inducing factor, in osteocytes as well as in Lrp5-CM (FIGS. 48A and 48B). Lrp5-CM downregulated tumor-promoting genes, including MMP9, Snail, and TGFβ, in TRAMP tumor cells, while this downregulation was suppressed by Lrp5-silenced Oy CM (FIG. 48C). The same trend was observed with breast cancer cell lines (FIG. 53A).

To evaluate the role of p53 and TRAIL in Lrp5-CM, the levels of p53 and TRAIL were altered by p53 plasmids, TRAIL recombinant proteins, and TRAIL siRNAs. The result revealed that the elevation of p53 in Oy CM downregulated MMP9, Snail, and TGFβ in TRAMP, PC-3, MDA-MB-231, and EO771 tumor cells (FIG. 48E, FIG. 53B). The administration of TRAIL recombinant proteins downregulated MMP9, Snail, and TGFβ, with an increase in cleaved caspase 3, an apoptosis marker in TRAMP cells (FIG. 48F). Consistently, the silencing of TRAIL by siRNA in osteocytes suppressed the downregulation of MMP9, Snail, and TGFβ in TRAMP and EO771 tumor cells (FIG. 53C).

Generation of iTS cells from TRAMP prostate tumor cells. To further evaluate the effect of Lrp5 overexpression on tumor-suppressing capability, Lrp5 was overexpressed in TRAMP cells and applied Lrp5-TRAMP CM to TRAMP cells. The result revealed that the overexpression of Lrp5 converted TRAMP cells into iTS cells (FIG. 48G). TRAMP cells, treated with Lrp5-TRAMP CM, reduced TRANSWELL® invasion (FIG. 48H). Lrp5-TRAMP CM also downregulated MMP9, Snail, and TGFβ in TRAMP cells (FIG. 48I). On the contrary, Lrp5-TRAMP cells promoted EdU-based proliferation and elevated MMP9, Snail, and TGFβ (FIG. 53D). Collectively, the results support the notion that the overexpression of Lrp5 in tumor cells promoted their progression but their CM acted as an inhibitory agent to Lrp5-non-overexpressing tumor cells.

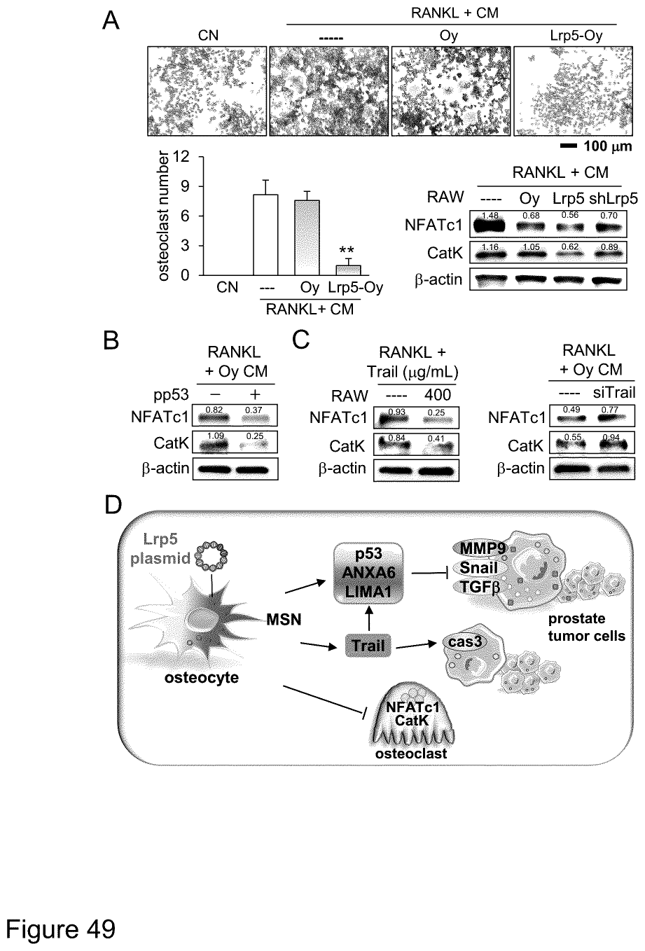

Inhibition of osteoclast development by Lrp5-CM. The results obtained support the tumor-suppressing and bone-protective capability of Lrp5-CM. Since osteoclasts are bone-resorbing cells, whether Lrp5 overexpression in osteocytes might affect the development of osteoclasts was examined. The result showed that the RANKL-driven development of RAW264.7 pre-osteoclasts was inhibited by Lrp5-CM, with the downregulation of NFATc1 and cathepsin K (FIG. 49A). Furthermore, the overexpression of p53 in Oy CM also reduced the level of NFATc1 and cathepsin K (FIG. 49B). The administration of recombinant TRAIL protein decreased their expression, while their reduction was suppressed by the silencing TRAIL in Oy CM (FIG. 49C).

### REFERENCES

Each of the following documents is hereby expressly incorporated by reference herein in its entirety.
Amarante-Mendes G P, Griffith T S. Therapeutic applications of TRAIL receptor agonists in cancer and beyond. Pharmacol Ther. 2015; 155: 117131. doi:10.1016/j.pharmthera.2015.09.001.
Anastas J N, Moon R T. WNT signalling pathways as therapeutic targets in cancer. Nat Rev Cancer. 13:11-26 (2013).
Berebichez-Fridman R, Montero-Olvera P R. Sources and Clinical Applications of Mesenchymal Stem Cells: State-of-the-art review. Sultan Qaboos Univ Med J 2018; 18(3):e264-e277. doi:10.18295/squmj.2018.18.03.002.
Berman A T, Thukral A D, Hwang W T, Solin L J, Vapiwala N. Incidence and patterns of distant metastases for patients with early-stage breast cancer after breast conservation treatment. Clin Breast Cancer. 2013; 13: 88-94.
Bonar S L, Brydges S D, Mueller J L, McGeough M D, Pena C, Chen D et al. Constitutively activated NLRP3 inflammasome causes inflammation and abnormal skeletal development in mice. 2012; PLoS One. 7(4): e35979.
Brinker A E, Vivian C J, Beadnell T C, Koestler D C, Teoh S T, Lunt S Y et al. Mitochondrial Haplotype of the Host Stromal Microenvironment Alters Metastasis in a Non-cell Autonomous Manner. Cancer Res 2020; 80: 1118-1129.
Buijs J T, et al. TGF-beta in the Bone Microenvironment: Role in Breast Cancer Metastases. Cancer Microenviron. 4:261-281 (2011).
Bullock W A, Pavalko F M, Robling A G. Osteocytes and mechanical loading: The Wnt connection. Orthod Craniofac Res 2019; 22 Suppl 1: 175-179.
Buenrostro D, Mulcrone P L, Owens P, Sterling J A. The Bone Microenvironment: a Fertile Soil for Tumor Growth. Curr Osteoporos Rep 2016; 14: 151-158.
Carpenter K A, Ross R D. Sclerostin Antibody Treatment Increases Bone Mass and Normalizes Circulating Phosphate Levels in Growing Hyp Mice. J Bone Miner Res 2020; 35: 596-607.
Chen A, Wang L, Li B Y, Sherman J, Ryu J E, Hamamura K, Liu Y, Nakshatri H, Yokota H. Reduction in Migratory Phenotype in a Metastasized Breast Cancer Cell Line via Downregulation of S100A4 and GRM3. Sci Rep. 2017; 7: 3459. doi:10.1038/s41598-017-03811-9.
Chen A, Wang L, Liu S, Wang Y, Liu Y, Wang M et al. Attraction and Compaction of Migratory Breast Cancer Cells by Bone Matrix Proteins through Tumor-Osteocyte Interactions. Sci Rep. 2018; 8 (1): 5420.
Connelly, K. E., V. Hedrick, T. J. Paschoal Sobreira, E. C. Dykhuizen, and U. K. Aryal. Analysis of Human Nuclear Protein Complexes by Quantitative Mass Spectrometry Profiling. Proteomics 2018; 18(11): e1700427.
Cox, J., and M. Mann. MaxQuant enables high peptide identification rates, individualized p.p.b.-range mass accuracies and proteome-wide protein quantification. Nat Biotechnol 2008; 26(12): 1367-1372.
Cui Y X, Evans B A, Jiang W G. New Roles of Osteocytes in Proliferation, Migration and Invasion of Breast and Prostate Cancer Cells. Anticancer Res 2016; 36: 1193-1201.
Custòdio-Santos T, Videira M, Brito M A. Brain metastasization of breast cancer. BBA Reviews on Cancer. 2017; 1868: 132-147.
Dabovic B, Chen Y, Colarossi C, Zambuto L, Obata H, Rifkin D B. Bone defects in latent TGF-beta binding protein (Ltbp)-3 null mice; a role for Ltbp in TGF-beta presentation. J Endocrinol. 2002; 175 (1): 129-41.
Dallas S L, Prideaux M, Bonewald L F. The osteocyte: an endocrine cell . . . and more. Endocr Rev. 34:658-690 (2013).
Daneman R, Prat A. The Blood-Brain Barrier. Cold Spring Harb Perspect Biol. 2015; 7 (1): a020412.7.
DeSantis C E, Fedewa S A, Goding Sauer A, Kramer J L, Smith R A, Jemal A. Breast cancer statistics, 2015: Convergence of incidence rates between black and white women. CA Cancer J Clin 2016; 66: 1-42. doi:10.3322/caac.21320.
da Silva Araujo A, Moraes A M, Lourenco M C S, Pessoa C O, da Silva E T, de Souza M V N. Synthesis and Antibacterial Activity of Mefloquine-Based Analogs Against Sensitive and Resistant Mycobacterium tuberculosis Strains. Curr Top Med Chem 2019; 19: 683-689.
De Craene B, Berx G. Snail in the frame of malignant tumor recurrence. Breast Cancer Res. 8:105 (2006).
Delgado-Calle, et al. Genetic deletion of Sost or pharmacological inhibition of sclerostin prevent multiple myeloma-induced bone disease without affecting tumor growth. Leukemia. 31(12):2686-2694 (2017).
Di J, et al. Identification of WISP1 as a novel oncogene in glioblastoma. International Journal of Oncology. 51(4):1261-1270 (2017).
Drake M T, Clarke B L, Oursler M J, Khosla S. Cathepsin K Inhibitors for Osteoporosis: Biology, Potential Clinical Utility, and Lessons Learned. Endocr Rev 2017; 38: 325-350.
Duchartre Y, Kim Y M, Kahn M. The Wnt signaling pathway in cancer. Crit Rev Oncol Hematol. 2016; 99: 141-149.
Ella B, et al. Ronit Shtrichman Cell-based therapy approaches: the hope for incurable diseases. Regen Med. 9(5):649-72 (2014).
Erdogan B, Cicin I. Medical treatment of breast cancer bone metastasis: from bisphosphonates to targeted drugs. Asian Pac J Cancer Prev 2014; 15(4):1503-10. doi: 10.7314/apj cp 0.2014.15.4.1503.
Ewens A, Mihich E, Ehrke M J. Distant metastasis from subcutaneously grown EO771 medullary breast adenocarcinoma. Anticancer Res. 2005; 25 (6B): 3905-3915.
Fabbi M, Carbotti G, Ferrini S. Dual Roles of IL-27 in Cancer Biology and Immunotherapy. Mediators Inflamm2017; 2017:3958069. doi:10.1155/2017/3958069.
Fan Y, Jalali A, Chen A, Zhao X, Liu S, Teli M et al. Skeletal loading regulates breast cancer-associated osteolysis in a loading intensity-dependent fashion. Bone Res 2020; 8: 9.
Feng C, Zhang H L, Zeng A, Bai M, Wang X J. Tumor-Suppressive MicroRNA-216b Binds to TPX2, Activating the p53 Signaling in Human Cutaneous Squamous Cell Carcinoma. Mol Ther Nucleic Acids 2020; 20: 186-195.
Ferrari N, McDonald L, Morris J S, Cameron E R, Blyth K. Runx2 in mammary gland development and breast cancer. Int J Biol Macromol. 2013; 99:608-614.
Fidler I J. The Biology of Brain Metastasis: Challenges for Therapy. Cancer J. 2015; 21 (4): 284-293.
Fizazi K, Carducci M, Smith M, Damiao R, Brown J, Karsh L et al. Denosumab versus zoledronic acid for treatment of bone metastases in men with castration-resistant prostate cancer: a randomised, double-blind study. Lancet 2011; 377: 813-822.
Florencio-Silva R, Sasso G R, Sasso-Cerri E, SimOes M J, Cerri P S. Biology of bone tissue: structure, function, and factors that influence bone cells. Biomed Res Int 2015; 2015:421746. doi:10.1155/2015/421746.
Fornetti J, Welm A L, Stewart S A. Understanding the Bone in Cancer Metastasis. J Bone Miner Res 2018; 33: 2099-2113.
Ganguly S S, Li X, Miranti C K. The host microenvironment influences prostate cancer invasion, systemic spread, bone colonization, and osteoblastic metastasis. Front Oncol 2014; 4: 364.
Gao H, Zhai M, Wang P, Zhang X, Cai J, Chen X et al. Low-level mechanical vibration enhances osteoblastogenesis via a canonical Wnt signaling-associated mechanism. Mol Med Rep. 2013; 16 (1): 317-324.
Goldring S R. The osteocyte: key player in regulating bone turnover. RMD Open. 1:e000049 (2015).
Grozescu T, Popa F. Prostate cancer between prognosis and adequate/proper therapy. J Med Life 2017; 10: 5-12.
Gunawardena T N A, Rahman M T, Abdullah B J J, Abu Kasim N H. Conditioned media derived from mesenchymal stem cell cultures: The next generation for regenerative medicine. J Tissue Eng Regen Med. 2019 April; 13(4):569-586. doi: 10.1002/term.2806.
Guo F, Long L, Wang J, Wang Y, Liu Y, Wang L et al. Insights on CXC chemokine receptor 2 in breast cancer: An emerging target for oncotherapy. Oncol Lett. 18 (6): 5699-5708 (2019).
Hamamura K, Minami K, Tanjung N, Wan Q, Koizumi M, Matsuura N et al. Attenuation of malignant phenotypes of breast cancer cells through eIF2α-mediated downregulation of Racl signaling. Int J Oncol. 2014; 44 (6): 1980-1988.
Hamamura K., et al. Knee loading reduces MMP13 activity in the mouse cartilage. BMC Musculoskelet Disord. 14:312 (2013).
Han L, Li Z, Jiang Y, Jiang Z, Tang L. SNHG29 regulates miR-223-3p/CTNND1 axis to promote glioblastoma progression via Wnt/beta-catenin signaling pathway. Cancer Cell Int 2019; 19: 345.
Hao Y, Baker D, Ten Dijke P. TGF-β-Mediated Epithelial-Mesenchymal Transition and Cancer Metastasis. Int J Mol Sci. 2019; 20 (11):2767.
Hardaway A L, Herroon M K, Raj agurubandara E, Podgorski I. Marrow Adipocyte-derived CXCL1, and CXCL2 Contribute to Bone Osteolysis in Metastatic Prostate Cancer. Clin Exp Metastasis. 2015; 32(4): 353-368. doi:10.1007/s10585-015-9714-5.
Hass R, Otte A. Mesenchymal stem cells as all-round supporters in a normal and neoplastic microenvironment. Cell Commun Signal. 2012; 10(1):26. doi:10.1186/1478-811X-10-26.
He N, Kong Y, Lei X, Liu Y, Wang J, Xu C, Wang Y, Du L, Ji K, Wang Q, et al. MSCs inhibit tumor progression and enhance radiosensitivity of breast cancer cells by downregulating Stat3 signaling pathway. Cell Death Dis 2018; 9(10):1026. doi:10.1038/s41419-018-0949-3.
Holen I, Lefley D V, Francis S E, Rennicks S, Bradbury S, Coleman R E et al. IL-1 drives breast cancer growth and bone metastasis in vivo. Oncotarget. 2016; 7 (46): 75571-75584.
Homare E, et al. Regulation of Sclerostin Expression in Multiple Myeloma by Dkk-1: A Potential Therapeutic Strategy for Myeloma Bone Disease. J Bone Miner Res. 31(6):1225-34 (2016).
Howe, E. N., M. D. Burnette, M. E. Justice, P. M. Schnepp, V. Hedrick, J. W. Clancy, I. H. Guldner, A. T. Lamere, J. Li, et al. Rab 1 lb-mediated integrin recycling promotes brain metastatic adaptation and outgrowth. Nat Commun 2020; 11(1): 3017.
Hsu J L, Pan S L, Ho Y F, Hwang T L, Kung F L, Guh J H. Costunolide induces apoptosis through nuclear calcium2+ overload and DNA damage response in human prostate cancer. J Ural 2011; 185: 1967-1974.
Hsu Y L, et al. Breast tumor-associated osteoblast-derived CXCL5 increases cancer progression by ERK/MSK1/Elk-1/snail signaling pathway. Oncogene. 32:4436-4447 (2013).
Hurst L D, Atlan A, Bengtsson B O. Genetic conflicts. Q Rev Biol. 1996; 71(3):317-64. doi:10.1086/419442.
Ikuno Uehara, Nobuyuki Tanaka. Role of p53 in the Regulation of the Inflammatory Tumor Microenvironment and Tumor Suppression. Cancers (Basel). 10(7):219 (2018).
Janane A, Hajji F, Ismail T, Jawad C, Crepin-Elondo J, Ghadouane M et al. [Bone mineral density change: comparison between prostate cancer patients with or without metastases and healthy men (a North African ethnic group)]. Actas Urol Esp 2011; 35: 414-419.
Jeanne A P. Prospect of Stem Cell Conditioned Medium in Regenerative Medicine. Biomed Res Int 2014; 2014:965849.
Jeet V, Ow K, Doherty E, Curley B, Russell P J, Khatri A. Broadening of transgenic adenocarcinoma of the mouse prostate (TRAMP) model to represent late-stage androgen depletion independent cancer. Prostate 2008; 68: 548-562.
Johnstone C N, Pattison A D, Harrison P F, Powell D R, Lock P, Ernst M et al. FGF13 promotes metastasis of triple-negative breast cancer. Int J Cancer 2020.
Joiner D M, Ke J, Zhong Z, Xu X E, Williams B O. LRP5 and LRP6 in development and disease. Trends Endocrinol Metab. 2013; 24 (1): 31-39.
Jones S, Zhang X, Parsons D W, Lin J C, Leary R J, Angenendt P, Mankoo P, Carter H, Kamiyama H, Jimeno A, et al. Core signaling pathways in human pancreatic cancers revealed by global genomic analyses. Science. 2008; 321(5897):1801-6. doi:10.1126/science.1164368.
Joseph A., et al. Mesenchymal stem cell-conditioned media: A novel alternative of stem cell therapy for quality wound healing. J Cell Physiol. 235(7-8):5555-5569 (2020).
Kakonen S M, Mundy G R. Mechanisms of osteolytic bone metastases in breast carcinoma. Cancer 2003; 97: 834-839.
Kang K S, Hong J M, Horan D J, Lim K E, Bullock W A, Bruzzaniti A et al. Induction of Lrp5 HBM-causing mutations in Cathepsin-K expressing cells alters bone metabolism. Bone 2019; 120: 166-175.
Kang K S, Hong J M, Robling A G. Postnatal beta-catenin deletion from Dmp1-expressing osteocytes/osteoblasts reduces structural adaptation to loading, but not periosteal load-induced bone formation. Bone 2016; 88: 138-145.
Katoh M, Katoh M. WNT Signaling Pathway and Stem Cell Signaling Network. Clin Cancer Res. 2007; 13 (14): 4042-4045.
Kenkre J S, Bassett J H. The Bone Remodeling Cycle. Ann Clin Biochem. 2018; 55 (3): 308-327.
Klein-Nulend J., et al. Mechanosensation and transduction in osteocytes. Bone. 54:182-190 (2013).
Kotrbova A, Ovesna P, Gybel T, Radaszkiewicz T, Bednarikova M, Hausnerova J et al. WNT signaling inducing activity in ascites predicts poor outcome in ovarian cancer. Theranostics 2020; 10: 537-552.
Kuhn E, Morbini P, Cancellieri A, Damiani S, Cavazza A, Comin C E. Adenocarcinoma classification: patterns and prognosis. Pathologica 2018; 110: 5-11.
Lau K H, Baylink D J, Zhou X D, Rodriguez D, Bonewald L F, Li Z et al. Osteocyte-derived insulin-like growth factor I is essential for determining bone mechanosensitivity. Am J Physiol Endocrinol Metab 2013; 305: E271-281.
Layman A A K, Joshi S, Shah S. Metastatic prostate cancer presenting as tumour-induced osteomalacia. BMJ Case Rep 2019; 12.
Lee S I, Roney M S I, Park J H, Baek J Y, Park J, Kim S K et al. Dopamine Receptor Antagonists Induce Differentiation of PC-3 Human Prostate Cancer Cell-Derived Cancer Stem Cell-Like Cells. Prostate 2019; 79(7):720-731.
Lelekakis M, Moseley J M, Martin T J, Hards D, Williams E, Ho P, Lowen D, Javni J, Miller F R, Slavin J, et al. A novel orthotopic model of breast cancer metastasis to bone. Clinical& Experimental metastasis. March 1999, Volume 17, Issue 2, pp 163-17.
Lewis Phillips G D, Nishimura M C, Lacap J A, Kharbanda S, Mai E, Tien J et al. Trastuzumab Uptake and Its Relation to Efficacy in an Animal Model of HER2-positive Breast Cancer Brain Metastasis. Breast Cancer Res Treat. 2017; 164 (3): 581-591.
Li F, Chen A, Reeser A, Wang Y, Fan Y, Liu S, Zhao X, Prakash R, Kota D, Li B Y, et al. Vinculin force sensor detects tumor-osteocyte interactions. Scientific Reports 9,5615 (2018).
Li X, Yang Q, Yu H, Wu L, Zhao Y, Zhang C, Yue X, Liu Z, Wu H, Haffty B G, et al. LIF promotes tumorigenesis and metastasis of breast cancer through the AKT-mTOR pathway. Oncotarget. 2014; 5(3): 788-801. doi:10.18632/oncotarget.1772.
Liu S, Fan Y, Chen A, Jalali A, Minami K, Ogawa K et al. Osteocyte-Driven Downregulation of Snail Restrains Effects of Drd2 Inhibitors on Mammary Tumor Cells. Cancer Res 2018; 78: 3865-3876.
Liu S, Liu Y, Minami K, Chen A, Wan Q, Yin Y, Gan L, Xu A, Matsuura N, Koizumi M, et al. Inhibiting checkpoint kinase 1 protects bone from bone resorption by mammary tumor in a mouse model. Oncotarget. 2018; 9(10):9364-9378. doi:10.18632/oncotarget.24286.
Liu Y, Xin T, Jiang Q Y, Huang D Y, Shen W X, Li L et al. CD147, MMP9 expression and clinical significance of basal-like breast cancer. Med Oncol. 2013; 30 (1):366.
Lorger M, Felding-Habermann B. Capturing changes in the brain microenvironment during initial steps of breast cancer brain metastasis. Am J Pathol. 2010; 176: 2958-2971.
Mabilleau G, Mieczkowska A, Libouban H, Simon Y, Audran M, Chappard D. Comparison between quantitative X-ray imaging, dual-energy X-ray absorptiometry and microCT in the assessment of bone mineral density in disuse-induced bone loss. J Musculoskelet Neuronal Interact. 2015 March; 15(1):42-52. PMID: 25730651; PMCID: PMC5123607.
MacDonald BT, Tamai K, He X. Wnt/beta-catenin signaling: components, mechanisms, and diseases. Dev Cell. 17:9-26 (2009).
Maeda K, Kobayashi Y, Koide M, Uehara S, Okamoto M, Ishihara A et al. The Regulation of Bone Metabolism and Disorders by Wnt Signaling. Int J Mot Sci 2019; 20.
Marando C M, Park C Y, Liao J A, Lee J K, Chuck R S. Revisiting the Cornea and Trabecular Meshwork Junction With 2-Photon Excitation Fluorescence Microscopy. Cornea 2017; 36: 704-711.
Martin M., et al. Mechanobiological osteocyte feedback drives mechanostat regulation of bone in a multiscale computational model. Biomech Model Mechanobiol. 18:1475-1496 (2019).
Mello S S, Valente L J, Raj N, Seoane J A, Flowers B M, McClendon J. A p53 super-tumor suppressor reveals a tumor-suppressive p53-Ptpn14-Yap axis in pancreatic cancer. Cancer Cell. 2017; 32(4): 460-473. doi:10.1016/j.ccell.2017.09.007.
Melzer C, Yang Y, Hass R. Interaction of MSC with tumor cells. Cell Commun Signal. 2016; 14(1):20. doi:10.1186/s12964-016-0143-0.
Mikyšková R, Štěpanek I, Indrová M, Bieblová J, Šimová J, Truxová I, Moserová I, Fučiková J, Bartn̊ňková J, Špíšek R, et al. Dendritic cells pulsed with tumor cells killed by high hydrostatic pressure induce strong immune responses and display therapeutic effects both in murine TC-1 and TRAMP-C2 tumors when combined with docetaxel chemotherapy. Int J Oncol. 2016; 48(3): 953-964. doi:10.3892/ijo.2015.3314.
Miller K D, Weathers T, Haney L G, Timmerman R, Dickler M, Shen J et al. Occult central nervous system involvement in patients with metastatic breast cancer: Prevalence, predictive factors and impact on overall survival. Ann Oncol. 2003; 14 (7): 1072-1077.
Minami K, Liu S, Liu Y, Chen A, Wan Q, Na S, Li B Y, Matsuura N, Koizumi M, Yin Y, et al. Inhibitory Effects of Dopamine Receptor D1 Agonist on Mammary Tumor and Bone Metastasis. Sci Rep. 2017; 7: 45686. doi:10.1038/srep45686.
Nie Z, Du M Q, McAllister-Lucas L M, Lucas P C, Bailey N G, Hogaboam C M et al. Conversion of the LIMA1 tumor suppressor into an oncogenic LMO-like protein by API2-MALT1 in MALT lymphoma. Nat Commun. 2015; 6: 5908.
Nusse R, Clevers H. Wnt/f3-Catenin Signaling, Disease, and Emerging Therapeutic Modalities. Cell. 2017; 169 (6): 985-999.
Olivero C E, Martinez-Terroba E, Zimmer J, Liao C, Tesfaye E, Hooshdaran N et al. p53 Activates the Long Noncoding RNA Pvtlb to Inhibit Myc and Suppress Tumorigenesis. Mol Cell 2020; 77: 761-774 e768.
Opoku-Temeng, C., K. I. Onyedibe, U. K. Aryal, and H. O. Sintim. Proteomic analysis of bacterial response to a 4-hydroxybenzylidene indolinone compound, which re-sensitizes bacteria to traditional antibiotics. J Proteomics 2019; 202: 103368.
Pai S G, Carneiro B A, Mota J M, Costa R, Leite C A, Barroso-Sousa R, Kaplan J B, Chae Y K, Giles F J. Wnt/beta-catenin pathway: modulating anticancer immune response. J Hematol Oncol. 2017 May 5; 10(1):101. doi: 10.1186/s13045-017-0471-6. disaggregation. Nat Rev Mol Cell Biol. 2013 October; 14(10):630-42. doi: 10.1038/nrm3658.
Paranhos-Neto F P, Vieira Neto L, Madeira M, Moraes A B, Mendonca L M C, Lima I C B et al. Vitamin D deficiency is associated with cortical bone loss and fractures in the elderly. Eur J Endocrinol 2019; 181: 509-517.
Parker T M, et al. Cell competition and tumor heterogeneity. Semin Cancer Biol. 63:1-10. (2020).
Paster E V, Villines K A, Hickman D L. Endpoints for mouse abdominal tumor models: refinement of current criteria. Comp Med. 2009; 59 (3): 234-241.
Pham P. V., et al. Targeting breast cancer stem cells by dendritic cell vaccination in humanized mice with breast tumor: preliminary results. Onco Targets Ther. 9:4441-4451 (2016).
Pietsch E C, Sykes S M, McMahon S B, Murphy M E. The p53 family and programmed cell death. Oncogene. 2008; 27 (50): 6507-21.
Prasad M., et al. Dual TGFbeta/BMP Pathway Inhibition Enables Expansion and Characterization of Multiple Epithelial Cell Types of the Normal and Cancerous Breast. Mol Cancer Res. 17:1556-1570 (2019).
Quayle L, Ottewell P D, Holen I. Bone Metastasis: Molecular Mechanisms Implicated in Tumour Cell Dormancy in Breast and Prostate Cancer. Curr Cancer Drug Targets 2015; 15: 469-480.
Rawla P. Epidemiology of Prostate Cancer. World J Oncol 2019; 10: 63-89.
Refaat A, Abd-Rabou A, Reda A. TRAIL combinations: The new ‘trail’ for cancer therapy (Review). Oncol Lett. 2014; 7 (5): 1327-1332.
Ribot E. J., et al. 3D anatomical and perfusion MRI for longitudinal evaluation of biomaterials for bone regeneration of femoral bone defect in rats. Sci Rep. 7:6100 (2017).
Roodman G D. Mechanisms of bone metastasis. N Engl J Med. 2004; 350(16):1655-64.
Roslan Z, Muhamad M, Selvaratnam L, Ab-Rahim S. The roles of low-density lipoprotein receptor-related proteins 5, 6, and 8 in Cancer: A Review. J Oncol 2019; 2019:4536302. doi:10.1155/2019/4536302.
Rossi L, Longhitano C, Kola F, Del Grande M. State of art and advances on the treatment of bone metastases from breast cancer: a concise review. Chin Clin Oncol 2020.01.07. doi:10.21037/cco.2020.01.07.
Satterlee A B, Dunn D E, Lo D C, Khagi S, Hingtgen S. Tumoricidal stem cell therapy enables killing in novel hybrid models of heterogeneous glioblastoma. Neuro Oncol 2019; 21: 1552-1564.
Sawakami K., et al. The Wnt co-receptor LRP5 is essential for skeletal mechanotransduction but not for the anabolic bone response to parathyroid hormone treatment. J Biol Chem. 281:23698-23711 (2006).
Schcolnik-Cabrera A, Oldak B, Juarez M, Cruz-Rivera M, Flisser A, Mendlovic F. Calreticulin in phagocytosis and cancer: opposite roles in immune response outcomes. Apoptosis. 2019 April; 24(3-4):245-255. doi: 10.1007/s10495-019-01532-0.
Sekita A, Matsugaki A, Nakano T. Disruption of collagen/apatite alignment impairs bone mechanical function in osteoblastic metastasis induced by prostate cancer. Bone. 97:83-93 (2017).
Sharif Y, Jumah F, Coplan L, Krosser A, Sharif K, Tubbs R S. Blood-brain barrier: a review of its anatomy and physiology in health and disease. Clin Anat. 2018; 31: 812-823.
Siegel R, Naishadham D, Jemal A. Cancer statistics, 2013. CA Cancer J Clin. 2013; 63:11-30.
Sorlie, T. The Impact of Gene Expression Patterns in Breast Cancer. Clin Chem. 2016; 62 (8): 1150-1151.
Spatz J M, Wein M N, Gooi J H, Qu Y, Garr J L, Liu S et al. The Wnt Inhibitor Sclerostin Is Up-regulated by Mechanical Unloading in Osteocytes in Vitro. J Biol Chem 2015; 290: 16744-16758.
Sun J, Liu Y, Chen Y, Zhao W, Zhai Q, Rathod S et al. Doxorubicin delivered by a redox-responsive dasatinib-containing polymeric prodrug carrier for combination therapy. J Control Release 2017; 258: 43-55.
Taciak B, Pruszynska I, Kiraga L, Bialasek M, Krol M. Wnt signaling pathway in development and cancer. J Physiol Pharmacol 2018; 69.
Tai S, Sun Y, Squires J M, Zhang H, Oh W K, Liang C Z, Huang J. PC3 is a cell line characteristic of prostatic small cell carcinoma. Prostate 2011; 71(15): 1668-79. doi:10.1002/pros.21383.
Taipale M, Jarosz D F, Lindquist S. HSP90 at the hub of protein homeostasis: emerging mechanistic insights. Nat Rev Mol Cell Biol. 2010 July; 11(7):515-28. doi: 10.1038/nrm2918.
Tawara K, Scott H, Emathinger J, Wolf C, LaJoie D, Hedeen D et al. High expression of OSM and IL-6 are associated with decreased breast cancer survival: synergistic induction of IL-6 secretion by OSM and IL-1β. Oncotarget. 2019; 10 (21): 2068-2085.
Torrel L A, Siegel R L, Ward E M, Jemal A. Global Cancer Incidence and Mortality Rates and Trends—An Update. Cancer Epidemiol Biomarkers Prev. 2016; 25 (1): 16-27.
Uccelli A, Moretta L, Pistoia V. Mesenchymal stem cells in health and disease. Nat Rev Immunol 2008; 8(9):726-36. doi: 10.1038/nri2395.
Ulsperger E., et al. Effects of 1alpha,25-dihydroxy-vitamin D3 pretreatment and MAP kinase inhibitor PD 98059 on response of osteoblasts to prostate-derived osteoblastic factors. Oncol Rep. 10:1529-1534 (2003).
Valencia K, Ormazabal C, Zandueta C, Luis-Ravelo D, Antón I, Pajares M J et al. Inhibition of collagen receptor discoidin domain receptor-1 (DDR1) reduces cell survival, homing, and colonization in lung cancer bone metastasis. Clin Cancer Res 2012; 18(4):969-980.
Vandercappellen J, Van Damme J, Struyf S. The role of CXC chemokines and their receptors in cancer. Cancer Lett. 2008; 267 (2): 226-244.
Verkhratsky A, Sofroniew M V, Messing A, deLanerolle NC, Rempe D, Rodriguez J J et al. Neurological diseases as primary gliopathies: a reassessment of neurocentrism. ASN Neuro. 2012; 4 (3): e00082.
Wang L, Wang Y, Chen A, Jalali A, Liu S, Guo Y, Na S, Nakshatri H, Li B Y, Yokota H. Effects of a checkpoint kinase inhibitor, AZD7762, on tumor suppression and bone remodeling. Int J Oncol 2018; 53(3):1001-1012. doi:10.3892/ijo.2018.4481.
Wang L, Wang F S. Clinical immunology and immunotherapy for hepatocellular carcinoma: current progress and challenges. Hepatol Int. 13:521-533 (2019).
Wang M, Feng L, Li P, Han N, Gao Y, Xiao T. Hsp90AB1 Protein is Overexpressed in Non-small Cell Lung Cancer Tissues and Associated with Poor Prognosis in Lung Adenocarcinoma Patients. Zhongguo Fei Ai Za Zhi. 2016 February; 19(2):64-9. doi: 10.3779/j.i ssn.1009-3419.2016.02.02.
Wang N., et al. CXCL1 derived from tumorWang H, Deng G, Ai M, Xu Z, Mou T, Yu J, Liu H, Wang S, Li G. Hsp90ab1 stabilizes LRP5 to promote epithelial-mesenchymal transition via activating of AKT and Wnt/β-catenin signaling pathways in gastric cancer progression. Oncogene. 2019 February; 38(9):1489-1507. doi: 10.1038/s41388-018-0532-5.
Wang Q, Mo J, Zhao C, Huang K, Feng M, He W, Wang J, Chen S, Xie Z, Ma J, et al. Raddeanin A suppresses breast cancer-associated osteolysis through inhibiting osteoclasts and breast cancer cells. Cell Death Dis 2018; 9(3):376. doi:10.1038/s41419-018-0417-0.
Wang W, Yang X, Dai J, Lu Y, Zhang J, Keller E T. Prostate cancer promotes a vicious cycle of bone metastasis progression through inducing osteocytes to secrete GDF15 that stimulates prostate cancer growth and invasion. Oncogene 2019; 38: 4540-4559.
Wang Y, Shi J, Chai K, Ying X, Zhou B P. The role of snail in EMT and tumorigenesis. Curr Cancer Drug Targets. 2013; 13:963-72.
Weigelt B., et al. Breast cancer metastasis: markers and models. Nat Rev Cancer. 5:591-602 (2005).
Weivoda M M, Ruan M, Hachfeld C M, Pederson L, Howe A, Davey R A et al. Wnt Signaling Inhibits Osteoclast Differentiation by Activating Canonical and Noncanonical cAMP/PKA Pathways. J Bone Miner Res 2016; 31: 65-75.
Wilson C, Bell R, Hinsley S, Marshall H, Brown J, Cameron D et al. Adjuvant zoledronic acid reduces fractures in breast cancer patients; an AZURE (BIG 01/04) study. Eur J Cancer 2018; 94: 70-78.

1. A pharmaceutical composition comprising
a cell-free conditioned medium (CM) or extract or concentrate thereof obtained from a mammalian cell culture medium comprising a cultured substantially homogenous non-cancerous mammalian cell population;
wherein at least a portion of the non-cancerous mammalian cell population is contacted by at least one small molecule cell growth signaling pathway activator before being cultured in the cell culture medium.

2. (canceled)

3. The pharmaceutical composition of claim 1, wherein the at least a portion of the non-cancerous mammalian cell population is contacted by at least two small molecule cell growth signaling pathway activators before being cultured in the cell culture medium.

4. (canceled)

5. The pharmaceutical composition of claim 1, wherein the conditioned medium further comprises a non-cancerous mammalian cell-secreted protein selected from the group consisting of Hsp90ab1, Calr, Ppia, Ppib, Flna, H4, Vim, and Ubc.

6. (canceled)

7. (canceled)

8. The pharmaceutical composition of claim 1, wherein the small molecule cell growth signaling pathway activator is a small molecule Wnt signaling pathway activator.

9. The pharmaceutical composition of claim 8, wherein the small molecule Wnt signaling pathway activator is BML-284, or a pharmaceutically acceptable salt thereof.

10. The pharmaceutical composition of claim 1, wherein the non-cancerous mammalian cells are non-cancerous mammalian bone cells.

11. (canceled)

12. (canceled)

13. A kit comprising:
a pharmaceutical composition comprising
a cell-free conditioned medium (CM) or extract or concentrate thereof obtained from a mammalian cell culture medium comprising a cultured substantially homogenous non-cancerous mammalian cell population;
wherein at least a portion of the non-cancerous mammalian cell population is contacted by at least one small molecule cell growth signaling pathway activator before being cultured in the cell culture medium;
a container;
a label; and
instructions that provide methods for administering the composition.

14. (canceled)

15. A method to treat a cancer in a subject in need thereof, the method comprising:
administering to the subject in need thereof a therapeutically effective amount of a cell-free conditioned medium (CM) or extract or concentrate thereof obtained from a mammalian cell culture medium comprising a cultured substantially homogenous non-cancerous mammalian cell population;
wherein a portion of the non-cancerous mammalian cell population is contacted by a small molecule cell growth signaling pathway activator before being cultured in the cell culture medium.

16. (canceled)

17. The method of claim 15, wherein the treated cancer is a metastatic cancer and the cultured substantially homogenous non-cancerous mammalian cell population is derived from a same organ or tissue as the organ or site to be treated.

18. (canceled)

19. (canceled)

20. The method of claim 15, wherein the treated cancer is a metastatic bone cancer.

21. The method of claim 15, wherein the treated cancer is a primary cancer.

22. (canceled)

23. (canceled)

24. (canceled)

25. The method of claim 15, wherein the small molecule cell growth signaling pathway activator is selected from the group consisting of a small molecule Wnt signaling pathway activator, a small molecule PI3K signaling pathway activator, a small molecule FGF signaling pathway activator and a small molecule Notch signaling pathway activator.

26. The method of claim 15, wherein the small molecule cell growth signaling pathway activator is a small molecule Wnt signaling pathway activator.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. The method of claim 15, wherein the method results in at least one activity selected from the group consisting of upregulating a tumor-suppressing gene in a cancer cell, downregulating a tumor-promoting gene in a cancer cell, inhibiting cancer cell invasion, inhibiting cancer cell growth, and inhibiting cancer cell recurrence.

41. The method of claim 15, wherein the target cancer is a therapy-resistant cancer.

42. A process to produce a conditioned medium (CM), the process comprising:
contacting non-cancerous mammalian cells with a small molecule cell growth signaling pathway activator to generate pre-treated non-cancerous mammalian cells;
culturing the pre-treated non-cancerous mammalian cells in a mammalian cell culture medium for a period of time sufficient to condition the medium;
removing the pre-treated non-cancerous mammalian cells from the culture medium; and,
collecting the conditioned medium.

43. The process of claim 42, wherein the collected conditioned medium comprises a non-cancerous mammalian cell-secreted protein selected from the group consisting of Hsp90ab1, Calr, Ppia, Ppib, Flna, H4, Vim, and Ubc.

44. The process of claim 42, wherein the mammalian cell culture medium is serum-free.

45. The process of claim 42, wherein the pre-treated non-cancerous mammalian cells are cultured for a time period from about 1 hour to about 4 hours.

46. The process of claim 42, further comprising filtering the collected conditioned medium.

47. (canceled)

48. The process of claim 42, further comprising concentrating the collected conditioned medium.

49. (canceled)

50. (canceled)

51. (canceled)
